(19) United States
(12) Patent Application Publication
(10) Pub. No.: US 2017/0036969 A1
(43) Pub. Date: Feb. 9, 2017

(54) NITRIFICATION INHIBITORS

(71) Applicant: BASF SE, Ludwigshafen (DE)

(72) Inventors: Barbara NAVE, Ruppertsberg (DE); Joachim DICKHAUT, Heidelberg (DE); Mihiret Tekeste SISAY, Mannheim (DE); Alexander WISSEMEIER, Speyer (DE); Wolfram ZERULLA, St Martin (DE); Johan EBENHOECH, Mannheim (DE); Wolfgang WEIGELT, Dudenhofen (DE)

(21) Appl. No.: 15/304,129

(22) PCT Filed: Apr. 16, 2015
(86) PCT No.: PCT/EP2015/058316

§ 371 Date: Oct. 14, 2016

(30) Foreign Application Priority Data
Apr. 17, 2014 (EP) 14165216.4

(51) Int. Cl. C05G 3/08; C07C 255/54; C07C 333/08; C07C 217/84; C07D 249/14; C07D 513/04; C07C 43/215; C07C 271/58; C07D 241/24; C07C 233/25; C07D 241/18; C07C 251/48; C07D 257/04; C07D 249/12; C07C 275/34; C07C 205/37; C07D 233/86; C07D 401/12; C07D 213/73; C07C 255/65; C07C 243/22; C07C 69/92; C07C 243/28; C07C 251/76; C07C 49/255; C07C 47/575; C07D 277/68; C07C 43/23; C07D 253/075; C07C 335/18; C07D 307/68; C07D 495/04; C07C 311/37; C07D 239/34; C07D 209/34; C07D 295/205; C07D 231/56; C07D 277/54; C07D 211/16; C07D 275/04; C07D 487/04; C07D 239/545; C07D 401/04; C07D 239/42; C07D 275/03; C07D 261/20; C07D 311/22; C07D 491/048; C07D 271/10; C07D 261/12; C07D 295/192; C09K 15/30; C09K 15/28; C09K 15/20; C09K 15/06; C09K 15/22; C09K 15/12; C05C 3/00; C07C 271/44

(52) U.S. Cl. CPC C05G 3/08; C07C 271/44; C07C 255/54; C07C 333/08; C07C 217/84; C07D 249/14; C07D 513/04; C07C 43/215; C07C 271/58; C07D 241/24; C07C 233/25; C07D 241/18; C07C 251/48; C07D 257/04; C07D 249/12; C07C 275/34; C07C 205/37; C07D 233/86; C07D 401/12; C07D 213/73; C07C 255/65; C07C 243/22; C07C 69/92; C07C 243/28; C07C 251/76; C07C 49/255; C07C 47/575; C07D 277/68; C07C 43/23; C07D 253/075; C07C 335/18; C07D 307/68; C07D 495/04; C07C 311/37; C07D 239/34; C07D 209/34; C07D 295/205; C07D 231/56; C07D 277/54; C07D 211/16; C07D 275/04; C07D 487/04; C07D 239/545; C07D 401/04; C07D 239/42; C07D 275/03; C07D 261/20; C07D 311/22; C07D 491/048; C07D 271/10; C07D 261/12; C07D 295/192; C09K 15/30; C09K 15/28; C09K 15/20; C09K 15/06; C09K 15/22; C09K 15/12; C05C 3/00

(57) ABSTRACT

The present invention relates to novel nitrification inhibitors of formula I. Moreover, the invention relates to the use of these novel nitrification inhibitors for reducing nitrification, as well as agrochemical mixtures and compositions comprising the nitrification inhibitors. Further encompassed by the present invention are methods for reducing nitrification comprising the treatment of plants, soil and/or loci with said nitrification inhibitors, and methods for treating a fertilizer or a composition by applying said nitrification inhibitor.

The present invention relates to novel nitrification inhibitors of formula I. Moreover, the invention relates to the use of these compounds of formula I as nitrification inhibitors, i.e. for reducing nitrification, as well as agrochemical mixtures and compositions comprising the nitrification inhibitors. Further encompassed by the present invention are methods for reducing nitrification comprising the treatment of plants, soil and/or loci where the plant is growing or is intended to grow with said nitrification inhibitors and methods for treating a fertilizer or a composition by applying said nitrification inhibitor.

Nitrogen is an essential element for plant growth and reproduction. About 25% of the plant available nitrogen in soils (ammonium and nitrate) originate from decomposition processes (mineralization) of organic nitrogen compounds such as humus, plant and animal residues and organic fertilizers. Approximately 5% derive from rainfall. On a global basis, the biggest part (70%), however, is supplied to the plant by inorganic nitrogen fertilizers. The mainly used nitrogen fertilizers comprise ammonium compounds or derivatives thereof, i.e. nearly 90% of the nitrogen fertilizers applied worldwide is in the NH₄⁺ form (Subbarao et al., 2012, Advances in Agronomy, 114, 249-302). This is, inter alia, due to the fact that NH₄ assimilation is energetically more efficient than assimilation of other nitrogen sources such as NO₃⁻.

Moreover, being a cation, NH₄⁺ is held electrostatically by the negatively charged clay surfaces and functional groups of soil organic matter. This binding is strong enough to limit NH₄⁺-loss by leaching to groundwater. By contrast, NO₃⁻, being negatively charged, does not bind to the soil and is liable to be leached out of the plants' root zone. In addition, nitrate may be lost by denitrification which is the microbiological conversion of nitrate and nitrite (NO₂⁻) to gaseous forms of nitrogen such as nitrous oxide (N₂O) and molecular nitrogen (N₂).

However, ammonium (NH₄⁺) compounds are converted by soil microorganisms to nitrates (NO₃⁻) in a relatively short time in a process known as nitrification. The nitrification is carried out primarily by two groups of chemolithotrophic bacteria, ammonia-oxidizing bacteria (AOB) of the genus Nitrosomonas and Nitrobacter, which are ubiquitous component of soil bacteria populations. The enzyme, which is essentially responsible for nitrification is ammonia monooxygenase (AMO), which was also found in ammonia-oxidizing archaea (Subbarao et al., 2012, Advances in Agronomy, 114, 249-302).

The nitrification process typically leads to nitrogen leakage and environmental pollution. As a result of the various losses, approximately 50% of the applied nitrogen fertilizers is lost during the year following fertilizer addition (see Nelson and Huber; Nitrification inhibitors for corn production (2001), National Corn Handbook, Iowa State University).

As countermeasures the use of nitrification inhibitors, mostly together with fertilizers, was suggested. Suitable nitrification inhibitors include biological nitrification inhibitors (BNIs) such as linoleic acid, alpha-linolenic acid, methyl p-coumarate, methyl ferulate, MHPP, Karanjin, brachialacton or the p-benzoquinone sorgoleone (Subbarao et al., 2012, Advances in Agronomy, 114, 249-302). Further suitable nitrification inhibitors are synthetic chemical inhibitors such as Nitrapyrin, dicyandiamide (DCD), 3,4-dimethyl pyrazole phosphate (DMPP), 4-amino-1,2,4-triazole hydrochloride (ATC), 1-amido-2-thiourea (ASU), 2-amino-4-chloro-6-methylpyrimidine (AM), 5-ethoxy-3-trichloromethyl-1,2,4-thiodiazole (terrazole), or 2-sulfanilamidothiazole (ST) (Slangen and Kerkhoff, 1984, Fertilizer research, 5(1), 1-76).

EP 0 917 526 further mentions the use of polyacids to treat mineral fertilizers containing a nitrification inhibitor in order to improve the fixation of the nitrification inhibitors in the inorganic fertilizer. Moreover, the volatility of the nitrification inhibitor can be reduced.

However, many of these inhibitors only work sub-optimal. In addition, the world population is expected to grow significantly in the next 20-30 years, correspondingly food production in sufficient quantities and quality is necessary. In order to achieve this, the use of nitrogen fertilizers would have to double by 2050. For environmental reasons this is not possible, since nitrate levels in drinking water, eutrophication of surface water and gas emissions into the air have already reached critical levels in many places, causing water contamination and air pollution. However, less fertilizer would have to be used and fertilizer efficiency increases significantly if nitrification inhibitors are used. Therefore there is a clear need for novel nitrification inhibitors, as well as for methods using them.

In this connection, it is also desired to provide nitrification inhibitors, which are advantageous in terms of a reduced toxicity. It is therefore an object of the present invention to provide nitrification inhibitors, which have a satisfactory effectiveness as ntitrification inhibitors, but are less toxic than other nitrification inhibitors in the prior art. In particular, it is an object to provide nitrification inhibitors, which provide a reasonable balance between effectiveness and environmental safety.

A major disadvantage of a number of nitrification inhibitors including, e.g., pyrazole derivatives is their high volatility and susceptibility to hydrolysis. It is therefore another object to provide nitrification inhibitors, which are not volatile or susceptible to any degradation processes. Furthermore, it is an object of the present invention to provide nitrification inhibitors, which are not susceptible to leaching.

Furthermore, it is an object of the present invention to provide nitrification inhibitors, which act specifically as nitrification inhibitors, and e.g. do not act against other soil microorganisms than those involved in the nitrification process. In addition, the nitrification inhibitors should not have a negative effect on CH₄ oxidation of the soil.

Accordingly, there is a need for nitrification inhibitors, which are advantageous over the prior art in terms of at least one of the above objects.

The present invention addresses this need and relates to a novel nitrification inhibitor of formula I

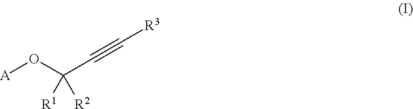

or a stereoisomer, salt, tautomer or N-oxide thereof,
wherein
A is aryl or hetaryl, wherein the aromatic ring may in each case be unsubstituted or may be partially or fully substituted by substituents, which are independently of each other selected from R^{A};
R¹ and R² are independently of each other selected from H and C₁-C₂-alkyl; and
R³ is H, C₁-C₄-haloalkyl, C₁-C₄-hydroxyalkyl, ethynylhydroxymethyl, phenylhydroxymethyl, or aryl, wherein the aromatic ring may be unsubstituted or may be partially or fully substituted by substituents, which are independently of each other selected from R^{B};
   and wherein
R^{A} is
   (i) halogen, CN, NR^{a}R^{b}, OR^{c}, SR^{c}, C(═Y¹)R^{c}, C(═Y¹)OR^{c}, C(═Y¹)SR^{c}, C(═Y¹)NR^{a}R^{b}, Y²C(═Y¹)R^{c}, Y²C(═Y¹)OR^{c}, Y²C(═Y¹)SR^{c}, Y²C(═Y¹)NR^{a}R^{b}, Y³Y²C(═Y¹)R^{c}, NR^{g}N═C(R^{d})(R^{e}), C(═N—OR^{c})R^{g}, C(═N—OR^{c})R^{g}, C(═N—SR^{c})R^{g}, C(═N—NR^{a}R^{b})R^{g}, S(═O)₂R^{f}, NR^{g}S(═O)₂R^{f}, S(═O)₂Y²C(═Y¹)R^{c}, S(═O)₂Y²C(═Y¹)OR^{c}, S(═O)₂Y²C(═Y¹)SR^{c}, S(═O)₂Y²C(═Y¹)NR^{a}R^{b}, NO₂, NON—CN, C₁-C₆-alkyl, C₂-C₆-alkenyl, C₂-C₆-alkynyl, C₁-C₄-haloalkyl, C₁-C₄-cyanoalkyl, C₁-C₄-hydroxyalkyl, C₁-C₄-alkoxy, C₂-C₄-alkynyl-C₁-C₂-hydroxyalkyl, C₂-C₄-alkynyloxy;
   (ii) C₁-C₄-alkylene-C(═Y¹)R^{c}, C₂-C₄-alkenylene-C(═Y¹)R^{c}, C₁-C₄-alkylene-C(═Y¹)OR^{c}, C₂-C₄-alkenylene-C(═Y¹)OR^{c}, C₁-C₄-alkylene-C(═Y¹)SR^{c}, C₂-C₄-alkenylene-C(═Y¹)SR^{c}, C₁-C₄-alkylene-C(═Y¹)NR^{a}NR^{b}, C₂-C₄-alkenylene-C(═Y¹)NR^{a}NR^{b}, C₁-C₄-alkylene-Y²—C(═Y¹)R^{c}, C₂-C₄-alkenylene-Y²—(═Y¹)R^{c}, C₁-C₄-alkylene-NR^{a}R^{b}, C₂-C₄-alkenylene-NR^{a}R^{b}, C₁-C₄-alkylene-OR^{c}, C₂-C₄-alkenylene-OR^{c}, C₁-C₄-alkylene-SR^{c}, C₂-C₄-alkenylene-SR^{c}, wherein the C₁-C₄-alkylene or C₂-C₄-alkenylene chain may in each case be unsubstituted or may be partially or fully substituted by OR^{g}, CN, halogen or phenyl;
   (iii) aryl, aryl-C₁-C₂-alkyl, hetaryl or hetaryl-C₁-C₂-alkyl, wherein the aromatic ring of the aryl or hetaryl group may be unsubstituted or may be partially or fully substituted by substituents, which are independently of each other selected from R^{h};
   (iv) a 3- to 14-membered saturated or unsaturated carbocycle or heterocycle, which may contain 1, 2, or 3 heteroatoms which, independently of each other, are selected from NR^{1b}, O, and S, wherein S may be oxidized and/or wherein the carbocycle or heterocycle may be unsubstituted or may be partially or fully substituted by substituents which, independently of each other, are selected from Rⁱ; and wherein R^{1b} is H, C₁-C₄-alkyl, C₂-C₄-alkenyl, C₃-C₆-cycloalkyl, C₃-C₆-cycloalkylmethyl, or OR^{g}; or
   (v) L-B, wherein
   L is —CH₂—, —CH═CH—, —C≡C—, —C(═O)— or —CH═, and
   B is aryl or hetaryl, wherein the aromatic ring of the ary or hetaryl group may be unsubstituted or may be partially or fully substituted by substituents, which are independently of each other selected from R^{h};
      or
      a 3- to 14-membered saturated or unsaturated carbocycle or heterocycle, which may contain 1, 2, or 3 heteroatoms which, independently of each other, are selected from NR^{1b}, O, and S, wherein S may be oxidized and/or wherein the carbocycle or heterocycle may be unsubstituted or may be partially or fully substituted by substituents which, independently of each other, are selected from Rⁱ; and wherein R^{1b} is H, C₁-C₄-alkyl, C₂-C₄-alkenyl, C₃-C₆-cycloalkyl, C₃-C₆-cycloalkylmethyl, or OR^{g}; or
   (vi) two substituents R^{A} together represent a carbocyclic or heterocyclic ring, which is fused to A and may contain 1, 2, or 3 heteroatoms which, independently of each other, are selected from NR^{1c}, O, and S, wherein S may be oxidized and/or wherein the carbocycle or heterocycle may be unsubstituted or may be partially or fully substituted by substituents which, independently of each other, are selected from Rⁱ; and wherein R^{1c} is H, C₁-C₄-alkyl, C₂-C₄-alkenyl, C₃-C₆-cycloalkyl, C₃-C₆-cycloalkylmethyl, C₃-C₆-heterocyclyl, C₃-C₆-heterocyclylmethyl or OR^{g};
      and wherein
R^{B} is NH—C(═O)—(C₁-C₄-alkyl), NH—C(═O)—(C₂-C₄-alkenyl), NH—C(═O)—(C₁-C₂-alkoxy-C₁-C₂-alkyl), NH—C(═O)—(C₃-C₆-cycloalkyl), NH—S(═O)₂—(C₁-C₄-alkyl), or NO₂;
   and wherein
Y¹, Y² and Y³ are independently of each other selected from O, S and NR^{1a}, wherein R^{1a} is in each case independently H, C₁-C₄-alkyl, C₂-C₄-alkenyl, C₃-C₆-cycloalkyl, C₃-C₆-cycloalkylmethyl, OR^{g}, SR^{g} or NR^{m}Rⁿ;
R^{a} and R^{b} are independently of each other selected from
   (i) H, NR^{j}R^{k}, OR^{l}, SR^{l}, C₁-C₄-alkyl, C₂-C₄-alkenyl, C₂-C₄-alkynyl, C₁-C₄-hydroxyalkyl, C₁-C₄-alkoxy, C(═Y¹)R^{l}, C(═Y¹)OR^{l}, C(═Y¹)SR^{l}, C(═Y¹)NR^{j}R^{k}, C(═Y¹)C(═Y²)R^{l}, S(═O)₂R^{f};
   (ii) aryl or hetaryl, wherein the aromatic ring of the aryl or hetaryl group may be unsubstituted or may be partially or fully substituted by substituents, which are independently of each other selected from R^{h}; or
R^{a} and R^{b} together with the nitrogen atom to which they are bound form
   (iii) a hetaryl group which may be unsubstituted or may be partially or fully substituted by substituents, which are independently of each other selected from R^{h}; or
   (iv) a 3- to 10-membered, saturated or unsaturated heterocycle, which may contain 1, 2, or 3 heteroatoms which, independently of each other, are selected from NR^{1b}, O, and S, wherein S may be oxidized and/or wherein the heterocycle may be unsubstituted or may be partially or fully substituted by substituents which, independently of each other, are selected from Rⁱ; and wherein R^{1b} is H, C₁-C₄-alkyl, C₂-C₄-alkenyl, C₃-C₆-cycloalkyl, C₃-C₆-cycloalkylmethyl, or OR^{g};
R^{c} is
   (i) H, C₁-C₄-alkyl, C₂-C₄-alkenyl, C₂-C₄-alkynyl, C(═O)OR^{l}, C(═O)SR^{l}, C(═O)NR^{j}R^{k};
   (ii) C₁-C₄-alkylene-C(═O)R^{l}, C₁-C₄-alkylene-C(═O)OR^{l}, wherein the C₁-C₄-alkylene chain may in each case be unsubstituted or may be partially or fully substituted by OR^{g}, CN, halogen, or phenyl;
   (iii) aryl, aryl-C₁-C₂-alkyl, hetaryl, or hetaryl-C₁-C₂-alkyl, wherein the aromatic ring of the aryl or hetaryl group may be unsubstituted or may be partially or fully substituted by substituents, which are independently of each other selected from R^{h}; or
   (iv) a 3- to 10-membered saturated or unsaturated carbocycle or heterocycle, which may contain 1, 2, or 3 heteroatoms which, independently of each other, are selected from NR^{1b}, O, and S, wherein S may be oxidized and/or wherein the carbocycle or heterocycle may be unsubstituted or may be partially or fully substituted by substituents which, independently of each other, are selected from Rⁱ; and wherein R^{1b} is H, C₁-C₄-alkyl, C₂-C₄-alkenyl, C₃-C₆-cycloalkyl, C₃-C₆-cycloalkylmethyl, or OR^{g};
R^{d} and R^{e} are independently selected from C₁-C₄-alkyl, C₁-C₄-haloalkyl, NR^{j}R^{k}, OR^{l}, SR^{l}, CN, C(═Y¹)R¹, C(═Y¹)OR^{l}, C(═Y¹)SR^{l}, or C(═Y)NR^{j}R^{k};
R^{f} is C₁-C₄-alkyl, C₁-C₄-haloalkyl, NR^{j}R^{k}, OR^{l}, SR^{l}, aryl or hetaryl, wherein the aromatic ring of the aryl or hetaryl group may be unsubstituted or may be partially or fully substituted by substituents, which are independently of each other selected from R^{h};
R^{g} is H or C₁-C₄-alkyl;
R^{h} is halogen, CN, NO₂, NR^{j}R^{k}, OR^{l}, SR^{l}, C₁-C₄-alkyl, C₂-C₄-alkenyl, C₂-C₄-alkynyl, C₁-C₄-haloalkyl, C₁-C₄-alkoxy, C₂-C₄-alkynyloxy, C(═Y¹)R^{l}, C(═Y¹)OR^{l}, C(═Y¹)SR^{l}, C(═Y¹)NR^{j}R^{k}, aryl, aryloxy, hetaryl and hetaryloxy;
Rⁱ is
   (i) halogen, CN, C₁-C₄-alkyl, C₂-C₄-alkenyl, C₂-C₄-alkynyl, C₁-C₄-haloalkyl, C₂-C₄-haloalkenyl;
   (ii)=NR^{1d}, wherein R^{1d} is H, C₁-C₄-alkyl, C₂-C₄-alkenyl, C₃-C₆-cycloalkyl, C₃-C₆-cycloalkylmethyl, or OR^{g};
   (iii) ═O, ═S, NR^{j}R^{k}, OR^{l}, SR^{l}, C(═Y¹)R^{l}, C(═Y¹)OR^{l}, C(═Y¹)SR^{l}, C(═Y¹)NR^{j}R^{k};
   (iv) aryl, aryl-C₁-C₂-alkyl, hetaryl, or hetaryl-C₁-C₂-alkyl, wherein the aromatic ring of the aryl or hetaryl group may be unsubstituted or may be partially or fully substituted by substituents, which are independently of each other selected from halogen, CN, C₁-C₄-alkyl, C₁-C₄-haloalkyl, C₁-C₄-alkoxy, C₂-C₄-alkynyloxy, OR^{g}, and SR^{g}; or
   (v) C₃-C₆-cycloalkyl, or 3- to 6-membered heterocyclyl, wherein the cycloalkyl ring or the heterocyclyl ring may be unsubstituted or may be partially or fully substituted by substituents, which are independently of each other selected from halogen, CN, C₁-C₄-alkyl, OR^{g}, and SR^{g};
   R^{j} and R^{k} are independently selected from H, OR^{g}, SR^{g}, C(═Y¹)R^{g}, C(═Y¹)OR^{g}, C(═Y¹)SR^{g}, C(═Y¹)NR^{m}Rⁿ, C₁-C₄-alkyl, C₂-C₄-alkenyl, C₂-C₄-alkynyl, C₁-C₄-haloalkyl, aryl or hetaryl, wherein the aromatic ring of the aryl or hetaryl group may be unsubstituted or may be partially or fully substituted by substituents, which are independently selected from halogen, CN, C₁-C₄-alkyl, C₁-C₄-haloalkyl, C₁-C₄-alkoxy, C₂-C₄-alkynyloxy, OR^{g}, and SR^{g};
Rⁱ is H, C₁-C₄-alkyl, C₂-C₄-alkenyl, C₂-C₄-alkynyl, C₁-C₄-haloalkyl, C(═Y¹)R^{g}, C(═Y¹)OR^{g}, C(═Y¹)SR^{g}, C(═Y¹)NR^{m}Rⁿ, aryl or hetaryl, wherein the aromatic ring of the aryl or hetaryl group may be unsubstituted or may be partially or fully substituted by substituents, which are independently selected from halogen, CN, C₁-C₄-alkyl, C₁-C₄-haloalkyl, C₁-C₄-alkoxy, C₂-C₄-alkynyloxy, OR^{g}, and SR^{g}; and
R^{m} and Rⁿ are independently selected from H and C₁-C₄-alkyl.

The inventors surprisingly found that by applying the compound of formula I as defined herein above the nitrification of ammonium to nitrate could significantly be reduced.

In one aspect, the present invention therefore relates to the use of the compounds of formula I as defined above as a nitrification inhibitor. In other words, the present invention relates to the use of the compounds of formula I as defined above for reducing nitrification.

Thus, in one aspect the present invention relates to the use of a nitrification inhibitor for reducing nitrification, wherein said nitrification inhibitor is a compound of formula I as defined herein above.

In a preferred embodiment of said use, in said compound of formula I, A is phenyl or a 5- or 6-membered hetaryl, wherein the aromatic ring may in each case be unsubstituted or may be partially or fully substituted by substituents, which are independently of each other selected from R^{A}. In a more preferred embodiment of said use, in said compound of formula I, A is phenyl or a 6-membered hetaryl, in particular phenyl, wherein the aromatic ring may in each case be unsubstituted or may be partially or fully substituted by substituents, which are independently of each other selected from R^{A}. In a most preferred embodiment, A is phenyl. Such compounds are typically solid at room temperature and are typically non-volatile and stable against hydrolysis.

In another preferred embodiment of said use, in said compound of formula I, R¹ and R² both represent hydrogen.

In yet another preferred embodiment of said use, in said compound of formula I, R³ is hydrogen, C₁-C₄-haloalkyl or ethinylhydroxymethyl, and preferably R³ is hydrogen.

In still another preferred embodiment of said use, in said compound of formula I, R^{A}, if present, is
(i) halogen, CN, NR^{a}R^{b}, OR^{c}, C(═Y¹)R^{c}, C(═Y¹)OR^{c}, C(═Y¹)SR^{c}, C(═Y¹)NR^{a}R^{b}, Y²C(═Y¹)R^{c}, Y²C(═Y¹)NR^{a}R^{b}, NR^{g}N═C(R^{d})(R^{e}), S(═O)₂R^{f}, NO₂, C₁-C₆-alkyl, C₂-C₆- C₁-C₄-haloalkyl, C₁-C₄-alkoxy, C₂-C₄-alkynyl-C₁-C₂-hydroxyalkyl, C₂-C₄-alkynyloxy;
(ii) C₂-C₄-alkenylene-C(═Y¹)R^{c}, C₂-C₄-alkenylene-Y²—C(═Y¹)R^{c}, wherein the C₁-C₄-alkylene or C₂-C₄-alkenylene chain may in each case be unsubstituted or may be partially or fully substituted by CN or halogen;
(iii) aryl, wherein the aromatic ring of the aryl group may be unsubstituted or may be partially or fully substituted by substituents, which are independently of each other selected from R^{h}; or
(iv) a 3- to 14-membered saturated or unsaturated heterocycle, which may contain 1, 2, or 3 heteroatoms which, independently of each other, are selected from NR^{1b}, O, and S, wherein S may be oxidized and/or wherein the heterocycle may be unsubstituted or may be partially or fully substituted by substituents which, independently of each other, are selected from Rⁱ; and wherein R^{1b} is H, C₁-C₄-alkyl, C₂-C₄-alkenyl, C₃-C₆-cycloalkyl, C₃-C₆-cycloalkylmethyl, or OR^{g},
   wherein preferably

Y¹, Y² and Y³ are independently of each other selected from O, S and NR^{1a}, wherein R^{1a} is in each case independently H, C₁-C₄-alkyl, OH, or NH₂.

R^{a} and R^{b} are independently of each other selected from
(i) H, NH₂, C₁-C₄-alkyl, C₁-C₄-hydroxyalkyl, C(═O)H, C(═S)H, C(═N—H)H, C(═N—(C₁-C₄)alkyl))H, C(═N—OH)H, C(═N—NH₂)H, or

R^{a} and R^{b} together with the nitrogen atom to which they are bound form
(iv) a 3- to 10-membered, saturated or unsaturated heterocycle, which may contain 1, 2, or 3 heteroatoms which, independently of each other, are selected from NR^{1b}, O, and S, wherein S may be oxidized and/or wherein the heterocycle may be unsubstituted or may be partially or fully substituted by substituents which, independently of each other, are selected from C₁-C₄-alkyl, C₁-C₄-haloalkyl, C₂-C₄-haloalkenyl, and ═O; and wherein R^{1b} is H, C₁-C₄-alkyl, or OH;

R^{c} is
(i) H, C₁-C₄-alkyl; or
(iv) a 3- to 10-membered saturated or unsaturated carbocycle or heterocycle, which may contain 1, 2, or 3 heteroatoms which, independently of each other, are selected from NR^{1b}, O, and S, wherein S may be oxidized and/or wherein the carbocycle or heterocycle may be unsubstituted or may be partially or fully substituted by substituents which, independently of each other, are selected from C₁-C₄-alkyl, C₁-C₄-haloalkyl, C₂-C₄-haloalkenyl, and ═O; and wherein R^{1b} is preferably H, C₁-C₄-alkyl, or OH;

R^{d} and R^{e} are independently selected from NH₂ and C(═O)OH;

R^{f} is C₁-C₄-alkyl;

R^{g} is H;

R^{h} is halogen or C₁-C₄-alkoxy;

and

Rⁱ is
(i) C₁-C₄-alkyl, C₁-C₄-haloalkyl, C₂-C₄-haloalkenyl; or
(iii) ═O.

In a particularly preferred embodiment of said use, in said compound of formula I,

R¹ and R² both represent hydrogen,

R³ is hydrogen, and

A is phenyl, wherein the aromatic ring is substituted by 1, 2, or 3 substituent(s) R^{A},

wherein the substituent(s) R^{A} are independently of each other selected from halogen, CN, NH₂, C(═O)NR^{a}R^{b}, NHC(═O)NR^{a}R^{b}, NHC(═S)NR^{a}R^{b}, NHC(═O)H, C₁-C₄-alkoxy, C₂-C₄-alkynyl-C₁-C₂-hydroxyalkyl, and C₂-C₄-alkynyloxy,

wherein R^{a} and R^{b} are in each case independently of each other selected from H, C₁-C₂-alkyl, NH₂, C₁-C₂-hydroxyalkyl, or wherein R^{a} and R^{b} may together with the nitrogen atom to which they are bonded form a morpholine ring.

In a further aspect the present invention relates to a composition for use in reducing nitrification, comprising at least one nitrification inhibitor as defined herein above and at least one carrier.

In a further aspect, the present invention relates to an agrochemical composition for use in reducing nitrification, comprising at least one nitrification inhibitor as defined above and at least one carrier.

In a further aspect the present invention relates to an agrochemical mixture comprising at least one fertilizer; and at least one nitrification inhibitor as defined as defined herein above; or at least one fertilizer and a composition as mentioned above, which may be used for reducing nitrification

In a preferred embodiment said nitrification inhibitor as defined above, i.e. the compound of formula I, is used for reducing nitrification in combination with a fertilizer. In a further specific embodiment said nitrification inhibitor as defined above is used for reducing nitrification in combination with a fertilizer in the form of an agrochemical mixture as mentioned above. In a further preferred embodiment said reduction of nitrification as mentioned above occurs in or on a plant, in the root zone of a plant, in or on soil or soil substituents and/or at the locus where a plant is growing or is intended to grow.

In another aspect, the present invention relates to a method for reducing nitrification, comprising treating a plant growing on soil or soil substituents and/or the locus or soil or soil substituents where the plant is growing or is intended to grow with at least one nitrification inhibitor as defined herein above, or with a composition as defined herein above, or with an agricultural composition as defined above. In a preferred embodiment of the method, the plant and/or the locus or soil or soil substituents where the plant is growing or is intended to grow is additionally provided with a fertilizer. In a further preferred embodiment of the method, the application of said nitrification inhibitor and of said fertilizer is carried out simultaneously or with a time lag. In a more preferred embodiment, said time lag is an interval of 1 day, 2 days, 3 days, 4 days, 5, days, 6 days, 1 week, 2 weeks or 3 weeks. In a particularly preferred embodiment, said time lag is an interval of 1 day, 2 days, 3 days, 1 week, 2 weeks or 3 weeks. In case of application with a time lag, a nitrification inhibitor as defined above may be applied first and then the fertilizer. In a further preferred embodiment of the method, in a first step a nitrification inhibitor as defined above is applied to seeds, to a plant and/or to the locus where the plant is growing or is intended to grow and in a second step the fertilizer is applied to a plant and/or to the locus where the plant is growing or is intended to grow, wherein the application of a said nitrification inhibitor in the first step and the fertilizer in the second step is carried out with a time lag of at least 1 day, 2 days, 3 days, 4 days, 5, days, 6 days, 1 week, 2 weeks or 3 weeks. In other embodiments of application with a time lag, a fertilizer as defined above may be applied first and then a nitrification inhibitor as defined above may be applied. In a further preferred embodiment of the method, in a first step a fertilizer is applied to a plant and/or to the locus where the plant is growing or is intended to grow and in a second step a nitrification inhibitor as defined above is applied to seeds, to a plant and/or to the locus where the plant is growing or is intended to grow, wherein the application of a said fertilizer in the first step and said nitrification inhibitor in the second step is carried out with a time lag of at least 1 day, 2 days, 3 days, 4 days, 5, days, 6 days, 1 week, 2 weeks or 3 weeks.

In a further aspect, the present invention relates to a method for treating a fertilizer or a composition, comprising the application of a nitrification inhibitor as defined herein above.

In one preferred embodiment of the use, agrochemical mixture or method of the invention, said fertilizer is a solid or liquid ammonium-containing inorganic fertilizer such as an NPK fertilizer, anhydrous ammonium, ammonium nitrate, calcium ammonium nitrate, ammonium sulfate nitrate, ammonium sulfate or ammonium phosphate; a solid or liquid organic fertilizer such as liquid manure, semi-liquid manure, stable manure, biogas manure and straw manure, worm castings, compost, seaweed or guano, or an urea-containing fertilizer such as, urea, formaldehyde urea, urea ammonium nitrate (UAN) solution, urea sulphur, stabilized urea, urea based NPK-fertilizers, or urea ammonium sulfate.

In another preferred embodiment of the use, agrochemical mixture or method of the invention, said fertilizer is an ammonium-containing inorganic fertilizer such as an NPK fertilizer, ammonium nitrate, calcium ammonium nitrate, ammonium sulfate nitrate, ammonium sulfate or ammonium phosphate; an organic fertilizer such as liquid manure, semi-liquid manure, stable manure, biogas manure and straw manure, worm castings, compost, seaweed or guano, or an urea-containing fertilizer such as, formaldehyde urea, UAN, urea sulphur, stabilized urea, urea based NPK-fertilizers, or urea ammonium sulfate.

In a further preferred embodiment of the use, agrochemical mixture or method of the invention, said plant is an agricultural plant such as wheat, barley, oat, rye, soybean, corn, potatoes, oilseed rape, canola, sunflower, cotton, sugar cane, sugar beet, rice or a vegetable such as spinach, lettuce, asparagus, or cabbages; or sorghum; a silvicultural plant; an ornamental plant; or a horticultural plant, each in its natural or in a genetically modified form.

The present invention relates in one aspect to the use of a nitrification inhibitor for reducing nitrification, wherein said nitrification inhibitor is a compound of formula I as defined herein.

Although the present invention will be described with respect to particular embodiments, this description is not to be construed in a limiting sense.

Before describing in detail exemplary embodiments of the present invention, definitions important for understanding the present invention are given. As used in this specification and in the appended claims, the singular forms of “a” and “an” also include the respective plurals unless the context clearly dictates otherwise. In the context of the present invention, the terms “about” and “approximately” denote an interval of accuracy that a person skilled in the art will understand to still ensure the technical effect of the feature in question. The term typically indicates a deviation from the indicated numerical value of ±20%, preferably ±15%, more preferably ±10%, and even more preferably ±5%. It is to be understood that the term “comprising” is not limiting. For the purposes of the present invention the term “consisting of” is considered to be a preferred embodiment of the term “comprising of”. If hereinafter a group is defined to comprise at least a certain number of embodiments, this is meant to also encompass a group which preferably consists of these embodiments only. Furthermore, the terms “first”, “second”, “third” or “(a)”, “(b)”, “(c)”, “(d)” etc. and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. In case the terms “first”, “second”, “third” or “(a)”, “(b)”, “(c)”, “(d)”, “i”, “ii” etc. relate to steps of a method or use or assay there is no time or time interval coherence between the steps, i.e. the steps may be carried out simultaneously or there may be time intervals of seconds, minutes, hours, days, weeks, months or even years between such steps, unless otherwise indicated in the application as set forth herein above or below. It is to be understood that this invention is not limited to the particular methodology, protocols, reagents etc. described herein as these may vary. It is also to be understood that the terminology used herein is for the purpose of describing particular embodiments only, and is not intended to limit the scope of the present invention that will be limited only by the appended claims. Unless defined otherwise, all technical and scientific terms used herein have the same meanings as commonly understood by one of ordinary skill in the art.

The term “nitrification inhibitor” is to be understood in this context as a chemical substance which slows down or stops the nitrification process. Nitrification inhibitors accordingly retard the natural transformation of ammonium into nitrate, by inhibiting the activity of bacteria such as Nitrosomonas spp. The term “nitrification” as used herein is to be understood as the biological oxidation of ammonia (NH₃) or ammonium (NH₄⁺) with oxygen into nitrite (NO₂⁻) followed by the oxidation of these nitrites into nitrates (NO₃⁻) by microorganisms. Besides nitrate (NO₃⁻) nitrous oxide is also produced through nitrification. Nitrification is an important step in the nitrogen cycle in soil. The inhibition of nitrification may thus also reduce N₂O losses. The term nitrification inhibitor is considered equivalent to the use of such a compound for inhibiting nitrification.

The compounds according to the invention are commercially available or can be prepared by standard processes and derivatizations known to a skilled person. For example, a phenyl propargyl ether can be prepared by reacting a phenol with propargylbromide or propargylchloride. Phenols with various substituents as well as various hydroxyhetaryl compounds, which may be used instead of phenols, are commercially available or can be easily prepared by a skilled person.

The term “compound(s) according to the invention”, or “compounds of formula I” comprises the compound(s) as defined herein as well as a stereoisomer, salt, tautomer or N-oxide thereof. The term “compound(s) of the present invention” is to be understood as equivalent to the term “compound(s) according to the invention”, therefore also comprising a stereoisomer, salt, tautomer or N-oxide thereof.

The compounds of formula I may be amorphous or may exist in one or more different crystalline states (polymorphs) which may have different macroscopic properties such as stability or show different biological properties such as activities. The present invention relates to amorphous and crystalline compounds of formula I, mixtures of different crystalline states of the respective compound I, as well as amorphous or crystalline salts thereof.

Salts of the compounds of the formula I are preferably agriculturally acceptable salts. They can be formed in a customary manner, e.g. by reacting the compound with an acid of the anion in question if the compound of formula I has a basic functionality. Agriculturally useful salts of the compounds of formula I encompass especially the acid addition salts of those acids whose cations and anions, respectively, have no adverse effect on the mode of action of the compounds of formula I. Anions of useful acid addition salts are primarily chloride, bromide, fluoride, hydrogensulfate, sulfate, dihydrogenphosphate, hydrogenphosphate, phosphate, nitrate, bicarbonate, carbonate, hexafluorosilicate, hexafluorophosphate, benzoate, and the anions of C₁-C₄-alkanoic acids, preferably formate, acetate, propionate and butyrate. They can be formed by reacting compounds of formula I with an acid of the corresponding anion, preferably of hydrochloric acid, hydrobromic acid, sulfuric acid, phosphoric acid or nitric acid.

The term “N-oxide” includes any compound of formula I which has at least one tertiary nitrogen atom that is oxidized to an N-oxide moiety.

The organic moieties mentioned in the above definitions of the variables are—like the term halogen—collective terms for individual listings of the individual group members. The prefix Cₙ-Cₘ indicates in each case the possible number of carbon atoms in the group.

The term “halogen” denotes in each case fluorine, bromine, chlorine or iodine, in particular fluorine, chlorine or bromine.

The term “alkyl” as used herein denotes in each case a straight-chain or branched alkyl group having usually from 1 to 10 carbon atoms, frequently from 1 to 6 carbon atoms, preferably 1 to 4 carbon atoms, more preferably from 1 to 3 carbon atoms. Examples of an alkyl group are methyl, ethyl, n-propyl, iso-propyl, n-butyl, 2-butyl, iso-butyl, tert-butyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, and 1-ethyl-2-methylpropyl.

The term “haloalkyl” as used herein denotes in each case a straight-chain or branched alkyl group having usually from 1 to 10 carbon atoms, frequently from 1 to 6 carbon atoms, preferably from 1 to 4 carbon atoms, wherein the hydrogen atoms of this group are partially or totally replaced with halogen atoms. Preferred haloalkyl moieties are selected from C₁-C₄-haloalkyl, more preferably from C₁-C₃-haloalkyl or C₁-C₂-haloalkyl, in particular from C₁-C₂-fluoroalkyl such as fluoromethyl, difluoromethyl, trifluoromethyl, 1-fluoroethyl, 2-fluoroethyl, 2,2-difluoroethyl, 2,2,2-trifluoroethyl, pentafluoroethyl, and the like.

The term “cyanoalkyl” as used herein denotes in each case a straight-chain or branched alkyl group having usually from 1 to 10 carbon atoms, frequently from 1 to 6 carbon atoms, preferably from 1 to 4 carbon atoms, wherein the hydrogen atoms of this group are partially or totally replaced with cyano groups. Preferred cyanoalkyl moieties are selected from C₁-C₄-cyanoalkyl, more preferably from C₁-C₃-cyanooalkyl or C₁-C₂-cyanoalkyl.

The term “hydroxyalkyl” as used herein denotes in each case a straight-chain or branched alkyl group having usually from 1 to 10 carbon atoms, frequently from 1 to 6 carbon atoms, preferably from 1 to 4 carbon atoms, wherein the hydrogen atoms of this group are partially or totally replaced with hydroxy groups. Preferred hydroxyalkyl moieties are selected from C₁-C₄-hydroxyalkyl, more preferably from C₁-C-hydroxyalkyl or C₁-C₂-hydroxyalkyl. Preferred hydroxyalkyl moieties are selected from hydroxymethyl, dihydroxymethyl, trihydroxymethyl, 1-hydroxyethyl and 2-hydroxyethyl.

The term “ethynylhydroxymethyl” as used herein refers to the following substituent.

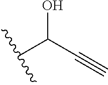 [SMILES: *C(O)C#C]

The term “phenylhydroxymethyl” as used herein refers to the following substituent.

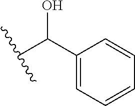 [SMILES: *C(O)c1ccccc1]

The term “alkoxy” as used herein denotes in each case a straight-chain or branched alkyl group which is bound via an oxygen atom and has usually from 1 to 10 carbon atoms, frequently from 1 to 6 carbon atoms, preferably 1 to 4 carbon atoms. Examples of an alkoxy group are methoxy, ethoxy, n-propoxy, iso-propoxy, n-butyloxy, 2-butyloxy, iso-butyloxy, tert-butyloxy, and the like.

The term “alkoxyalkyl” as used herein refers to alkyl usually comprising 1 to 10, frequently 1 to 4, preferably 1 to 2 carbon atoms, wherein 1 carbon atom carries an alkoxy radical usually comprising 1 to 4, preferably 1 or 2 carbon atoms as defined above. Examples are CH₂OCH₃, CH₂—OC₂H₅, 2-(methoxy)ethyl, and 2-(ethoxy)ethyl.

The term “alkylene” as used herein and in the moieties of e.g. C₁-C₄-alkylene-C(═Y¹)R^{c}, C₁-C₄-alkylene-C(═Y¹)OR^{c}, C₁-C₄-alkylene-C(═Y¹)SR^{c}, C₁-C₄-alkylene-C(═Y¹)NR^{a}NR^{b}, C₁-C₄-alkylene-Y²—C(═Y¹)R^{c}, C₁-C₄-alkylene-NR^{a}R^{b}, C₁-C₄-alkylene-OR^{c}, and C₁-C₄-alkylene-SR^{c} refers to a straight-chain or branched alkylene group having usually from 1 to 10 carbon atoms, frequently from 1 to 6 carbon atoms, preferably 1 to 4 carbon atoms, more preferably from 1 to 3 carbon atoms. Preferably, said alkenylene group connects a substituent, such as C(═Y¹)R^{c}, C(═Y¹)OR^{c}, C(═Y¹)SR^{c}, C(═Y¹)NR^{a}NR^{b}, Y²—C(═Y¹)R^{c}, NR^{a}R^{b}, OR^{c}, SR^{c}, with the remainder of the molecule.

The term “alkenyl” as used herein denotes in each case an at least singly unsaturated hydrocarbon radical, i.e. a hydrocarbon radical having at least one carbon-carbon double bond, having usually 2 to 10, frequently 2 to 6, preferably 2 to 4 carbon atoms, e.g. vinyl, allyl (2-propen-1-yl), 1-propen-1-yl, 2-propen-2-yl, methallyl (2-methylprop-2-en-1-yl), 2-buten-1-yl, 3-buten-1-yl, 2-penten-1-yl, 3-penten-1-yl, 4-penten-1-yl, 1-methylbut-2-en-1-yl, 2-ethylprop-2-en-1-yl and the like.

The term “haloalkenyl” as used herein refers to an alkenyl group as defined above, wherein the hydrogen atoms are partially or totally replaced with halogen atoms.

The term “alkenylene” as used herein and in the moieties of e.g. C₂-C₄-alkenylene-C(═Y¹)R^{c}, C₂-C₄-alkenylene-C(═Y¹)OR^{c}, C₂-C₄-alkenylene-C(═Y¹)SR^{c}, C₂-C₄-alkenylene-C(═Y¹)NR^{a}NR^{b}, C₂-C₄-alkenylene-Y²—C(═Y¹)R^{c}, C₂-C₄-alkenylene-NR^{a}R^{b}, C₂-C₄-alkenylene-OR^{c}, and C₂-C₄-alkenylene-SR^{c} refers to a straight-chain or branched alkenylene group, which is at least singly unsaturated, and has usually from 2 to 10 carbon atoms, frequently from 2 to 6 carbon atoms, preferably 2 to 4 carbon atoms, more preferably from 2 to 3 carbon atoms. Preferably, said alkenylene group connects a substituent, such as C(═Y¹)R^{c}, C(═Y¹)OR^{c}, C(═Y¹)SR^{c}, C(═Y¹)NR^{a}NR^{b}, Y²—C(═Y¹)R^{c}, NR^{a}R^{b}, OR^{c}, SR^{c}, with the remainder of the molecule.

The term “alkynyl” as used herein denotes in each case a hydrocarbon radical having at least one carbon-carbon triple bond and having usually 2 to 10, frequently 2 to 6, preferably 2 to 4 carbon atoms, e.g. ethynyl, propargyl (2-propyn-1-yl), 1-propyn-1-yl, 1-methylprop-2-yn-1-yl), 2-butyn-1-yl, 3-butyn-1-yl, 1-pentyn-1-yl, 3-pentyn-1-yl, 4-pentyn-1-yl, 1-methylbut-2-yn-1-yl, 1-ethylprop-2-yn-1-yl and the like.

The term “haloalkynyl” as used herein refers to an alkynyl group as defined above, wherein the hydrogen atoms are partially or totally replaced with halogen atoms.

The term “alkynylalkyl” as used herein refers to alkyl usually comprising 1 to 10, frequently 1 to 4, preferably 1 to 2 carbon atoms, wherein 1 carbon atom carries an alkynyl radical usually comprising 2 to 4, preferably 2 or 3 carbon atoms as defined above.

The term “alkynylhydroxyalkyl” as used herein refers to an hydroxyalkyl as defined above usually comprising 1 to 10, frequently 1 to 4, preferably 1 to 2 carbon atoms, wherein 1 carbon atom carries an alkynyl radical usually comprising 2 to 4, preferably 2 or 3 carbon atoms as defined above. An exemplary alkynylhydroxyalkyl is ethynylhydroxymethyl.

The term “alkynyloxy” as used herein denotes in each case an alkynyl group which is bound to the remainder of the molecule via an oxygen atom and has usually from 2 to 6 carbon atoms, frequently from 2 to 4 carbon atoms, preferably 2 to 3 carbon atoms.

The term “cycloalkyl” as used herein and in the cycloalkyl moieties of cycloalkoxy and cycloalkylmethyl denotes in each case a monocyclic cycloaliphatic radical having usually from 3 to 10 or from 3 to 6 carbon atoms, such as cyclopropyl, cyclobutyl, cyclopentyl, cyclohexyl, cycloheptyl, cyclooctyl, cyclononyl and cyclodecyl or cyclopropyl, cyclobutyl, cyclopentyl and cyclohexyl.

The term “cycloalkylalkyl” refers to a cycloalkyl group as defined above which is bound via an alkyl group, such as a C₁-C₅-alkyl group or a C₁-C₄-alkyl group, in particular a methyl group (=cycloalkylmethyl), to the remainder of the molecule.

The term “cycloalkenyl” as used herein denotes in each case a monocyclic singly unsaturated non-aromatic radical having usually from 5 to 10 or from 3 to 8 carbon atoms, including e.g. cycloheptenyl or cyclooctenyl.

The term “heterocyclyl” or “heterocycle” includes in general 3- to 14-membered, preferably 3- to 10-membered, more preferably 5-, or 6-membered non-aromatic radicals with at least one heteroatom. The term “heterocyclyl” or “heterocycle” refers to monocyclic, bicyclic or tricyclic heterocyclic non-aromatic radicals. The term “heterocyclyl” or “heterocycle” also includes bicyclic or tricyclic radicals, which comprise a non-aromatic ring and a fused aryl or hetaryl ring. Particularly preferred are 5- and 6-membered monocyclic heterocyclic non-aromatic radicals. The heterocyclic radicals usually comprise 1, 2, 3, 4 or 5, preferably 1, 2 or 3 heteroatoms selected from N, O and S as ring members, where S-atoms as ring members may be present as S, SO or SO₂. Examples of 5- or 6-membered heterocyclic radicals comprise saturated or unsaturated, non-aromatic heterocyclic rings, such as oxiranyl, oxetanyl, thietanyl, thietanyl-S-oxid (S-oxothietanyl), thietanyl-S-dioxid (S-dioxothiethanyl), pyrrolidinyl, pyrrolinyl, pyrazolinyl, tetrahydrofuranyl, dihydrofuranyl, 1,3-dioxolanyl, thiolanyl, S-oxothiolanyl, S-dioxothiolanyl, dihydrothienyl, S-oxodihydrothienyl, S-dioxodihydrothienyl, oxazolidinyl, oxazolinyl, thiazolinyl, oxathiolanyl, piperidinyl, piperazinyl, pyranyl, dihydropyranyl, tetrahydropyranyl, 1,3- and 1,4-dioxanyl, thiopyranyl, S.oxothiopyranyl, S-dioxothiopyranyl, dihydrothiopyranyl, S-oxodihydrothiopyranyl, S-dioxodihydrothiopyranyl, tetrahydrothiopyranyl, S-oxotetra-hydrothiopyranyl, S-dioxotetrahydrothiopyranyl, morpholinyl, thiomorpholinyl, S-oxothiomorpho-linyl, S-dioxothiomorpholinyl, thiazinyl and the like. Examples for heterocyclic rings also comprising 1 or 2 carbonyl groups as ring members comprise pyrrolidin-2-onyl, pyrrolidin-2,5-dionyl, imidazolidin-2-onyl, oxazolidin-2-onyl, thiazolidin-2-onyl and the like. Particularly preferred are also bicyclic 8- to 10-membered heterocyclic radicals comprising as ring members 1, 2, 3, 4 or 5, preferably 1, 2 or 3 heteroatoms selected from N, O and S members, where S-atoms as ring members may be present as S, SO or SO₂. Preferably, said bicyclic 8- to 10-membered heterocyclic radicals comprise a 5- or 6-membered non-aromatic ring as defined above, which is fused to an aryl or hetaryl ring or to another heterocyclic ring. These fused heterocyclyl radicals may be bound to the remainder of the molecule via any ring atom of the 5- or 6-membered ring or the fused ring.

The term “heterocyclylalkyl” refers to heterocyclyl as defined above, which is bound via a C₁-C₅-alkyl group or a C₁-C₄-alkyl group, in particular a methyl group (=heterocyclylmethyl), to the remainder of the molecule.

The term “carbocyclyl” or “carbocycle” includes in general 3- to 14-membered, preferably 3- to 10-membered, more preferably 5- or 6-membered non-aromatic radicals. The term “carbocyclyl” or “carbocycle” may refer to monocyclic, bicyclic or tricyclic carbocyclic non-aromatic radicals. Preferred carbocycles are cycloalkyl and cycloalkenyl groups having from 3 to 10, preferably 5 or 6 carbon atoms.

Particularly preferred are also bicyclic 8- to 10-membered carbocyclic radicals, wherein a 5- or 6-membered non-aromatic ring is fused to an aryl ring or another carbocyclic ring. These fused carbocyclyl radicals may be bonded to the remainder of the molecule via any ring atom of the 5- or 6-membered ring or the fused ring.

The term “carbocyclylalkyl” refers to carbocyclyl as defined above which is bound via a C₁-C₅-alkyl group or a C₁-C₄-alkyl group, in particular a methyl group (=carbocyclylmethyl), to the remainder of the molecule.

The term “hetaryl” includes monocyclic 5- or 6-membered heteroaromatic radicals comprising as ring members 1, 2, 3 or 4 heteroatoms selected from N, O and S. Examples of 5- or 6-membered heteroaromatic radicals include pyridyl, i.e. 2-, 3-, or 4-pyridyl, pyrimidinyl, i.e. 2-, 4- or 5-pyrimidinyl, pyrazinyl, pyridazinyl, i.e. 3- or 4-pyridazinyl, thienyl, i.e. 2- or 3-thienyl, furyl, i.e. 2- or 3-furyl, pyrrolyl, i.e. 2- or 3-pyrrolyl, oxazolyl, i.e. 2-, 3- or 5-oxazolyl, isoxazolyl, i.e. 3-, 4- or 5-isoxazolyl, thiazolyl, i.e. 2-, 3- or 5-thiazolyl, isothiazolyl, i.e. 3-, 4- or 5-isothiazolyl, pyrazolyl, i.e. 1-, 3-, 4- or 5-pyrazolyl, i.e. 1-, 2-, 4- or 5-imidazolyl, oxadiazolyl, e.g. 2- or 5-[1,3,4]oxadiazolyl, 4- or 5-(1,2,3-oxadiazol)yl, 3- or 5-(1,2,4-oxadiazol)yl, 2- or 5-(1,3,4-thiadiazol)yl, thiadiazolyl, e.g. 2- or 5-(1,3,4-thiadiazol)yl, 4- or 5-(1,2,3-thiadiazol)yl, 3- or 5-(1,2,4-thiadiazol)yl, triazolyl, e.g. 1H-, 2H- or 3H-1,2,3-triazol-4-yl, 2H-triazol-3-yl, 1H-, 2H-, or 4H-1,2,4-triazolyl and tetrazolyl, i.e. 1H- or 2H-tetrazolyl. The term “hetaryl” also includes bicyclic 8 to 13-membered, preferably 8- to 10 membered heteroaromatic radicals comprising as ring members 1, 2 or 3 heteroatoms selected from N, O and S, wherein a 5- or 6-membered heteroaromatic ring is fused to a phenyl ring or to a 5- or 6-membered heteroaromatic radical. Examples of a 5- or 6-membered heteroaromatic ring fused to a phenyl ring or to a 5- or 6-membered heteroaromatic radical include benzofuranyl, benzothienyl, indolyl, indazolyl, benzimidazolyl, benzoxathiazolyl, benzoxadiazolyl, benzothiadiazolyl, benzoxazinyl, chinolinyl, isochinolinyl, purinyl, 1,8-naphthyridyl, pteridyl, pyrido[3,2-d]pyrimidyl or pyridoimidazolyl and the like. These fused hetaryl radicals may be bonded to the remainder of the molecule via any ring atom of 5- or 6-membered heteroaromatic ring or via a carbon atom of the fused phenyl moiety.

The term “hetarylalkyl” refers to hetaryl as defined above which is bound via a C₁-C₅-alkyl group or a C₁-C₄-alkyl group, in particular a methyl group (=hetarylmethyl), to the remainder of the molecule.

The term “heteraryloxy” refers to heteraryl as defined above, which is bound via an oxygen atom to the remainder of the molecule.

The term “aryl” includes monocyclic, bicyclic or tricyclic aromatic radicals comprising 6 to 14 carbon atoms, preferably 6 or 10 carbon atoms, more preferably 6 carbon atoms. Exemplary aryl radicals include anthracenyl, naphthalenyl and phenyl. A preferred aryl radical is phenyl.

The term “arylalkyl” refers to aryl as defined above which is bound via a C₁-C₅-alkyl group or a C₁-C₄-alkyl group, in particular a methyl group (=arylmethyl), to the remainder of the molecule. A preferred arylalkyl group is phenylmethyl, i.e. benzyl.

The term “aryloxy” refers to aryl as defined above, which is bound via an oxygen atom to the remainder of the molecule. A preferred aryloxy group is e.g. benzyloxy.

As has been set out above, the present invention concerns in one aspect the use of a nitrification inhibitor for reducing nitrification, wherein said nitrification inhibitor is a compound of formula I

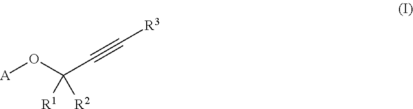

or a stereoisomer, salt, tautomer or N-oxide thereof,
wherein
A is aryl or hetaryl, wherein the aromatic ring may in each case be unsubstituted or may be partially or fully substituted by substituents, which are independently of each other selected from R^{A};
R¹ and R² are independently of each other selected from H and C₁-C₂-alkyl; and
R³ is H, C₁-C₄-haloalkyl, C₁-C₄-hydroxyalkyl, ethynylhydroxymethyl, phenylhydroxymethyl, or aryl, wherein the aromatic ring may be unsubstituted or may be partially or fully substituted by substituents, which are independently of each other selected from R^{B};
   and wherein
R^{A} is
   (i) halogen, CN, NR^{a}R^{b}, OR^{c}, SR^{c}, C(═Y¹)R^{c}, C(═Y¹)OR^{c}, C(═Y¹)SR^{c}, C(═Y¹)NR^{a}R^{b}, Y²C(═Y¹)R^{c}, Y²C(═Y¹)OR^{c}, Y²C(═Y¹)SR^{c}, Y²C(═Y¹)NR^{a}R^{b}, Y³Y²C(═Y¹)R^{c}, NR^{g}N═C(R^{d})(R^{e}), C(═N—OR^{c})R^{g}, C(═N—OR^{c})R^{g}, C(═N—SR^{c})R^{g}, C(═N—NR^{a}R^{b})R^{g}, S(═O)₂R^{f}, NR^{g}S(═O)₂R^{f}, S(═O)₂Y²C(═Y¹)R^{c}, S(═O)₂Y²C(═Y¹)OR^{c}, S(═O)₂Y²C(═Y¹)SR^{c}, S(═O)₂Y²C(═Y¹)NR^{a}R^{b}, NO₂, NON—CN, C₁-C₆-alkyl, C₂-C₆-alkenyl, C₂-C₆-alkynyl, C₁-C₄-haloalkyl, C₁-C₄-cyanoalkyl, C₁-C₄-hydroxyalkyl, C₁-C₄-alkoxy, C₂-C₄-alkynyl-C₁-C₂-hydroxyalkyl, C₂-C₄-alkynyloxy;
   (ii) C₁-C₄-alkylene-C(═Y¹)R^{c}, C₂-C₄-alkenylene-C(═Y¹)R^{c}, C₁-C₄-alkylene-C(═Y¹)OR^{c}, C₂-C₄-alkenylene-C(═Y¹)OR^{c}, C₁-C₄-alkylene-C(═Y¹)SR^{c}, C₂-C₄-alkenylene-C(═Y¹)SR^{c}, C₁-C₄-alkylene-C(═Y¹)NR^{a}NR^{b}, C₂-C₄-alkenylene-C(═Y¹)NR^{a}NR^{b}, C₁-C₄-alkylene-Y²—C(═Y¹)R^{c}, C₂-C₄-alkenylene-Y²—(═Y¹)R^{c}, C₁-C₄-alkylene-NR^{a}R^{b}, C₂-C₄-alkenylene-NR^{a}R^{b}, C₁-C₄-alkylene-OR^{c}, C₂-C₄-alkenylene-OR^{c}, C₁-C₄-alkylene-SR^{c}, C₂-C₄-alkenylene-SR^{c}, wherein the C₁-C₄-alkylene or C₂-C₄-alkenylene chain may in each case be unsubstituted or may be partially or fully substituted by OR^{g}, CN, halogen or phenyl;
   (iii) aryl, aryl-C₁-C₂-alkyl, hetaryl or hetaryl-C₁-C₂-alkyl, wherein the aromatic ring of the aryl or hetaryl group may be unsubstituted or may be partially or fully substituted by substituents, which are independently of each other selected from R^{h};
   (iv) a 3- to 14-membered saturated or unsaturated carbocycle or heterocycle, which may contain 1, 2, or 3 heteroatoms which, independently of each other, are selected from NR^{1b}, O, and S, wherein S may be oxidized and/or wherein the carbocycle or heterocycle may be unsubstituted or may be partially or fully substituted by substituents which, independently of each other, are selected from Rⁱ; and wherein R^{1b} is H, C₁-C₄-alkyl, C₂-C₄-alkenyl, C₃-C₆-cycloalkyl, C₃-C₆-cycloalkylmethyl, or OR^{g}; or
   (v) L-B, wherein
   L is —CH₂—, —CH═CH—, —C≡C—, —C(═O)— or —CH═, and
   B is aryl or hetaryl, wherein the aromatic ring of the ary or hetaryl group may be unsubstituted or may be partially or fully substituted by substituents, which are independently of each other selected from R^{h};
      or
      a 3- to 14-membered saturated or unsaturated carbocycle or heterocycle, which may contain 1, 2, or 3 heteroatoms which, independently of each other, are selected from NR^{1b}, O, and S, wherein S may be oxidized and/or wherein the carbocycle or heterocycle may be unsubstituted or may be partially or fully substituted by substituents which, independently of each other, are selected from Rⁱ; and wherein R^{1b} is H, C₁-C₄-alkyl, C₂-C₄-alkenyl, C₃-C₆-cycloalkyl, C₃-C₆-cycloalkylmethyl, or OR^{g}; or
   (vi) two substituents R^{A} together represent a carbocyclic or heterocyclic ring, which is fused to A and may contain 1, 2, or 3 heteroatoms which, independently of each other, are selected from NR^{1c}, O, and S, wherein S may be oxidized and/or wherein the carbocycle or heterocycle may be unsubstituted or may be partially or fully substituted by substituents which, independently of each other, are selected from Rⁱ; and wherein R^{1c} is H, C₁-C₄-alkyl, C₂-C₄-alkenyl, C₃-C₆-cycloalkyl, C₃-C₆-cycloalkylmethyl, C₃-C₆-heterocyclyl, C₃-C₆-heterocyclylmethyl or OR^{g};
      and wherein
R^{B} is NH—C(═O)—(C₁-C₄-alkyl), NH—C(═O)—(C₂-C₄-alkenyl), NH—C(═O)—(C₁-C₂-alkoxy-C₁-C₂-alkyl), NH—C(═O)—(C₃-C₆-cycloalkyl), NH—S(═O)₂—(C₁-C₄-alkyl), or NO₂;
   and wherein
Y¹, Y² and Y³ are independently of each other selected from O, S and NR^{1a}, wherein R^{1a} is in each case independently H, C₁-C₄-alkyl, C₂-C₄-alkenyl, C₃-C₆-cycloalkyl, C₃-C₆-cycloalkylmethyl, OR^{g}, SR^{g} or NR^{m}Rⁿ;
R^{a} and R^{b} are independently of each other selected from
   (i) H, NR^{j}R^{k}, OR^{l}, SR^{l}, C₁-C₄-alkyl, C₂-C₄-alkenyl, C₂-C₄-alkynyl, C₁-C₄-hydroxyalkyl, C₁-C₄-alkoxy, C(═Y¹)R^{l}, C(═Y¹)OR^{l}, C(═Y¹)SR^{l}, C(═Y¹)NR^{j}R^{k}, C(═Y¹)C(═Y²)R^{l}, S(═O)₂R^{f};
   (ii) aryl or hetaryl, wherein the aromatic ring of the aryl or hetaryl group may be unsubstituted or may be partially or fully substituted by substituents, which are independently of each other selected from R^{h}; or
R^{a} and R^{b} together with the nitrogen atom to which they are bound form
   (iii) a hetaryl group which may be unsubstituted or may be partially or fully substituted by substituents, which are independently of each other selected from R^{h}; or
   (iv) a 3- to 10-membered, saturated or unsaturated heterocycle, which may contain 1, 2, or 3 heteroatoms which, independently of each other, are selected from NR^{1b}, O, and S, wherein S may be oxidized and/or wherein the heterocycle may be unsubstituted or may be partially or fully substituted by substituents which, independently of each other, are selected from Rⁱ; and wherein R^{1b} is H, C₁-C₄-alkyl, C₂-C₄-alkenyl, C₃-C₆-cycloalkyl, C₃-C₆-cycloalkylmethyl, or OR^{g};
R^{c} is
   (i) H, C₁-C₄-alkyl, C₂-C₄-alkenyl, C₂-C₄-alkynyl, C(═O)OR^{l}, C(═O)SR^{l}, C(═O)NR^{j}R^{k};
   (ii) C₁-C₄-alkylene-C(═O)R^{l}, C₁-C₄-alkylene-C(═O)OR^{l}, wherein the C₁-C₄-alkylene chain may in each case be unsubstituted or may be partially or fully substituted by OR^{g}, CN, halogen, or phenyl;
   (iii) aryl, aryl-C₁-C₂-alkyl, hetaryl, or hetaryl-C₁-C₂-alkyl, wherein the aromatic ring of the aryl or hetaryl group may be unsubstituted or may be partially or fully substituted by substituents, which are independently of each other selected from R^{h}; or
   (iv) a 3- to 10-membered saturated or unsaturated carbocycle or heterocycle, which may contain 1, 2, or 3 heteroatoms which, independently of each other, are selected from NR^{1b}, O, and S, wherein S may be oxidized and/or wherein the carbocycle or heterocycle may be unsubstituted or may be partially or fully substituted by substituents which, independently of each other, are selected from Rⁱ; and wherein R^{1b} is H, C₁-C₄-alkyl, C₂-C₄-alkenyl, C₃-C₆-cycloalkyl, C₃-C₆-cycloalkylmethyl, or OR^{g};
R^{d} and R^{e} are independently selected from C₁-C₄-alkyl, C₁-C₄-haloalkyl, NR^{j}R^{k}, OR^{l}, SR^{l}, CN, C(═Y¹)R^{l}, C(═Y¹)OR^{l}, C(═Y¹)SR^{l}, or C(═Y¹)NR^{j}R^{k};
R^{f} is C₁-C₄-alkyl, C₁-C₄-haloalkyl, NR^{j}R^{k}, OR^{l}, SR^{l}, aryl or hetaryl, wherein the aromatic ring of the aryl or hetaryl group may be unsubstituted or may be partially or fully substituted by substituents, which are independently of each other selected from R^{h};
R^{g} is H or C₁-C₄-alkyl;
R^{h} is halogen, CN, NO₂, NR^{j}R^{k}, OR^{l}, SR^{l}, C₁-C₄-alkyl, C₂-C₄-alkenyl, C₂-C₄-alkynyl, C₁-C₄-haloalkyl, C₁-C₄-alkoxy, C₂-C₄-alkynyloxy, C(═Y¹)R^{l}, C(═Y¹)OR^{l}, C(═Y¹)SR^{l}, C(═Y¹)NR^{j}R^{k}, aryl, aryloxy, hetaryl and hetaryloxy;
Rⁱ is
   (i) halogen, CN, C₁-C₄-alkyl, C₂-C₄-alkenyl, C₂-C₄-alkynyl, C₁-C₄-haloalkyl, C₂-C₄-haloalkenyl;
   (ii) ═NR^{d}, wherein R^{1d} is H, C₁-C₄-alkyl, C₂-C₄-alkenyl, C₃-C₆-cycloalkyl, C₃-C₆-cycloalkylmethyl, or OR^{g};
   (iii) ═O, ═S, NR^{j}R^{k}, OR^{l}, SR^{l}, C(═Y¹)R^{l}, C(═Y¹)OR^{l}, C(═Y¹)SR^{l}, C(═Y¹)NR^{j}R^{k};
   (iv) aryl, aryl-C₁-C₂-alkyl, hetaryl, or hetaryl-C₁-C₂-alkyl, wherein the aromatic ring of the aryl or hetaryl group may be unsubstituted or may be partially or fully substituted by substituents, which are independently of each other selected from halogen, CN, C₁-C₄-alkyl, C₁-C₄-haloalkyl, C₁-C₄-alkoxy, C₂-C₄-alkynyloxy, OR^{g}, and SR^{g}; or
   (v) C₃-C₆-cycloalkyl, or 3- to 6-membered heterocyclyl, wherein the cycloalkyl ring or the heterocyclyl ring may be unsubstituted or may be partially or fully substituted by substituents, which are independently of each other selected from halogen, CN, C₁-C₄-alkyl, OR^{g}, and SR^{g};
R^{j} and R^{k} are independently selected from H, OR^{g}, SR^{g}, C(═Y¹)R^{g}, C(═Y¹)OR^{g}, C(═Y¹)SR^{g}, C(═Y¹)NR^{m}Rⁿ, C₁-C₄-alkyl, C₂-C₄-alkenyl, C₂-C₄-alkynyl, C₁-C₄-haloalkyl, aryl or hetaryl, wherein the aromatic ring of the aryl or hetaryl group may be unsubstituted or may be partially or fully substituted by substituents, which are independently selected from halogen, CN, C₁-C₄-alkyl, C₁-C₄-haloalkyl, C₁-C₄-alkoxy, C₂-C₄-alkynyloxy, OR^{g}, and SR^{g};
R^{l} is H, C₁-C₄-alkyl, C₂-C₄-alkenyl, C₂-C₄-alkynyl, C₁-C₄-haloalkyl, C(═Y¹)R^{g}, C(═Y¹)OR^{g}, C(═Y¹)SR^{g}, C(═Y¹)NR^{m}Rⁿ, aryl or hetaryl, wherein the aromatic ring of the aryl or hetaryl group may be unsubstituted or may be partially or fully substituted by substituents, which are independently selected from halogen, CN, C₁-C₄-alkyl, C₁-C₄-haloalkyl, C₁-C₄-alkoxy, C₂-C₄-alkynyloxy, OR^{g}, and SR^{g}; and
R^{m} and Rⁿ are independently selected from H and C₁-C₄-alkyl.

In other words, the present invention relates to the use of a compound of formula I as defined herein for reducing nitrification.

As already indicated above, the present invention also relates to compositions and agricultural mixtures comprising the compounds of formula I and to methods comprising the application of compounds of formula I. Preferred embodiments regarding the compounds of formula I, which are relevant for the use according to the present invention as well as to the compositions, mixtures and methods according to the present invention, are described in the following.

In a preferred embodiment of said compound of formula I, A is phenyl or a 5- or 6-membered hetaryl, wherein the aromatic ring may in each case be unsubstituted or may be partially or fully substituted by substituents, which are independently of each other selected from R^{A}. These compounds correspond to compounds of formula I.1, wherein A′ represents phenyl or a 5- or 6-membered hetaryl, wherein the aromatic ring may in each case be unsubstituted or may be partially or fully substituted by substituents, which are independently of each other selected from R^{A}.

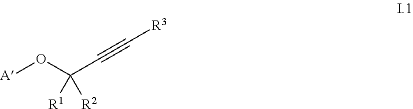

It can be preferred in terms of a reduced volatility and/or toxicity of the compounds of formula I.1, if A′ is not a pyrazole moiety. If A′ in the compounds of formula I.1 is a 5-membered hetaryl, it is preferred that said 5-membered hetaryl is tetrazole, thiazole or isothiazole, in particular isothiazole. If a A′ in the compounds of formula I.1 is a 6-membered hetaryl, it is preferred that said 6-membered hetaryl is pyridine, 1,3-diazine or 1,4-diazine.

In a more preferred embodiment of said compound of formula I, A is phenyl or a 6-membered hetaryl, wherein the aromatic ring may in each case be unsubstituted or may be partially or fully substituted by substituents, which are independently of each other selected from R^{A}. These compounds correspond to compounds of formula I.2, wherein A″ represents phenyl or a 6-membered hetaryl, wherein the aromatic ring may in each case be unsubstituted or may be partially or fully substituted by substituents, which are independently of each other selected from R^{A}.

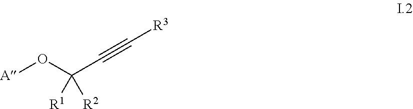

If a A″ in the compounds of formula I.2 is a 6-membered hetaryl, it is preferred that said 6-membered hetaryl is pyridine, 1,3-diazine or 1,4-diazine.

In an even more preferred embodiment of said compound of formula I, A is phenyl, wherein the aromatic ring may be unsubstituted or may be partially or fully substituted by substituents, which are independently of each other selected from R^{A}. These compounds correspond to compounds of formula I.3, wherein A′″ represents phenyl, wherein the aromatic ring may in each case be unsubstituted or may be partially or fully substituted by substituents, which are independently of each other selected from R^{A}.

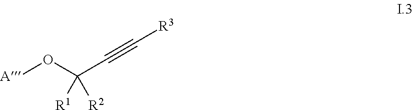

It has been found that the compounds of formula I, wherein A is phenyl, typically have high melting points, so that they are solid at room temperature and non-volatile. Furthermore, the compounds typically exhibit a low susceptibility to hydrolysis.

In a further preferred embodiment of said compound of formula I, R¹ and R² both represent hydrogen. These compounds correspond to compounds of formula I.A.

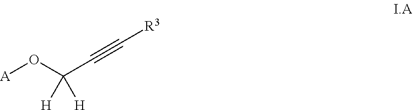

In another preferred embodiment of said compound of formula I, R³ is hydrogen, C₁-C₄-haloalkyl, ethynylhydroxymethyl, or phenylhydroxymethyl. It is more preferred that R³ is hydrogen, C₁-C₄-haloalkyl or ethynylhydroxymethyl. It is even more preferred that R³ is hydrogen. These compounds correspond to compounds of formula I.X.

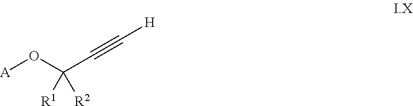

In a particularly preferred embodiment of said compound of formula I, A is A′, i.e. phenyl or a 5- or 6-membered hetaryl, wherein the aromatic ring may in each case be unsubstituted or may be partially or fully substituted by substituents, which are independently of each other selected from R^{A}, and R¹ and R² are both hydrogen. These compounds correspond to compounds of formula I.1.A.

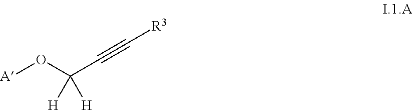

If A′ in the compounds of formula I.1.A is a 5-membered hetaryl, it is preferred that said 5-membered hetaryl is tetrazole, thiazole or isothiazole, in particular isothiazole. If a A′ in the compounds of formula I.1.A is a 6-membered hetaryl, it is preferred that said 6-membered hetaryl is pyridine, 1,3-diazine or 1,4-diazine.

In a more preferred embodiment of said compound of formula I, A is A″, i.e. phenyl or a 6-membered hetaryl, wherein the aromatic ring may in each case be unsubstituted or may be partially or fully substituted by substituents, which are independently of each other selected from R^{A}, and R¹ and R² are both hydrogen. These compounds correspond to compounds of formula I.2.A.

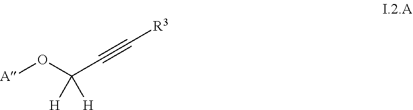

If a A″ in the compounds of formula I.2.A is a 6-membered hetaryl, it is preferred that said 6-membered hetaryl is pyridine, 1,3-diazine or 1,4-diazine.

In an even more preferred embodiment of said compound of formula I, A is A′″, i.e. phenyl, wherein the aromatic ring may in each case be unsubstituted or may be partially or fully substituted by substituents, which are independently of each other selected from R^{A}, and R¹ and R² are both hydrogen. These compounds correspond to compounds of formula I.3.A.

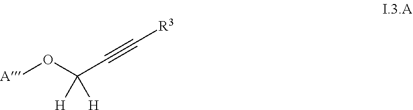

In another particularly preferred embodiment of said compound of formula I, A is A′, i.e. phenyl or a 5- or 6-membered hetaryl, wherein the aromatic ring may in each case be unsubstituted or may be partially or fully substituted by substituents, which are independently of each other selected from R^{A}, and R³ is hydrogen. These compounds correspond to compounds of formula I.1.X.

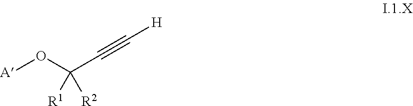

If A′ in the compounds of formula I.1.X is a 5-membered hetaryl, it is preferred that said 5-membered hetaryl is tetrazole, thiazole or isothiazole, in particular isothiazole. If a A′ in the compounds of formula I.1.X is a 6-membered hetaryl, it is preferred that said 6-membered hetaryl is pyridine, 1,3-diazine or 1,4-diazine.

In a more preferred embodiment of said compound of formula I, A is A″, i.e. phenyl or a 6-membered hetaryl, wherein the aromatic ring may in each case be unsubstituted or may be partially or fully substituted by substituents, which are independently of each other selected from R^{A}, and R³ is hydrogen. These compounds correspond to compounds of formula I.2.X.

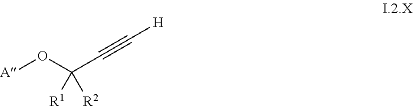

If a A″ in the compounds of formula I.2.X is a 6-membered hetaryl, it is preferred that said 6-membered hetaryl is pyridine, 1,3-diazine or 1,4-diazine.

In an even more preferred embodiment of said compound of formula I, A is A′″, i.e. phenyl, wherein the aromatic ring may in each case be unsubstituted or may be partially or fully substituted by substituents, which are independently of each other selected from R^{A}, and R³ is hydrogen. These compounds correspond to compounds of formula I.3.X.

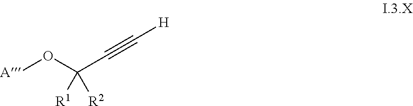

In another particularly preferred embodiment of said compound of formula I, R¹ and R² are both hydrogen, and R³ is hydrogen. These compounds correspond to compounds of formula I.A.X.

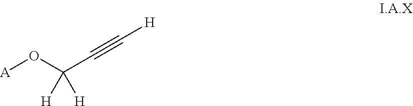

In a particularly preferred embodiment of said compound of formula I, A is A′, i.e. phenyl or a 5- or 6-membered hetaryl, wherein the aromatic ring may in each case be unsubstituted or may be partially or fully substituted by substituents, which are independently of each other selected from R^{A}, R¹ and R² are both hydrogen, and R³ is hydrogen. These compounds correspond to compounds of formula I.1.A.X.

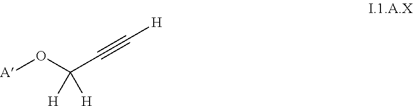

If A′ in the compounds of formula I.1.A.X is a 5-membered hetaryl, it is preferred that said 5-membered hetaryl is tetrazole, thiazole or isothiazole, in particular isothiazole. If a A′ in the compounds of formula I.1.A.X is a 6-membered hetaryl, it is preferred that said 6-membered hetaryl is pyridine, 1,3-diazine or 1,4-diazine.

In a more preferred embodiment of said compound of formula I, A is A″, i.e. phenyl or a 6-membered hetaryl, wherein the aromatic ring may in each case be unsubstituted or may be partially or fully substituted by substituents, which are independently of each other selected from R^{A}, R¹ and R² are both hydrogen, and R³ is hydrogen. These compounds correspond to compounds of formula I.2.A.X.

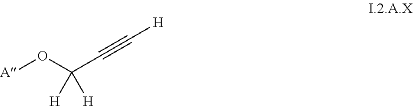

If a A″ in the compounds of formula I.2.A.X is a 6-membered hetaryl, it is preferred that said 6-membered hetaryl is pyridine, 1,3-diazine or 1,4-diazine.

In the most preferred embodiment of said compound of formula I, A is A′″, i.e. phenyl, wherein the aromatic ring may in each case be unsubstituted or may be partially or fully substituted by substituents, which are independently of each other selected from R^{A}, R¹ and R² are both hydrogen, and R³ is hydrogen. These compounds correspond to compounds of formula I.3.A.X.

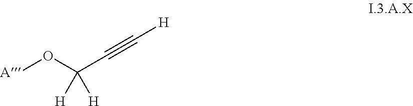

It has been found that the phenylpropargylether core structure of the compounds of formula I.3.A.X is particularly advantageous as compounds based on this core structure have a low volatility and exhibit a low susceptibility to hydrolysis. At the same time, they effectively inhibit nitrification.

In connection with compounds of formula I being compounds of formula I.3.A.X as defined above, it is preferred that the aromatic ring is substituted by one, two, or three substituents R^{A}. In one especially preferred embodiment, the compound of formula I therefore is a compound of formula I.3.A.X.(R^{A})₁, which corresponds to a compound of formula I.3.A.X as defined above, wherein the aromatic ring is substituted by one substituent R^{A}. In another especially preferred embodiment, the compound of formula I therefore is a compound of formula I.3.A.X.(R^{A})₂, which corresponds to a compound of formula I.3.A.X as defined above, wherein the aromatic ring is substituted by two substituents R^{A}. In yet another especially preferred embodiment, the compound of formula I therefore is a compound of formula I.3.A.X.(R^{A})₃, which corresponds to a compound of formula I.3.A.X as defined above, wherein the aromatic ring is substituted by three substituents R^{A}.

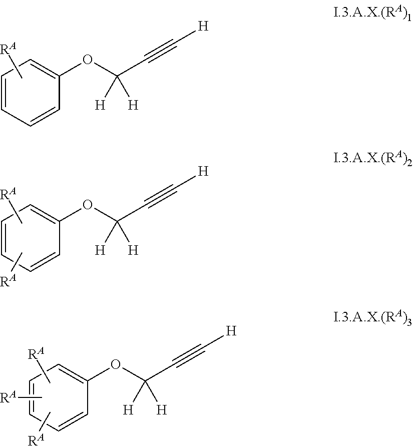

In relation to the above depicted compounds of formulae I.3.A.X.(R^{A})₁, I.3.A.X.(R^{A})₂, and I.3.A.X.(R^{A})₃, it is to be understood that the one, two or three R^{A} substituent(s) may be present at any carbon atom of the aromatic ring. Furthermore, it is to be understood that the substituents R^{A} may be identical or different in case of the compounds of formulae I.3.A.X.(R^{A})₂ and I.3.A.X.(R^{A})₃.

Further preferred embodiments of the compounds of formula I relate to the substituents R^{A}, with which A may be partially or fully substituted.

In a preferred embodiment of the compound of formula I, R^{A}, if present, is
(i) halogen, CN, NR^{a}R^{b}, OR^{c}, C(═Y¹)R^{c}, C(═Y¹)OR^{c}, C(═Y¹)SR^{c}, C(═Y¹)NR^{a}R^{b}, Y²C(═Y¹)R^{c}, Y²C(═Y¹)NR^{a}R^{b}, NR^{g}N═C(R^{d})(R^{e}), S(═O)₂R^{f}, NO₂, C₁-C₆-alkyl, C₂-C₆- C₁-C₄-haloalkyl, C₁-C₄-alkoxy, C₂-C₄-alkynyl-C₁-C₂-hydroxyalkyl, C₂-C₄-alkynyloxy;
(ii) C₂-C₄-alkenylene-C(═Y¹)R^{c}, C₂-C₄-alkenylene-Y²—(═Y¹)R^{c}, wherein the C₁-C₄-alkylene or C₂-C₄-alkenylene chain may in each case be unsubstituted or may be partially or fully substituted by CN or halogen;
(iii) aryl, wherein the aromatic ring of the aryl group may be unsubstituted or may be partially or fully substituted by substituents, which are independently of each other selected from R^{h}; or
(iv) a 3- to 14-membered saturated or unsaturated heterocycle, which may contain 1, 2, or 3 heteroatoms which, independently of each other, are selected from NR^{1b}, O, and S, wherein S may be oxidized and/or wherein the heterocycle may be unsubstituted or may be partially or fully substituted by substituents which, independently of each other, are selected from Rⁱ; and wherein R^{1b} is H, C₁-C₄-alkyl, C₂-C₄-alkenyl, C₃-C₆-cycloalkyl, C₃-C₆-cycloalkylmethyl, or OR^{g}.

In a particularly preferred embodiment of the present invention, the compound of formula I is a compound of formula I.1, I.A, I.X, I.1.A, I.1.X, I.A.X or I.1.A.X, wherein R^{A}, if present, is
(i) halogen, CN, NR^{a}R^{b}, OR^{c}, C(═Y¹)R^{c}, C(═Y¹)OR^{c}, C(═Y¹)SR^{c}, C(═Y¹)NR^{a}R^{b}, Y²C(═Y¹)R^{c}, Y²C(═Y¹)NR^{a}R^{b}, NR^{g}N═C(R^{d})(R^{e}), S(═O)₂R^{f}, NO₂, C₁-C₆-alkyl, C₂-C₆- C₁-C₄-haloalkyl, C₁-C₄-alkoxy, C₂-C₄-alkynyl-C₁-C₂-hydroxyalkyl, C₂-C₄-alkynyloxy;
(ii) C₂-C₄-alkenylene-C(═Y¹)R^{c}, C₂-C₄-alkenylene-Y²—(═Y¹)R^{c}, wherein the C₁-C₄-alkylene or C₂-C₄-alkenylene chain may in each case be unsubstituted or may be partially or fully substituted by CN or halogen;
(iii) aryl, wherein the aromatic ring of the aryl group may be unsubstituted or may be partially or fully substituted by substituents, which are independently of each other selected from R^{h}; or
(iv) a 3- to 14-membered saturated or unsaturated heterocycle, which may contain 1, 2, or 3 heteroatoms which, independently of each other, are selected from NR^{1b}, O, and S, wherein S may be oxidized and/or wherein the heterocycle may be unsubstituted or may be partially or fully substituted by substituents which, independently of each other, are selected from Rⁱ; and wherein R^{1b} is H, C₁-C₄-alkyl, C₂-C₄-alkenyl, C₃-C₆-cycloalkyl, C₃-C₆-cycloalkylmethyl, or OR^{g}.

In another particularly preferred embodiment of the present invention, the compound of formula I is a compound of formula I.2, I.3, I.2.A, I.3.A, I.2.X, I.3.X, I.2.A.X, or I.3.A.X, especially a compound of formula I.3.A.X, wherein R^{A}, if present, is
(i) halogen, CN, NR^{a}R^{b}, OR^{c}, C(═Y¹)R^{c}, C(═Y¹)OR^{c}, C(═Y¹)SR^{c}, C(═Y¹)NR^{a}R^{b}, Y²C(═Y¹)R^{c}, Y²C(═Y¹)NR^{a}R^{b}, NR^{g}N═C(R^{d})(R^{e}), S(═O)₂R^{f}, NO₂, C₁-C₆-alkyl, C₁-C₄-haloalkyl, C₁-C₄-alkoxy, C₂-C₄-alkynyl-C₁-C₂-hydroxyalkyl, C₂-C₄-alkynyloxy;
(ii) C₂-C₄-alkenylene-C(═Y¹)R^{c}, C₂-C₄-alkenylene-Y²—(═Y¹)R^{c}, wherein the C₁-C₄-alkylene or C₂-C₄-alkenylene chain may in each case be unsubstituted or may be partially or fully substituted by CN or halogen;
(iii) aryl, wherein the aromatic ring of the aryl group may be unsubstituted or may be partially or fully substituted by substituents, which are independently of each other selected from R^{h}; or
(iv) a 3- to 14-membered saturated or unsaturated heterocycle, which may contain 1, 2, or 3 heteroatoms which, independently of each other, are selected from NR^{1b}, O, and S, wherein S may be oxidized and/or wherein the heterocycle may be unsubstituted or may be partially or fully substituted by substituents which, independently of each other, are selected from Rⁱ; and wherein R^{1b} is H, C₁-C₄-alkyl, C₂-C₄-alkenyl, C₃-C₆-cycloalkyl, C₃-C₆-cycloalkylmethyl, or OR^{g}.

In another particularly preferred embodiment of the present invention, the compound of formula I is a compound of formula I.3.A.X.(R^{A})₁, I.3.A.X.(R^{A})₂, or I.3.A.X.(R^{A})₃, wherein R^{A} is
(i) halogen, CN, NR^{a}R^{b}, OR^{c}, C(═Y¹)R^{c}, C(═Y¹)OR^{c}, C(═Y¹)SR^{c}, C(═Y¹)NR^{a}R^{b}, Y²C(═Y¹)R^{c}, Y²C(═Y¹)NR^{a}R^{b}, NR^{g}N═C(R^{d})(R^{e}), S(═O)₂R^{f}, NO₂, C₁-C₆-alkyl, C₁-C₄-haloalkyl, C₁-C₄-alkoxy, C₂-C₄-alkynyl-C₁-C₂-hydroxyalkyl, C₂-C₄-alkynyloxy;
(ii) C₂-C₄-alkenylene-C(═Y¹)R^{c}, C₂-C₄-alkenylene-Y²—(═Y¹)R^{c}, wherein the C₁-C₄-alkylene or C₂-C₄-alkenylene chain may in each case be unsubstituted or may be partially or fully substituted by CN or halogen;
(iii) aryl, wherein the aromatic ring of the aryl group may be unsubstituted or may be partially or fully substituted by substituents, which are independently of each other selected from R^{h}; or
(iv) a 3- to 14-membered saturated or unsaturated heterocycle, which may contain 1, 2, or 3 heteroatoms which, independently of each other, are selected from NR^{1b}, O, and S, wherein S may be oxidized and/or wherein the heterocycle may be unsubstituted or may be partially or fully substituted by substituents which, independently of each other, are selected from Rⁱ; and wherein R^{1b} is H, C₁-C₄-alkyl, C₂-C₄-alkenyl, C₃-C₆-cycloalkyl, C₃-C₆-cycloalkylmethyl, or OR^{g}.

In one particularly preferred embodiment, the compounds of formula I are compounds of formula I.3.A.X, in particular compounds of formula I.3.A.X.(R^{A})₁, I.3.A.X.(R^{A})₂, or I.3.A.X.(R^{A})₃, wherein R^{A} is selected from the substituents listed under option (i) above. In another particularly preferred embodiment, the compounds of formula I are compounds of formula I.3.A.X, in particular compounds of formula I.3.A.X.(R^{A})₁, I.3.A.X.(R^{A})₂, or I.3.A.X.(R^{A})₃, wherein R^{A} is selected from the substituents listed under option (ii) above. In yet another particularly preferred embodiment, the compounds of formula I are compounds of formula I.3.A.X, in particular compounds of formula I.3.A.X.(R^{A})₁, I.3.A.X.(R^{A})₂, or I.3.A.X.(R^{A})₃, wherein R^{A} is selected from the substituents listed under option (iii) above. In yet another particularly preferred embodiment, the compounds of formula I are compounds of formula I.3.A.X, in particular compounds of formula I.3.A.X.(R^{A})₁, I.3.A.X.(R^{A})₂, or I.3.A.X.(R^{A})₃, wherein R^{A} is selected from the substituents listed under option (iv) above.

In one especially preferred embodiment, the compounds of formula I are compounds of formula I.3.A.X, in particular compounds of formula I.3.A.X.(R^{A})₁, I.3.A.X.(R^{A})₂, or I.3.A.X.(R^{A})₃, wherein R^{A} is selected from the substituents listed under option (i) above.

If R^{A} is present in the compounds of formula I or a compound of any one of formulae I.1, I.A, I.X, I.1.A, I.1.X, I.A.X or I.1.A.X, and preferably if R^{A} is selected as indicated above, the following substituent definitions are particularly preferred according to the present invention.

If R^{A} is present in a compound of any one of formulae I.2, I.3, I.2.A, I.3.A, I.2.X, I.3.X, I.2.A.X, or I.3.A.X, and preferably if R^{A} is selected as indicated above, the following substituent definitions are particularly preferred according to the present invention.

Especially, if R^{A} is present in the compounds of formula I.3.A.X, and if R^{A} is selected from the preferred substituents defined above under options (i), (ii), (iii) and (iv), the following substituent definitions are preferred according to the present invention.

The following substituent definitions are also particularly preferred in connection with the compounds of formula I.3.A.X.(R^{A})₁, I.3.A.X.(R^{A})₂, and I.3.A.X.(R^{A})₃, if R^{A} is selected from the preferred substituents defined above under options (i), (ii), (iii) and (iv).

Preferably, Y¹, Y² and Y³ are independently of each other selected from O, S and NR^{1a}, wherein preferably R^{1a} is in each case independently H, C₁-C₄-alkyl, OR^{g}, or NR^{m}Rⁿ, wherein more preferably R^{1a} is in each case independently H, C₁-C₄-alkyl, OH, or NH₂.

Preferably, R^{a} and R^{b} are independently of each other selected from
(i) H, NR^{j}R^{k}, C₁-C₄-alkyl, C₁-C₄-hydroxyalkyl, C(═Y¹)R^{l}; or

R^{a} and R^{b} together with the nitrogen atom to which they are bound form
(iv) a 3- to 10-membered, saturated or unsaturated heterocycle, which may contain 1, 2, or 3 heteroatoms which, independently of each other, are selected from NR^{1b}, O, and S, wherein S may be oxidized and/or wherein the heterocycle may be unsubstituted or may be partially or fully substituted by substituents which, independently of each other, are selected from Rⁱ; and wherein R^{1b} is preferably H, C₁-C₄-alkyl, or OR^{g}.

More preferably, R^{a} and R^{b} are independently of each other selected from
(i) H, NH₂, C₁-C₄-alkyl, C₁-C₄-hydroxyalkyl, C(═O)H, C(═S)H, C(═N—H)H, C(═N—(C₁-C₄)alkyl))H, C(═N—OH)H, C(═N—NH₂)H; or

R^{a} and R^{b} together with the nitrogen atom to which they are bound form
(iv) a 3- to 10-membered, saturated or unsaturated heterocycle, which may contain 1, 2, or 3 heteroatoms which, independently of each other, are selected from NR^{1b}, O, and S, wherein S may be oxidized and/or wherein the heterocycle may be unsubstituted or may be partially or fully substituted by substituents which, independently of each other, are selected from C₁-C₄-alkyl, C₁-C₄-haloalkyl, C₂-C₄-haloalkenyl, and ═O; and wherein R^{1b} is H, C₁-C₄-alkyl, or OH.

Most preferably, R^{a} and R^{b} are independently of each other selected from
(i) H, NH₂, C₁-C₄-alkyl, and C₁-C₄-hydroxyalkyl; or

R^{a} and R^{b} together with the nitrogen atom to which they are bound form
(iv) morpholine, piperidine, or piperazin.

Preferably, R^{c} is
(i) H, C₁-C₄-alkyl; or
(iv) a 3- to 10-membered saturated or unsaturated carbocycle or heterocycle, which may contain 1, 2, or 3 heteroatoms which, independently of each other, are selected from NR^{1b}, O, and S, wherein S may be oxidized and/or wherein the carbocycle or heterocycle may be unsubstituted or may be partially or fully substituted by substituents which, independently of each other, are selected from Rⁱ; and wherein R^{1b} is preferably H, C₁-C₄-alkyl, or OR^{g}.

More preferably, R^{c} is
(i) H, C₁-C₄-alkyl; or
(iv) a 3- to 10-membered saturated or unsaturated carbocycle or heterocycle, which may contain 1, 2, or 3 heteroatoms which, independently of each other, are selected from NR^{1b}, O, and S, wherein S may be oxidized and/or wherein the carbocycle or heterocycle may be unsubstituted or may be partially or fully substituted by substituents which, independently of each other, are selected from C₁-C₄-alkyl, C₁-C₄-haloalkyl, C₂-C₄-haloalkenyl, and ═O; and wherein R^{1b} is H, C₁-C₄-alkyl, or OH.

Preferably, R^{d} and R^{e} are independently selected from NR^{j}R^{k} and C(═Y¹)OR^{l}.

More preferably, R^{d} and R^{e} are independently selected from NH₂ and C(═O)OH.

Preferably, R^{f} is C₁-C₄-alkyl.

Preferably, R^{g} is H.

Preferably, R^{h} is halogen or C₁-C₄-alkoxy.

Preferably, R^{l} is
(i) C₁-C₄-alkyl, C₁-C₄-haloalkyl, C₂-C₄-haloalkenyl; or
(iii) ═O.

Preferably, R^{j} and R^{k} are both H.

Preferably, R^{l} is H.

Preferably, R^{m} and Rⁿ are both H.

Preferred compounds of formula I are compounds of formula I.1, I.A, I.X, I.1.A, I.1.X, I.A.X or I.1.A.X, or compounds of formula I.2, I.3, I.2.A, I.3.A, I.2.X, I.3.X, I.2.A.X, or I.3.A.X, wherein R^{A}, if present, is
(i) halogen, CN, NR^{a}R^{b}, OR^{c}, C(═Y¹)R^{c}, C(═Y¹)OR^{c}, C(═Y¹)SR^{c}, C(═Y¹)NR^{a}R^{b}, Y²C(═Y¹)R^{c}, Y²C(═Y¹)NR^{a}R^{b}, NR^{g}N═C(R^{d})(R^{e}), S(═O)₂R^{f}, NO₂, C₁-C₆-alkyl, C₁-C₄-haloalkyl, C₁-C₄-alkoxy, C₂-C₄-alkynyl-C₁-C₂-hydroxyalkyl, C₂-C₄-alkynyloxy;
(ii) C₂-C₄-alkenylene-C(═Y¹)R^{c}, C₂-C₄-alkenylene-Y²—(═Y¹)R^{c}, wherein the C₁-C₄-alkylene or C₂-C₄-alkenylene chain may in each case be unsubstituted or may be partially or fully substituted by CN or halogen;
(iii) aryl, wherein the aromatic ring of the aryl group may be unsubstituted or may be partially or fully substituted by substituents, which are independently of each other selected from R^{h}; or
(iv) a 3- to 14-membered saturated or unsaturated heterocycle, which may contain 1, 2, or 3 heteroatoms which, independently of each other, are selected from NR^{1b}, O, and S, wherein S may be oxidized and/or wherein the heterocycle may be unsubstituted or may be partially or fully substituted by substituents which, independently of each other, are selected from Rⁱ; and wherein R^{1b} is H, C₁-C₄-alkyl, C₂-C₄-alkenyl, C₃-C₆-cycloalkyl, C₃-C₆-cycloalkylmethyl, or OR^{g},

and wherein

Y¹, Y² and Y³ are independently of each other selected from O, S and NR^{1a}, wherein preferably R^{1a} is in each case independently H, C₁-C₄-alkyl, OR^{g}, or NR^{m}Rⁿ. and wherein more preferably R^{1a} is in each case independently H, C₁-C₄-alkyl, OH, or NH₂.

and wherein

R^{a} and R^{b} are independently of each other selected from
(i) H, NR^{j}R^{k}, C₁-C₄-alkyl, C₁-C₄-hydroxyalkyl, C(═Y¹)R^{l}; or

R^{a} and R^{b} together with the nitrogen atom to which they are bound form
(iv) a 3- to 10-membered, saturated or unsaturated heterocycle, which may contain 1, 2, or 3 heteroatoms which, independently of each other, are selected from NR^{1b}, O, and S, wherein S may be oxidized and/or wherein the heterocycle may be unsubstituted or may be partially or fully substituted by substituents which, independently of each other, are selected from Rⁱ; and wherein R^{1b} is preferably H, C₁-C₄-alkyl, or OR^{g},

and wherein

R^{c} is
(i) H, C₁-C₄-alkyl; or
(iv) a 3- to 10-membered saturated or unsaturated carbocycle or heterocycle, which may contain 1, 2, or 3 heteroatoms which, independently of each other, are selected from NR^{1b}, O, and S, wherein S may be oxidized and/or wherein the carbocycle or heterocycle may be unsubstituted or may be partially or fully substituted by substituents which, independently of each other, are selected from Rⁱ; and wherein R^{1b} is preferably H, C₁-C₄-alkyl, or OR^{g};

and wherein

R^{d} and R^{e} are independently selected from NR^{j}R^{k} and C(═Y¹)OR^{l};

and wherein

R^{f} is C₁-C₄-alkyl;

and wherein

R^{g} is H;

and wherein

R^{h} is halogen or C₁-C₄-alkoxy;

and wherein

Rⁱ is
(i) C₁-C₄-alkyl, C₁-C₄-haloalkyl, C₂-C₄-haloalkenyl; or
(iii) ═O;

and wherein

R^{j} and R^{k} are both H;

and wherein

R^{l} is H;

and wherein

R^{m} and Rⁿ are both H.

More preferred compounds of formula I are compounds of formula I.1, I.A, I.X, I.1.A, I.1.X, I.A.X or I.1.A.X, or compounds of formula I.2, I.3, I.2.A, I.3.A, I.2.X, I.3.X, I.2.A.X, or I.3.A.X, wherein R^{A}, if present, is
(i) halogen, CN, NR^{a}R^{b}, OR^{c}, C(═Y¹)R^{c}, C(═Y¹)OR^{c}, C(═Y¹)SR^{c}, C(═Y¹)NR^{a}R^{b}, Y²C(═Y¹)R^{c}, Y²C(═Y¹)NR^{a}R^{b}, NR^{g}N═C(R^{d})(R^{e}), S(═O)₂R^{f}, NO₂, C₁-C₆-alkyl, C₁-C₄-haloalkyl, C₁-C₄-alkoxy, C₂-C₄-alkynyl-C₁-C₂-hydroxyalkyl, C₂-C₄-alkynyloxy;
(ii) C₂-C₄-alkenylene-C(═Y¹)R^{c}, C₂-C₄-alkenylene-Y²—(═Y¹)R^{c}, wherein the C₁-C₄-alkylene or C₂-C₄-alkenylene chain may in each case be unsubstituted or may be partially or fully substituted by CN or halogen;
(iii) aryl, wherein the aromatic ring of the aryl group may be unsubstituted or may be partially or fully substituted by substituents, which are independently of each other selected from R^{h}; or
(iv) a 3- to 14-membered saturated or unsaturated heterocycle, which may contain 1, 2, or 3 heteroatoms which, independently of each other, are selected from NR^{1b}, O, and S, wherein S may be oxidized and/or wherein the heterocycle may be unsubstituted or may be partially or fully substituted by substituents which, independently of each other, are selected from Rⁱ; and wherein R^{1b} is H, C₁-C₄-alkyl, C₂-C₄-alkenyl, C₃-C₆-cycloalkyl, C₃-C₆-cycloalkylmethyl, or OR^{g},

and wherein

Y¹, Y² and Y³ are independently of each other selected from O, S and NR^{1a}, wherein R^{1a} is in each case independently H, C₁-C₄-alkyl, OH, or NH₂.

and wherein

R^{a} and R^{b} are independently of each other selected from
(i) H, NH₂, C₁-C₄-alkyl, C₁-C₄-hydroxyalkyl, C(═O)H, C(═S)H, C(═N—H)H, C(═N—(C₁-C₄)alkyl))H, C(═N—OH)H, C(═N—NH₂)H, or

R^{a} and R^{b} together with the nitrogen atom to which they are bound form
(iv) a 3- to 10-membered, saturated or unsaturated heterocycle, which may contain 1, 2, or 3 heteroatoms which, independently of each other, are selected from NR^{1b}, O, and S, wherein S may be oxidized and/or wherein the heterocycle may be unsubstituted or may be partially or fully substituted by substituents which, independently of each other, are selected from C₁-C₄-alkyl, C₁-C₄-haloalkyl, C₂-C₄-haloalkenyl, and ═O; and wherein R^{1b} is H, C₁-C₄-alkyl, or OH,

and wherein

R^{c} is
(i) H, C₁-C₄-alkyl; or
(iv) a 3- to 10-membered saturated or unsaturated carbocycle or heterocycle, which may contain 1, 2, or 3 heteroatoms which, independently of each other, are selected from NR^{1b}, O, and S, wherein S may be oxidized and/or wherein the carbocycle or heterocycle may be unsubstituted or may be partially or fully substituted by substituents which, independently of each other, are selected from C₁-C₄-alkyl, C₁-C₄-haloalkyl, C₂-C₄-haloalkenyl, and ═O; and wherein R^{1b} is preferably H, C₁-C₄-alkyl, or OH;

and wherein

R^{d} and R^{e} are independently selected from NH₂ and C(═O)OH;

R^{f} is C₁-C₄-alkyl;

and wherein

R^{g} is H;

and wherein

R^{h} is halogen or C₁-C₄-alkoxy;

and wherein

Rⁱ is
(i) C₁-C₄-alkyl, C₁-C₄-haloalkyl, C₂-C₄-haloalkenyl; or
(iii) ═O.

More preferred compounds of formula I are compounds of formula I.1, I.A, I.X, I.1.A, I.1.X, I.A.X or I.1.A.X, or compounds of formula I.2, I.3, I.2.A, I.3.A, I.2.X, I.3.X, I.2.A.X, or I.3.A.X, wherein the substituent(s) R^{A}, if present, are independently of each other selected from halogen, CN, NH₂, C(═O)NR^{a}R^{b}, NHC(═O)NR^{a}R^{b}, NHC(═S)NR^{a}R^{b}, NHC(═O)H, C₁-C₄-alkoxy, C₂-C₄-alkynyl-C₁-C₂-hydroxyalkyl, and C₂-C₄-alkynyloxy, wherein R^{a} and R^{b} are in each case independently selected from H, C₁-C₂-alkyl, NH₂, C₁-C₂-hydroxyalkyl, or wherein R^{a} and R^{b} may together with the nitrogen atom to which they are bonded form a morpholine ring.

In view of the above, particularly preferred compounds of formula I are compounds of formula I.3.A.X, in particular compounds of formula I.3.A.X.(R^{A})₁, I.3.A.X.(R^{A})₂, and I.3.A.X.(R^{A})₃, wherein the substituent(s) R^{A} are independently of each other selected from
(i) halogen, CN, NR^{a}R^{b}, OR^{c}, C(═Y¹)R^{c}, C(═Y¹)OR^{c}, C(═Y¹)SR^{c}, C(═Y¹)NR^{a}R^{b}, Y²C(═Y¹)R^{c}, Y²C(═Y¹)NR^{a}R^{b}, NR^{g}N═C(R^{d})(R^{e}), S(═O)₂R^{f}, NO₂, C₁-C₆-alkyl, C₁-C₄-haloalkyl, C₁-C₄-alkoxy, C₂-C₄-alkynyl-C₁-C₂-hydroxyalkyl, C₂-C₄-alkynyloxy;
(ii) C₂-C₄-alkenylene-C(═Y¹)R^{c}, C₂-C₄-alkenylene-Y²—(═Y¹)R^{c}, wherein the C₁-C₄-alkylene or C₂-C₄-alkenylene chain may in each case be unsubstituted or may be partially or fully substituted by CN or halogen;
(iii) aryl, wherein the aromatic ring of the aryl group may be unsubstituted or may be partially or fully substituted by substituents, which are independently of each other selected from R^{h}; or
(iv) a 3- to 14-membered saturated or unsaturated heterocycle, which may contain 1, 2, or 3 heteroatoms which, independently of each other, are selected from NR^{1b}, O, and S, wherein S may be oxidized and/or wherein the heterocycle may be unsubstituted or may be partially or fully substituted by substituents which, independently of each other, are selected from Rⁱ; and wherein R^{1b} is H, C₁-C₄-alkyl, C₂-C₄-alkenyl, C₃-C₆-cycloalkyl, C₃-C₆-cycloalkylmethyl, or OR^{g},

and wherein

Y¹, Y² and Y³ are independently of each other selected from O, S and NR^{1a}, wherein preferably R^{1a} is in each case independently H, C₁-C₄-alkyl, OR^{g}, or NR^{m}Rⁿ. and wherein more preferably R^{1a} is in each case independently H, C₁-C₄-alkyl, OH, or NH₂.

and wherein

R^{a} and R^{b} are independently of each other selected from
(i) H, NR^{j}R^{k}, C₁-C₄-alkyl, C₁-C₄-hydroxyalkyl, C(═Y¹)R^{l}; or

R^{a} and R^{b} together with the nitrogen atom to which they are bound form
(iv) a 3- to 10-membered, saturated or unsaturated heterocycle, which may contain 1, 2, or 3 heteroatoms which, independently of each other, are selected from NR^{1b}, O, and S, wherein S may be oxidized and/or wherein the heterocycle may be unsubstituted or may be partially or fully substituted by substituents which, independently of each other, are selected from Rⁱ; and wherein R^{1b} is preferably H, C₁-C₄-alkyl, or OR^{g},

and wherein

R^{c} is
(i) H, C₁-C₄-alkyl; or
(iv) a 3- to 10-membered saturated or unsaturated carbocycle or heterocycle, which may contain 1, 2, or 3 heteroatoms which, independently of each other, are selected from NR^{1b}, O, and S, wherein S may be oxidized and/or wherein the carbocycle or heterocycle may be unsubstituted or may be partially or fully substituted by substituents which, independently of each other, are selected from Rⁱ; and wherein R^{1b} is preferably H, C₁-C₄-alkyl, or OR^{g};

and wherein

R^{d} and R^{e} are independently selected from NR^{j}R^{k} and C(═Y¹)OR^{l};

and wherein

R^{f} is C₁-C₄-alkyl;

and wherein

R^{g} is H;

and wherein

R^{h} is halogen or C₁-C₄-alkoxy;

and wherein

Rⁱ is
(i) C₁-C₄-alkyl, C₁-C₄-haloalkyl, C₂-C₄-haloalkenyl; or
(iii) ═O;

and wherein

R^{j} and R^{k} are both H;

and wherein

R^{l} is H;

and wherein

R^{m} and Rⁿ are both H.

In view of the above, especially preferred compounds of formula I are compounds of formula I.3.A.X, in particular compounds of formula I.3.A.X.(R^{A})₁, I.3.A.X.(R^{A})₂, and I.3.A.X.(R^{A})₃, wherein the substituent(s) R^{A} are independently of each other selected from
(i) halogen, CN, NR^{a}R^{b}, OR^{c}, C(═Y¹)R^{c}, C(═Y¹)OR^{c}, C(═Y¹)SR^{c}, C(═Y¹)NR^{a}R^{b}, Y²C(═Y¹)R^{c}, Y²C(═Y¹)NR^{a}R^{b}, NR^{g}N═C(R^{d})(R^{e}), S(═O)₂R^{f}, NO₂, C₁-C₆-alkyl, C₁-C₄-haloalkyl, C₁-C₄-alkoxy, C₂-C₄-alkynyl-C₁-C₂-hydroxyalkyl, C₂-C₄-alkynyloxy;
(ii) C₂-C₄-alkenylene-C(═Y¹)R^{c}, C₂-C₄-alkenylene-Y²—(═Y¹)R^{c}, wherein the C₁-C₄-alkylene or C₂-C₄-alkenylene chain may in each case be unsubstituted or may be partially or fully substituted by CN or halogen;
(iii) aryl, wherein the aromatic ring of the aryl group may be unsubstituted or may be partially or fully substituted by substituents, which are independently of each other selected from R^{h}; or
(iv) a 3- to 14-membered saturated or unsaturated heterocycle, which may contain 1, 2, or 3 heteroatoms which, independently of each other, are selected from NR^{1b}, O, and S, wherein S may be oxidized and/or wherein the heterocycle may be unsubstituted or may be partially or fully substituted by substituents which, independently of each other, are selected from Rⁱ; and wherein R^{1b} is H, C₁-C₄-alkyl, C₂-C₄-alkenyl, C₃-C₆-cycloalkyl, C₃-C₆-cycloalkylmethyl, or OR^{g},

and wherein

Y¹, Y² and Y³ are independently of each other selected from O, S and NR^{1a}, wherein R^{1a} is in each case independently H, C₁-C₄-alkyl, OH, or NH₂.

and wherein

R^{a} and R^{b} are independently of each other selected from
(i) H, NH₂, C₁-C₄-alkyl, C₁-C₄-hydroxyalkyl, C(═O)H, C(═S)H, C(═N—H)H, C(═N—(C₁-C₄)alkyl))H, C(═N—OH)H, C(═N—NH₂)H, or

R^{a} and R^{b} together with the nitrogen atom to which they are bound form
(iv) a 3- to 10-membered, saturated or unsaturated heterocycle, which may contain 1, 2, or 3 heteroatoms which, independently of each other, are selected from NR^{1b}, O, and S, wherein S may be oxidized and/or wherein the heterocycle may be unsubstituted or may be partially or fully substituted by substituents which, independently of each other, are selected from C₁-C₄-alkyl, C₁-C₄-haloalkyl, C₂-C₄-haloalkenyl, and ═O; and wherein R^{1b} is H, C₁-C₄-alkyl, or OH,

and wherein

R^{c} is
(i) H, C₁-C₄-alkyl; or
(iv) a 3- to 10-membered saturated or unsaturated carbocycle or heterocycle, which may contain 1, 2, or 3 heteroatoms which, independently of each other, are selected from NR^{1b}, O, and S, wherein S may be oxidized and/or wherein the carbocycle or heterocycle may be unsubstituted or may be partially or fully substituted by substituents which, independently of each other, are selected from C₁-C₄-alkyl, C₁-C₄-haloalkyl, C₂-C₄-haloalkenyl, and ═O; and wherein R^{1b} is preferably H, C₁-C₄-alkyl, or OH;

and wherein

R^{d} and R^{e} are independently selected from NH₂ and C(═O)OH;

R^{f} is C₁-C₄-alkyl;

and wherein

R^{g} is H;

and wherein

R^{h} is halogen or C₁-C₄-alkoxy;

and wherein

Rⁱ is
(i) C₁-C₄-alkyl, C₁-C₄-haloalkyl, C₂-C₄-haloalkenyl; or
(iii) ═O.

The above defined compounds cover (a) compounds of formula I.3.A.X, in particular compounds of formula I.3.A.X.(R^{A})₁, I.3.A.X.(R^{A})₂, and I.3.A.X.(R^{A})₃, wherein R^{A} is defined according to option (i) in connection with the preferred substituent definitions defined above, (b) compounds of formula I.3.A.X, in particular compounds of formula I.3.A.X.(R^{A})₁, I.3.A.X.(R^{A})₂, and I.3.A.X.(R^{A})₃, wherein R^{A} is defined according to option (ii) in connection with the preferred substituent definitions defined above, (c) compounds of formula I.3.A.X, in particular compounds of formula I.3.A.X.(R^{A})₁, I.3.A.X.(R^{A})₂, and I.3.A.X.(R^{A})₃, wherein R^{A} is defined according to option (iii) in connection with the preferred substituent definitions defined above, and (d) compounds of formula I.3.A.X, in particular compounds of formula I.3.A.X.(R^{A})₁, I.3.A.X.(R^{A})₂, and I.3.A.X.(R^{A})₃, wherein R^{A} is defined according to option (iv) in connection with the preferred substituent definitions defined above. Compounds of formula I.3.A.X, in particular compounds of formula I.3.A.X.(R^{A})₁, I.3.A.X.(R^{A})₂, and I.3.A.X.(R^{A})₃, wherein R^{A} is defined according to option (i) in connection with the preferred substituent definitions defined above are especially preferred.

Especially preferred compounds of formula I are compounds of formula I.3.A.X, in particular compounds of formula I.3.A.X.(R^{A})₁, I.3.A.X.(R^{A})₂, or I.3.A.X.(R^{A})₃, wherein the substituent(s) R^{A} are independently of each other selected from halogen, CN, NH₂, C(═O)NR^{a}R^{b}, NHC(═O)NR^{a}R^{b}, NHC(═S)NR^{a}R^{b}, NHC(═O)H, C₁-C₄-alkoxy, C₂-C₄-alkynyl-C₁-C₂-hydroxyalkyl, and C₂-C₄-alkynyloxy, wherein R^{a} and R^{b} are in each case independently selected from H, C₁-C₂-alkyl, NH₂, C₁-C₂-hydroxyalkyl, or wherein R^{a} and R^{b} may together with the nitrogen atom to which they are bonded form a morpholine ring.

It has been shown by the inventors that such compounds are particularly suitable for reducing nitrification. Furthermore, the compounds are typically solid and have a low volatility.

Preferred compounds of formula I of the present invention are listed in the following Table 1. Said compounds are suitable for reducing nitrification and are typically non-volatile. Thus, the present invention preferably relates to any one of the novel compounds listed in Table 1.

In one aspect, the present invention relates to the use of any one of the compounds listed in Table 1 for reducing nitrification.

In another aspect, the present invention relates to an agricultural mixture comprising (i) a fertilizer and (ii) any one of the compounds listed in table 1 as a nitrification inhibitor.

In another aspect, the present invention relates to a composition comprising any one of the compounds listed in table 1 as a nitrification inhibitor and at least one carrier.

In yet another aspect, the present invention relates to a method for reducing nitrification as defined herein comprising the application of any one of the compounds listed in table 1.

Thus, compounds of formula I, which are selected from the compounds of the following table 1, are preferred in connection with the compositions, mixtures, uses and methods of the present invention. Particularly preferred according to the invention is the use of a nitrification inhibitor for reducing nitrification, wherein the nitrification inhibitor is a compound selected from the compounds of the following table 1.

TABLE 1

| Entry | |
|---|---|
| No. | |
| comp. A | Compound structure |
| 1 | 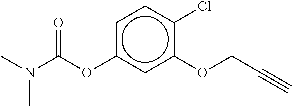 |
| 2 | 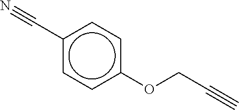 |
| 3 | 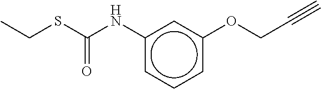 |
| 4 | 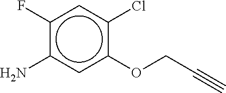 |
| 5 | 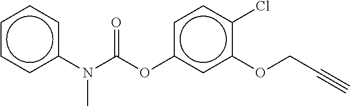 |
| 6 | 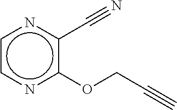 |
| 7 | 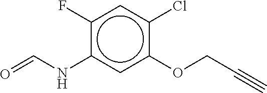 |
| 8 | 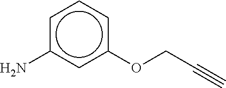 |
| 9 | 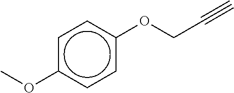 |
| 10 | 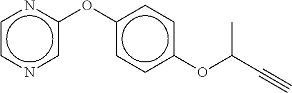 |
| 11 | 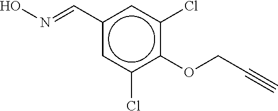 |
| 12 | 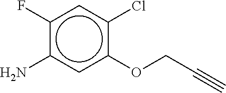 |
| 13 | 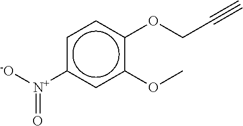 |
| 14 | 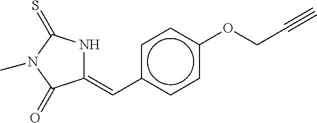 |
| 15 | 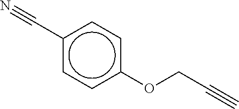 |
| 16 | 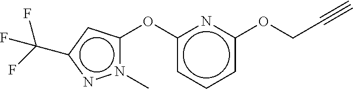 |
| 17 | 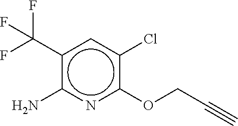 |
| 18 | 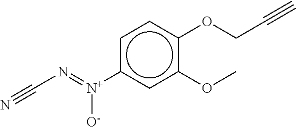 |
| 19 | 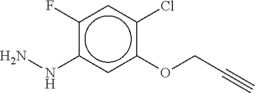 |
| 20 | 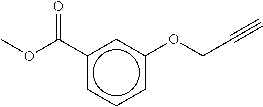 |
| 21 | 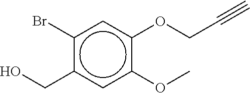 |
| 22 | 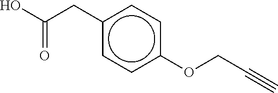 |
| 23 | 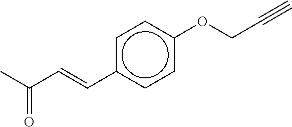 |
| 24 | 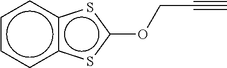 |
| 25 | 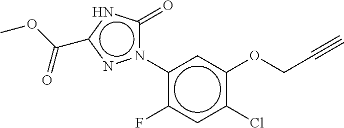 |
| 26 | 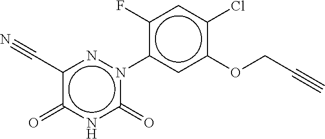 |
| 27 | 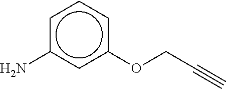 |
| 28 | 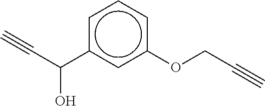 |
| 29 | 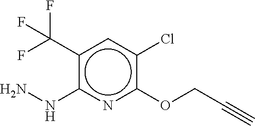 |
| 30 | 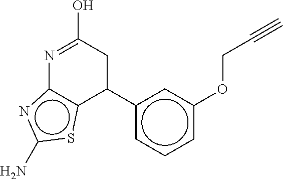 |
| 31 | 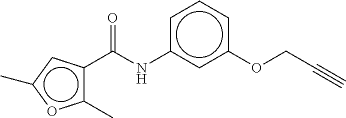 |
| 32 | 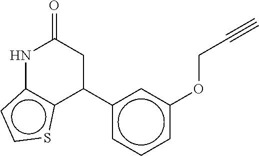 |
| 33 | 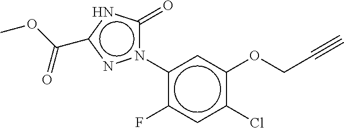 |
| 34 | 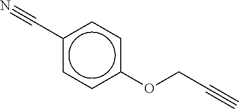 |
| 35 | 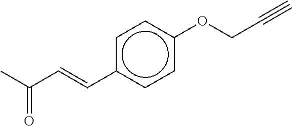 |
| 36 | 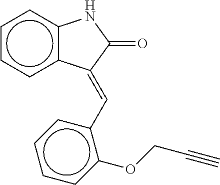 |
| 37 | 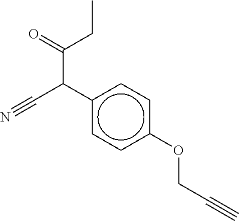 |
| 38 | 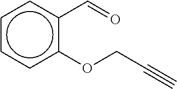 |
| 39 | 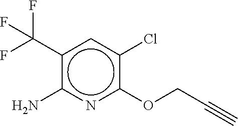 |
| 40 | 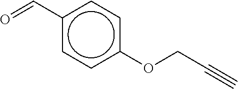 |
| 41 | 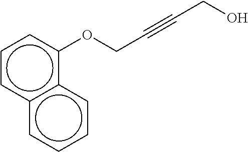 |
| 42 | 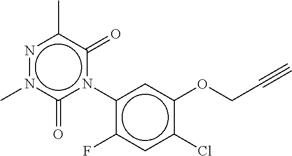 |
| 43 | 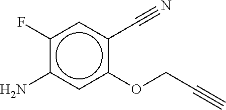 |
| 44 | 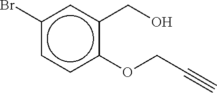 |
| 45 | 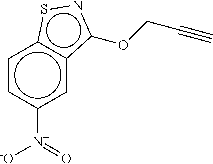 |
| 46 | 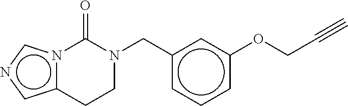 |
| 47 | 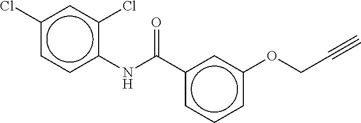 |
| 48 | 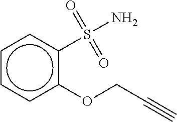 |
| 49 | 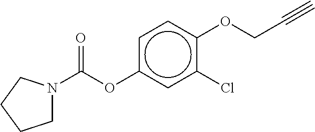 |
| 50 | 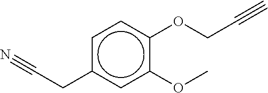 |
| 51 | 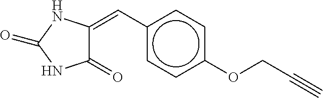 |
| 52 | 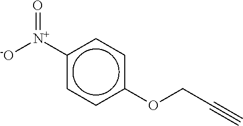 |
| 53 | 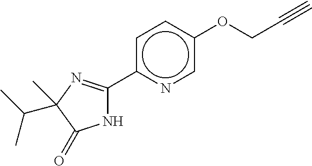 |
| 54 | 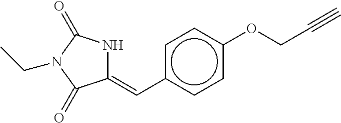 |
| 55 | 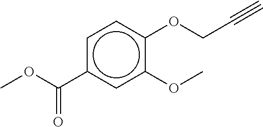 |
| 56 | 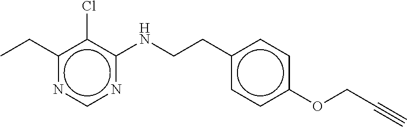 |
| 57 | 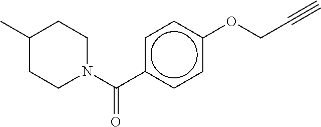 |
| 58 | 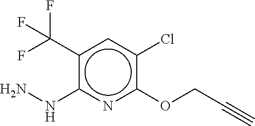 |
| 59 | 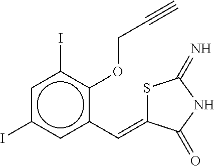 |
| 60 | 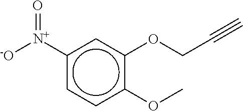 |
| 61 | 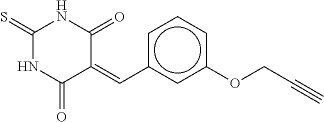 |
| 62 | 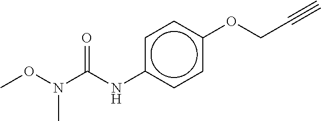 |
| 63 | 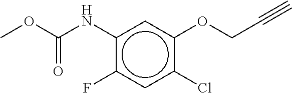 |
| 64 | 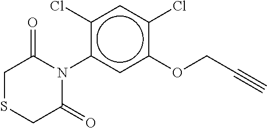 |
| 65 | 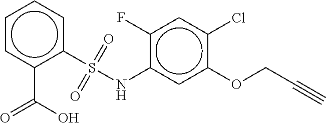 |
| 66 | 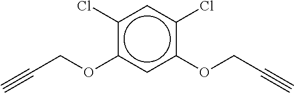 |
| 67 | 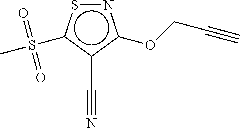 |
| 68 | 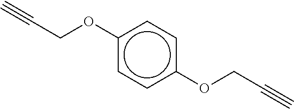 |
| 69 | 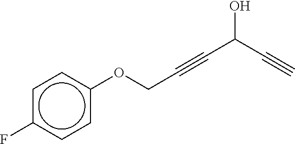 |
| 70 | 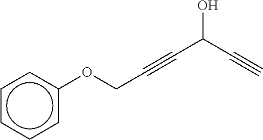 |
| 71 | 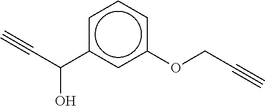 |
| 72 | 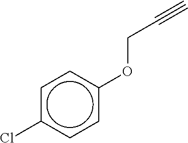 |
| 73 | 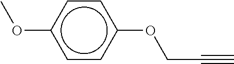 |
| 74 | 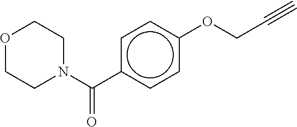 |
| 75 | 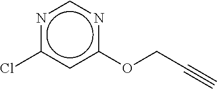 |
| 76 | 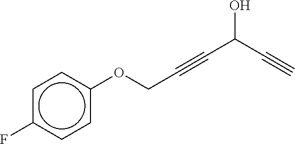 |
| 77 | 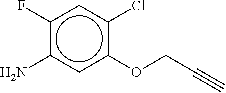 |
| 78 | 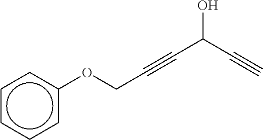 |
| 79 | 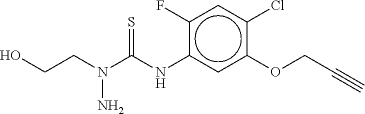 |
| 80 | 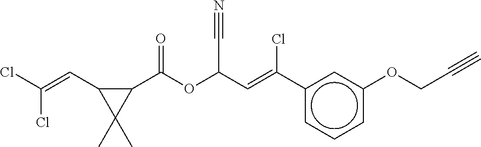 |
| 81 | 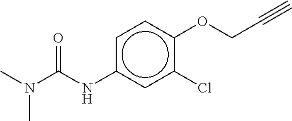 |
| 82 | 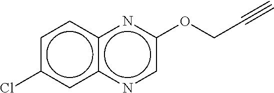 |
| 83 | 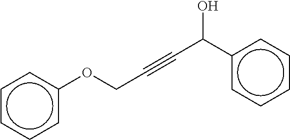 |
| 84 | 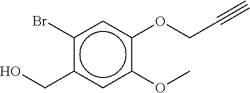 |
| 85 | 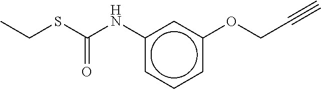 |
| 86 | 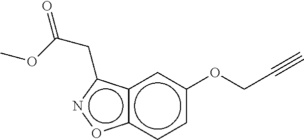 |
| 87 | 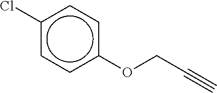 |
| 88 | 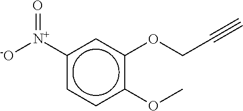 |
| 89 | 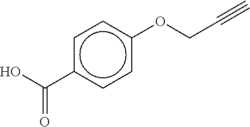 |
| 90 | 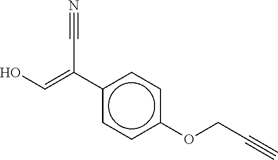 |
| 91 | 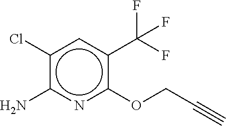 |
| 92 | 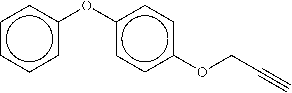 |
| 93 | 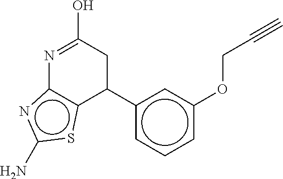 |
| 94 | 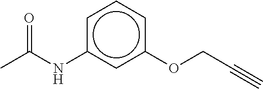 |
| 95 | 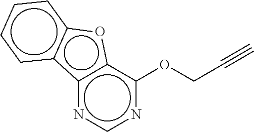 |
| 96 | 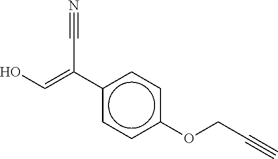 |
| 97 | 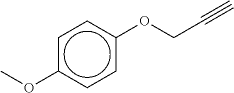 |
| 98 | 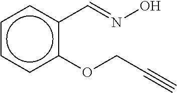 |
| 99 | 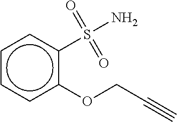 |
| 100 | 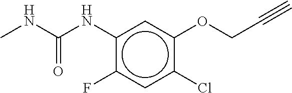 |
| 101 | 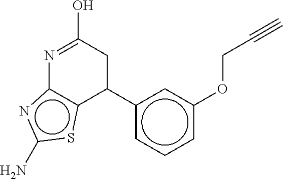 |
| 102 | 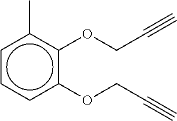 |
| 103 | 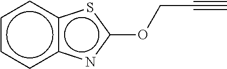 |
| 104 | 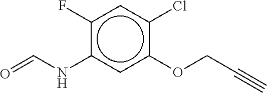 |
| 105 | 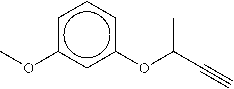 |
| 106 | 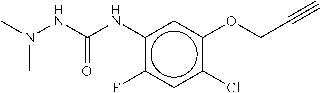 |
| 107 | 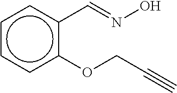 |
| 108 | 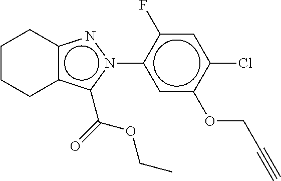 |
| 109 | 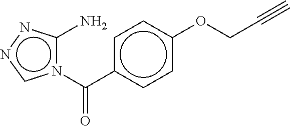 |
| 110 | 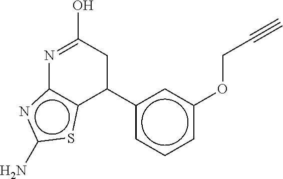 |
| 111 | 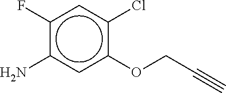 |
| 112 | 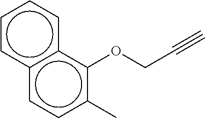 |
| 113 | 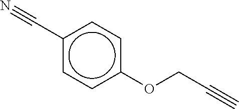 |
| 114 | 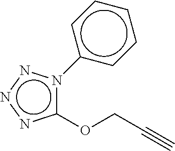 |
| 115 | 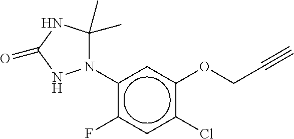 |
| 116 | 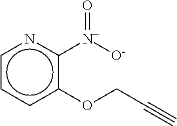 |
| 117 | 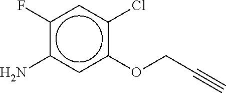 |
| 118 | 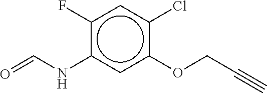 |
| 119 | 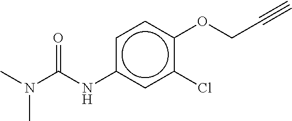 |
| 120 | 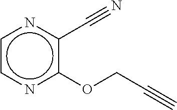 |
| 121 | 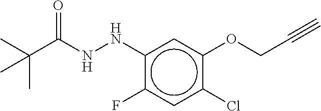 |
| 122 | 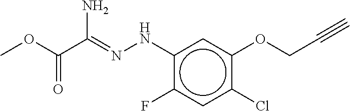 |
| 123 | 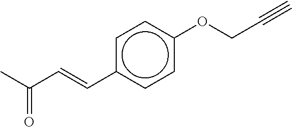 |
| 124 | 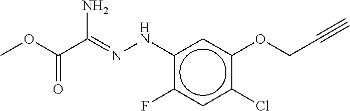 |
| 125 | 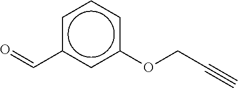 |
| 126 | 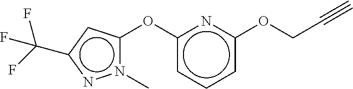 |
| 127 | 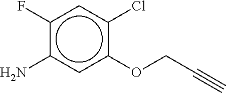 |
| 128 | 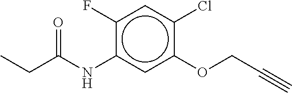 |
| 129 | 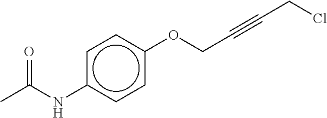 |
| 130 | 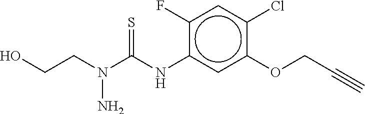 |
| 131 | 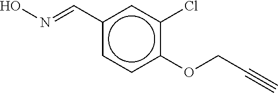 |
| 132 | 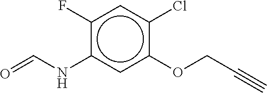 |
| 133 | 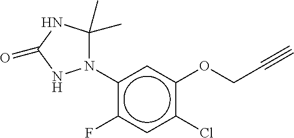 |
| 134 | 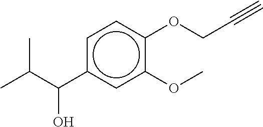 |
| 135 | 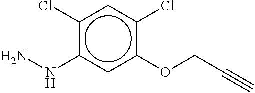 |
| 136 | 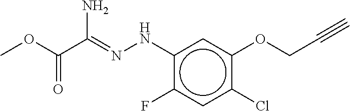 |
| 137 | 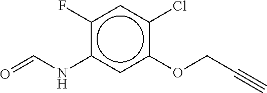 |
| 138 | 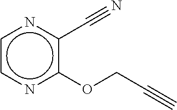 |
| 139 | 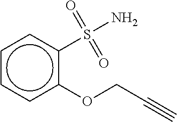 |
| 140 | 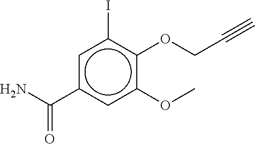 |
| 141 | 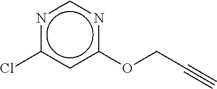 |
| 142 | 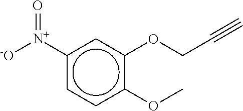 |
| 143 | 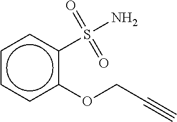 |
| 144 | 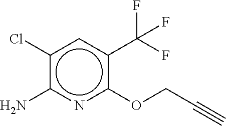 |
| 145 | 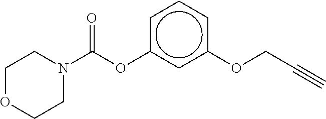 |
| 146 | 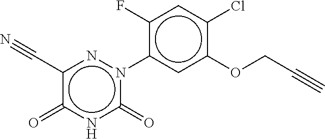 |
| 147 | 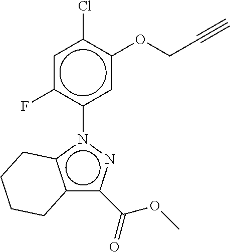 |
| 148 | 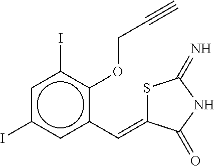 |
| 149 | 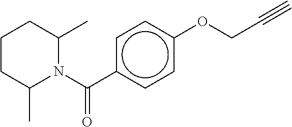 |
| 150 | 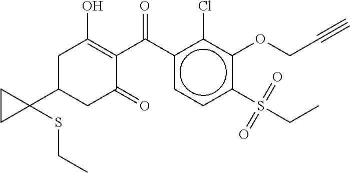 |
| 151 | 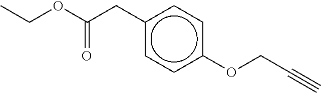 |
| 152 | 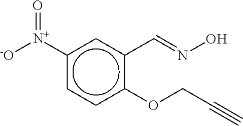 |
| 153 | 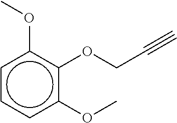 |
| 154 | 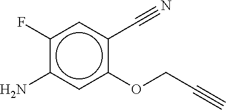 |
| 155 | 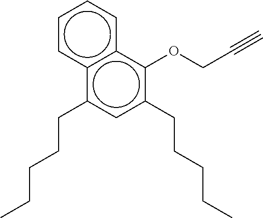 |
| 156 | 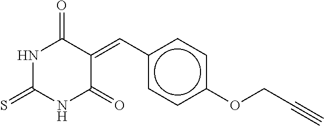 |
| 157 | 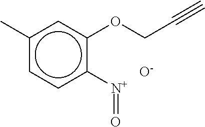 |
| 158 | 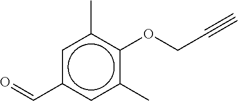 |
| 159 | 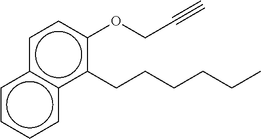 |
| 160 | 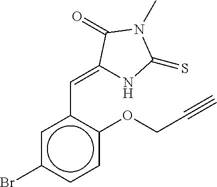 |
| 161 | 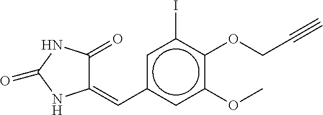 |
| 162 | 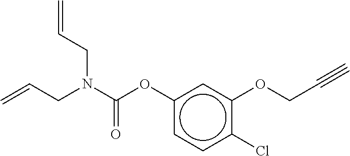 |
| 163 | 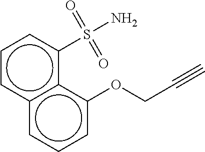 |
| 164 | 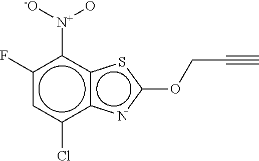 |
| 165 | 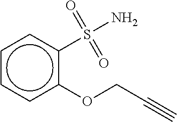 |
| 166 | 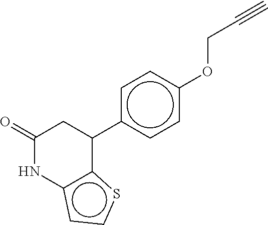 |
| 167 | 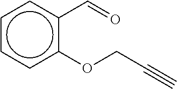 |
| 168 | 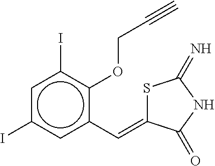 |
| 169 | 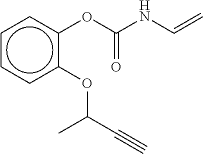 |
| 170 | 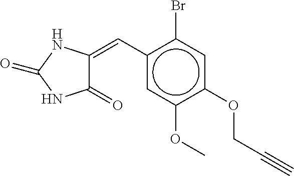 |
| 171 | 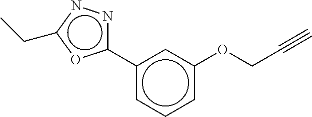 |
| 172 | 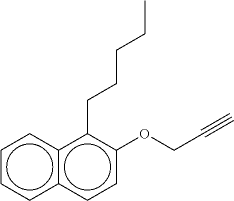 |
| 173 | 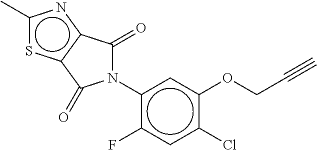 |
| 174 | 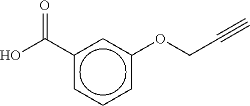 |
| 175 | 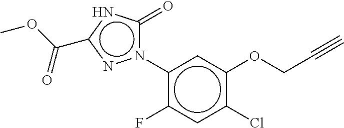 |
| 176 | 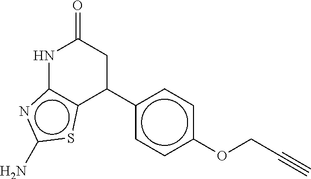 |
| 177 | 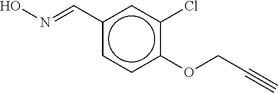 |
| 178 | 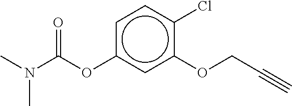 |
| 179 | 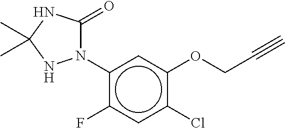 |
| 180 | 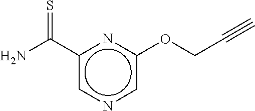 |
| 181 | 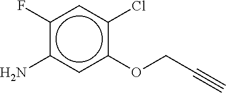 |
| 182 | 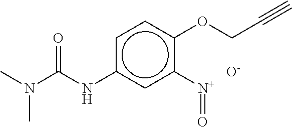 |
| 183 | 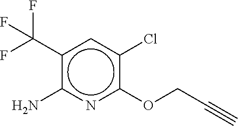 |
| 184 | 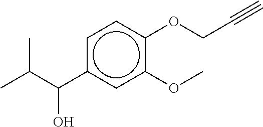 |
| 185 | 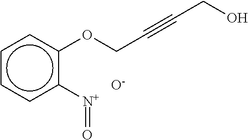 |
| 186 | 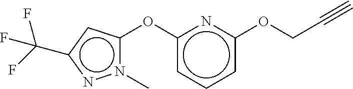 |
| 187 | 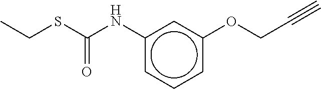 |
| 188 | 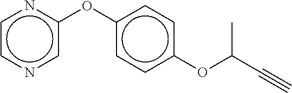 |
| 189 | 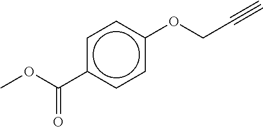 |
| 190 | 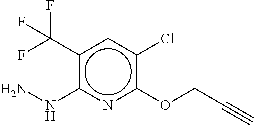 |
| 191 | 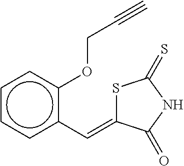 |
| 192 | 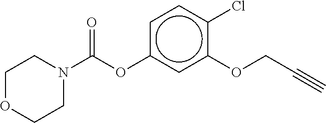 |
| 193 | 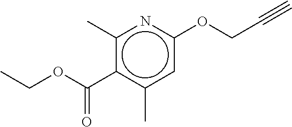 |
| 194 | 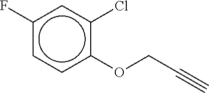 |
| 195 | 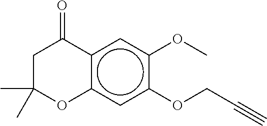 |
| 196 | 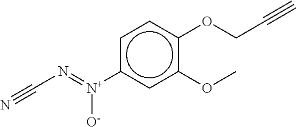 |
| 197 | 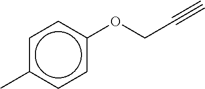 |
| 198 | 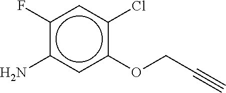 |
| 199 | 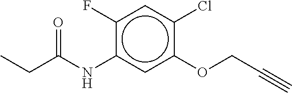 |
| 200 | 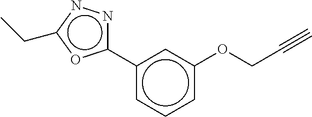 |
| 201 | 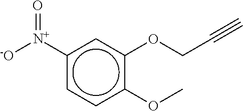 |
| 202 | 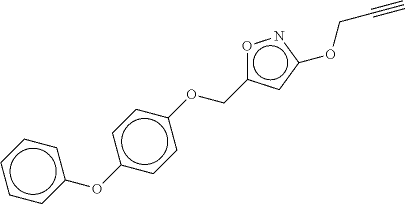 |
| 203 | 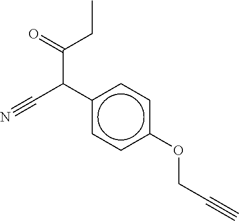 |
| 204 | 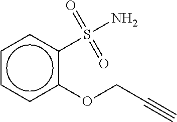 |
| 205 | 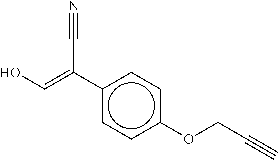 |
| 206 | 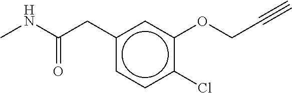 |
| 207 | 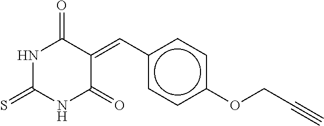 |
| 208 | 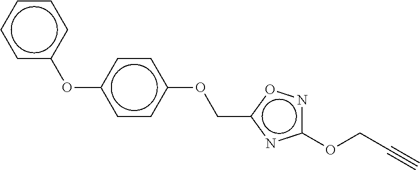 |
| 209 | 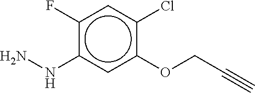 |
| 210 | 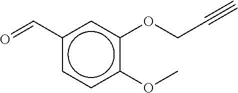 |
| 211 | 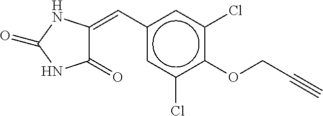 |
| 212 | 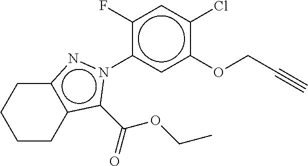 |
| 213 | 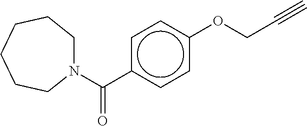 |
| 214 | 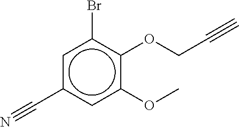 |
| 215 | 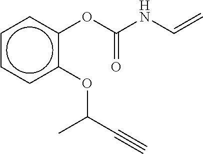 |

In a central aspect the present invention thus relates to the use of a nitrification inhibitor as defined herein above, or of a composition comprising said nitrification inhibitor as defined herein for reducing nitrification. The nitrification inhibitor or derivatives or salts thereof as defined herein above, in particular compounds of formula I and/or salts or suitable derivatives thereof, as well as compositions comprising said nitrification inhibitor, or agrochemical mixtures comprising said nitrification inhibitor as defined herein may be used for reducing nitrification.

In a central aspect the present invention thus relates to the use of a compound of formula I as defined herein, in particular any one of the compounds listed in Table 1 above, for reducing nitrification, or to the use of a composition comprising any one of the compounds listed in Table 1 and a carrier for reducing nitrification. Furthermore, the present invention releates to an agricultural mixture comprising any one of the compounds listed in Table 1 above and at least one fertilizer as defined herein. The compounds of formula I or derivatives or salts thereof as defined herein, in particular compounds of formula I and/or salts thereof, as well as compositions comprising said compound of formula I, or agrochemical mixtures comprising said compound of formula I may be used for reducing nitrification.

It has surprisingly been found that the phenylpropargylether compounds 68, 71, 72, 73, 74, 77, 79, 81, 113, and 118 of Table 1 above provide a good balance in terms of the effectiveness as nitrification inhibitors, stability/low volatility, and environmental safety.

Thus, the compounds of formula I are preferably selected from the compounds 68, 71, 72, 73, 74, 77, 79, 81, 113, and 118, and these compounds are also preferred in connection with the uses, methods, compositions and mixtures of the invention.

In a preferred embodiment, the compound of formula I as defined herein in connection with the uses, methods, compositions and mixtures of the invention is not oxadiargyl, i.e. 5-tert-butyl-3-[2,4-dichloro-5-(prop-2-ynyloxy)phenyl]-1,3,4-oxadiazol-2(3H)-one.

The use according to the present invention may be based on an application of the nitrification inhibitor, the composition or the agrochemical mixture as defined herein to a plant growing on soil and/or the locus where the plant is growing or is intended to grow, or the use may be based on the application of the nitrification inhibitor, the composition or the agrochemical mixture as defined herein to soil where a plant is growing or is intended to grow or to soil substituents. In specific embodiments, the nitrification inhibitor may be used for reducing nitrification in the absence of plants, e.g. as preparatory activity for subsequent agricultural activity, or for reducing nitrification in other technical areas, which are not related to agriculture, e.g. for environmental, water protection, energy production or similar purposes. In specific embodiments, the nitrification inhibitor, or a composition comprising said nitrification inhibitor according to the present invention may be used for the reduction of nitrification in sewage, slurry, manure or dung of animals, e.g. swine or bovine feces. For example, the nitrification inhibitor, or a composition comprising said nitrification inhibitor according to the present invention may be used for the reduction of nitrification in sewage plants, biogas plants, cowsheds, liquid manure tanks or containers etc. In further embodiments, the nitrification inhibitor, or a composition comprising said nitrification inhibitor according to the present invention may be used for the reduction of nitrification in situ in animals, e.g. in productive livestock. Accordingly, the nitrification inhibitor, or a composition comprising said nitrification inhibitor according to the present invention may be fed to an animal, e.g. a mammal, for instance together with suitable feed and thereby lead to a reduction of nitrification in the gastrointestinal tract of the animals, which in turn is resulting in reduction of emissions from the gastrointestinal tract. This activity, i.e. the feeding of nitrification inhibitor, or a composition comprising said nitrification inhibitor according to the present invention may be repeated one to several times, e.g. each 2^{nd}, 3^{rd}, 4^{th}, 5^{th}, 6^{th}, 7^{th} day, or each week, 2 weeks, 3 weeks, or month, 2 months etc.

The use may further include the application of a nitrification inhibitor or derivatives or salts thereof as defined herein above, in particular compounds of formula I and/or salts or suitable derivatives thereof, as well as compositions comprising said nitrification inhibitor, or agrochemical mixtures comprising said nitrification inhibitor as defined herein above to environments, areas or zones, where nitrification takes place or is assumed or expected to take place. Such environments, areas or zones may not comprise plants or soil. For example, the inhibitors may be used for nitrification inhibition in laboratory environments, e.g. based on enzymatic reactions or the like. Also envisaged is the use in green houses or similar indoor facilities.

The term “reducing nitrification” or “reduction of nitrification” as used herein refers to a slowing down or stopping of nitrification processes, e.g. by retarding or eliminating the natural transformation of ammonium into nitrate. Such reduction may be a complete or partial elimination of nitrification at the plant or locus where the inhibitor or composition comprising said inhibitor is applied. For example, a partial elimination may result in a residual nitrification on or in the plant, or in or on the soil or soil substituents where a plant grows or is intended to grow of about 90% to 1%, e.g. 90%, 85%, 80%, 70%, 60%, 50%, 40%, 30%, 20%, 10% or less than 10%, e.g. 5% or less than 5% in comparison to a control situation where the nitrification inhibitor is not used. In certain embodiments, a partial elimination may result in a residual nitrification on or in the plant or in or on the soil or soil substituents where a plant grows or is intended to grow of below 1%, e.g. at 0.5%, 0.1% or less in comparison to a control situation where the nitrification inhibitor is not used.

The use of a nitrification inhibitor as defined herein above, or of a composition as defined herein for reducing nitrification may be a single use, or it may be a repeated use. As single use, the nitrification inhibitor or corresponding compositions may be provided to their target sites, e.g. soil or loci, or objects, e.g. plants, only once in a physiologically relevant time interval, e.g. once a year, or once every 2 to 5 years, or once during the lifetime of a plant.

In other embodiments, the use may be repeated at least once per time period, e.g. the nitrification inhibitor as defined herein above, or a composition as defined herein may be used for reducing nitrification at their target sites or objects two times within a time interval of days, weeks or months. The term “at least once” as used in the context of a use of the nitrification inhibitor means that the inhibitor may be used two times, or several times, i.e. that a repetition or multiple repetitions of an application or treatment with a nitrification inhibitor may be envisaged. Such a repetition may be a 2 times, 3 times, 4 times, 5 times, 6 times, 7 times, 8 times, 9 times, 10 times or more frequent repetition of the use.

The nitrification inhibitor according to the present invention may be used in any suitable form. For example, it may be used as coated or uncoated granule, in liquid or semi-liquid form, as sprayable entity, or in irrigation approaches etc. In specific embodiments, the nitrification inhibitor as defined herein may be applied or used as such, i.e. without formulations, fertilizer, additional water, coatings, or any further ingredient.

The term “irrigation” as used herein refers to the watering of plants or loci or soils or soil substituents where a plant grows or is intended to grow, wherein said watering includes the provision of the nitrification inhibitor according to the present invention together with water.

In a further aspect the invention relates to a composition for reducing nitrification comprising at least one nitrification inhibitor wherein said nitrification inhibitor is a compound of formula I or a derivative as defined herein above; and at least one carrier.

The term “composition for reducing nitrification” as used herein refers to a composition which is suitable, e.g. comprises effective concentrations and amounts of ingredients such as nitrification inhibitors, in particular compounds of formula I or derivatives as defined herein, for reducing nitrification in any context or environment in which nitrification may occur. In one embodiment, the nitrification may be reduced in or on or at the locus of a plant. Typically, the nitrification may be reduced in the root zone of a plant. However, the area in which such reduction of nitrification may occur is not limited to the plants and their environment, but may also include any other habitat of nitrifying bacteria or any site at which nitrifying enzymatic activities can be found or can function in a general manner, e.g. sewage plants, biogas plants, animal effluents from productive livestock, e.g. cows, pigs etc. “Effective amounts” or “effective concentrations” of nitrification inhibitors as defined herein may be determined according to suitable in vitro and in vivo testings known to the skilled person. These amounts and concentrations may be adjusted to the locus, plant, soil, climate conditions or any other suitable parameter which may have an influence on nitrification processes.

A “carrier” as used herein is a substance or composition which facilitates the delivery and/or release of the ingredients to the place or locus of destination. The term includes, for instance, agrochemical carriers which facilitate the delivery and/or release of agrochemicals in their field of use, in particular on or into plants.

Examples of suitable carriers include solid carriers such as phytogels, or hydrogels, or mineral earths e.g. silicates, silica gels, talc, kaolins, limestone, lime, chalk, bole, loess, clays, dolomite, diatomaceous earth, calcium sulfate, magnesium sulfate, magnesium oxide, ground synthetic materials, fertilizers, such as, e.g., an solid or liquid ammonium-containing inorganic fertilizer such as an NPK fertilizer, ammonium nitrate, calcium ammonium nitrate, ammonium sulfate nitrate, ammonium sulfate or ammonium phosphate; an solid or liquid organic fertilizer such as liquid manure, semi-liquid manure, stable manure, biogas manure and straw manure, worm castings, compost, seaweed or guano, or an urea-containing fertilizer such as urea, formaldehyde urea, urea ammonium nitrate (UAN) solution, urea sulphur, stabilized urea, urea based NPK-fertilizers, or urea ammonium sulfate, in particular urea, urea based NPK, UAN, ammonium sulfate, ammonium phosphate, ammonium nitrate, or urea, and products of vegetable origin, such as cereal meal, tree bark meal, wood meal and nutshell meal, cellulose powders and other solid carriers. Further suitable examples of carriers include fumed silica or precipitated silica, which may, for instance, be used in solid formulations as flow aid, anti-caking aid, milling aid and as carrier for liquid active ingredients. Additional examples of suitable carriers are microparticles, for instance microparticles which stick to plant leaves and release their content over a certain period of time. In specific embodiments, agrochemical carriers such as composite gel microparticles that can be used to deliver plant-protection active principles, e.g. as described in U.S. Pat. No. 6,180,141; or compositions comprising at least one phytoactive compound and an encapsulating adjuvant, wherein the adjuvant comprises a fungal cell or a fragment thereof, e.g. as described in WO 2005/102045; or carrier granules, coated with a lipophilic tackifier on the surface, wherein the carrier granule adheres to the surface of plants, grasses and weeds, e.g. as disclosed in US 2007/0280981 may be used. In further specific embodiments, such carriers may include specific, strongly binding molecule which assure that the carrier sticks to the plant, the seed, and/or loci where the plant is growing or is intended to grow, till its content is completely delivered. For instance, the carrier may be or comprise cellulose binding domains (CBDs) have been described as useful agents for attachment of molecular species to cellulose (see U.S. Pat. No. 6,124,117); or direct fusions between a CBD and an enzyme; or a multifunctional fusion protein which may be used for delivery of encapsulated agents, wherein the multifunctional fusion proteins may consist of a first binding domain which is a carbohydrate binding domain and a second binding domain, wherein either the first binding domain or the second binding domain can bind to a microparticle (see also WO 03/031477). Further suitable examples of carriers include bifunctional fusion proteins consisting of a CBD and an anti-RR6 antibody fragment binding to a microparticle, which complex may be deposited onto treads or cut grass (see also WO 03/031477). In another specific embodiment the carrier may be active ingredient carrier granules that adhere to the surface of plants, grasses and weeds, the seed, and/or loci where the plant is growing or is intended to grow, etc. using a moisture-active coating, for instance including gum arabic, guar gum, gum karaya, gum tragacanth and locust bean gum. Upon application of the inventive granule onto a plant surface, water from precipitation, irrigation, dew, co-application with the granules from special application equipment, or guttation water from the plant itself may provide sufficient moisture for adherence of the granule to the plant surface (see also US 2007/0280981).

In another specific embodiment the carrier, e.g. an agrochemical carrier, may be or comprise polyaminoacids. Polyaminoacids may be obtained according to any suitable process, e.g. by polymerization of single or multiple amino acids such as glycine, alanine, valine, leucine, isoleucine, phenylalanine, proline, tryptophan, serine, tyrosine, cysteine, methionine, asparagine, glutamine, threonine, aspartic acid, glutamic acid, lysine, arginine, histidine and/or ornithine. Polyaminoacids may be combined with a nitrification inhibitor according to the present invention and, in certain embodiments, also with further carriers as mentioned herein above, or other nitrification inhibitors as mentioned herein in any suitable ratio. For example, Polyaminoacids may be combined with a nitrification inhibitor according to the present invention in a ratio of 1 to 10 (polyaminoacids) vs. 0.5 to 2 (nitrification inhibitor according to the present invention).

The composition for reducing nitrification comprising at least one nitrification inhibitor as defined herein may further comprise additional ingredients, for example at least one pesticidal compound. For example, the composition may additionally comprise at least one herbicidal compound and/or at least one fungicidal compound and/or at least one insecticidal compound and/or at least one nematicide and/or at least one biopesticide and/or at least one biostimulant.

In further embodiments, the composition may, in addition to the above indicated ingredients, in particular in addition to the nitrification inhibitor of the compound of formula I, further comprise one or more alternative or additional nitrification inhibitors. Examples of envisaged alternative or additional nitrification inhibitors are linoleic acid, alpha-linolenic acid, methyl p-coumarate, methyl ferulate, methyl 3-(4-hydroxyphenyl) propionate (MHPP), Karanjin, brachialacton, p-benzoquinone sorgoleone, 2-chloro-6-(trichloromethyl)-pyridine (nitrapyrin or N-serve), dicyandiamide (DCD, DIDIN), 3,4-dimethyl pyrazole phosphate (DMPP, ENTEC), 4-amino-1,2,4-triazole hydrochloride (ATC), 1-amido-2-thiourea (ASU), 2-amino-4-chloro-6-methylpyrimidine (AM), 2-mercapto-benzothiazole (MBT), 5-ethoxy-3-trichloromethyl-1,2,4-thiodiazole (terrazole, etridiazole), 2-sulfanilamidothiazole (ST), ammoniumthiosulfate (ATU), 3-methylpyrazol (3-MP), 3,5-dimethylpyrazole (DMP), 1,2,4-triazol and thiourea (TU), N-(1H-pyrazolyl-methyl)acetamides such as N-((3(5)-methyl-1H-pyrazole-1-yl)methyl)acetamide, and N-(1H-pyrazolyl-methyl)formamides such as N-((3(5)-methyl-1H-pyrazole-1-yl)methyl formamide, N-(4-chloro-3(5)-methyl-pyrazole-1-ylmethyl)-formamide, N-(3(5),4-dimethyl-pyrazole-1-ylmethyl)-formamide, neem, products based on ingredients of neem, cyan amide, melamine, zeolite powder, catechol, benzoquinone, sodium terta board, or zinc sulfate.

In a preferred embodiment, the composition according to the present invention may comprise a combination of the nitrification inhibitor of the compound of formula I and 2-chloro-6-(trichloromethyl)-pyridine (nitrapyrin or N-serve).

In a further preferred embodiment, the composition according to the present invention may comprise a combination of the nitrification inhibitor of the compound of formula I and 5-ethoxy-3-trichloromethyl-1,2,4-thiodiazole (terrazole, etridiazole).

In a further preferred embodiment, the composition according to the present invention may comprise a combination of the nitrification inhibitor of the compound of formula I and dicyandiamide (DCD, DIDIN).

In a further preferred embodiment, the composition according to the present invention may comprise a combination of the nitrification inhibitor of the compound of formula I and 3,4-dimethyl pyrazole phosphate (DMPP, ENTEC).

In a further preferred embodiment, the composition according to the present invention may comprise a combination of the nitrification inhibitor of the compound of formula I and 2-amino-4-chloro-6-methylpyrimidine (AM).

In a further preferred embodiment, the composition according to the present invention may comprise a combination of the nitrification inhibitor of the compound of formula I and 2-mercapto-benzothiazole (MBT).

In a further preferred embodiment, the composition according to the present invention may comprise a combination of the nitrification inhibitor of the compound of formula I and 2-sulfanilamidothiazole (ST).

In a further preferred embodiment, the composition according to the present invention may comprise a combination of the nitrification inhibitor of the compound of formula I and ammoniumthiosulfate (ATU).

In a further preferred embodiment, the composition according to the present invention may comprise a combination of the nitrification inhibitor of the compound of formula I and 3-methylpyrazol (3-MP).

In a further preferred embodiment, the composition according to the present invention may comprise a combination of the nitrification inhibitor of the compound of formula I and 3,5-dimethylpyrazole (DMP).

In a further preferred embodiment, the composition according to the present invention may comprise a combination of the nitrification inhibitor of the compound of formula I and 1,2,4-triazol.

In a further preferred embodiment, the composition according to the present invention may comprise a combination of the nitrification inhibitor of the compound of formula I and thiourea (TU).

In yet another preferred embodiment, the composition according to the present invention may comprise a combination of the nitrification inhibitor of the compound of formula I and linoleic acid.

In yet another preferred embodiment, the composition according to the present invention may comprise a combination of the nitrification inhibitor of the compound of formula I and alpha-linolenic acid.

In yet another preferred embodiment, the composition according to the present invention may comprise a combination of the nitrification inhibitor of the compound of formula I and methyl p-coumarate.

In yet another preferred embodiment, the composition according to the present invention may comprise a combination of the nitrification inhibitor of the compound of formula I and methyl 3-(4-hydroxyphenyl) propionate (MHPP).

In yet another preferred embodiment, the composition according to the present invention may comprise a combination of the nitrification inhibitor of the compound of formula I and methyl ferulate.

In yet another preferred embodiment, the composition according to the present invention may comprise a combination of the nitrification inhibitor of the compound of formula I and Karanjin.

In yet another preferred embodiment, the composition according to the present invention may comprise a combination of the nitrification inhibitor of the compound of formula I and brachialacton.

In yet another preferred embodiment, the composition according to the present invention may comprise a combination of the nitrification inhibitor of the compound of formula I and p-benzoquinone sorgoleone.

In yet another preferred embodiment, the composition according to the present invention may comprise a combination of the nitrification inhibitor of the compound of formula I and 4-amino-1,2,4-triazole hydrochloride (ATC).

In yet another preferred embodiment, the composition according to the present invention may comprise a combination of the nitrification inhibitor of the compound of formula I and 1-amido-2-thiourea (ASU).

In yet another preferred embodiment, the composition according to the present invention may comprise a combination of the nitrification inhibitor of the compound of formula I and N-((3(5)-methyl-1 H-pyrazole-1-yl)methyl)acetamide.

In yet another preferred embodiment, the composition according to the present invention may comprise a combination of the nitrification inhibitor of the compound of formula I and N-((3(5)-methyl-1 H-pyrazole-1-yl)methyl formamide.

In yet another preferred embodiment, the composition according to the present invention may comprise a combination of the nitrification inhibitor of the compound of formula I and N-(4-chloro-3(5)-methyl-pyrazole-1-ylmethyl)-formamide.

In yet another preferred embodiment, the composition according to the present invention may comprise a combination of the nitrification inhibitor of the compound of formula I and N-(3(5),4-dimethyl-pyrazole-1-ylmethyl)-formamide.

In yet another preferred embodiment, the composition according to the present invention may comprise a combination of the nitrification inhibitor of the compound of formula I and neem, or products based on neem.

In yet another preferred embodiment, the composition according to the present invention may comprise a combination of the nitrification inhibitor of the compound of formula I and cyanamide.

In yet another preferred embodiment, the composition according to the present invention may comprise a combination of the nitrification inhibitor of the compound of formula I and melamine.

In yet another preferred embodiment, the composition according to the present invention may comprise a combination of the nitrification inhibitor of the compound of formula I and zeolite powder.

In yet another preferred embodiment, the composition according to the present invention may comprise a combination of the nitrification inhibitor of the compound of formula I and batechol.

In yet another preferred embodiment, the composition according to the present invention may comprise a combination of the nitrification inhibitor of the compound of formula I and benzoquinone.

In yet another preferred embodiment, the composition according to the present invention may comprise a combination of the nitrification inhibitor of the compound of formula I and sodium terat borate.

In yet another preferred embodiment, the composition according to the present invention may comprise a combination of the nitrification inhibitor of the compound of formula I and zinc sulfate.

In further embodiments, the composition according to the present invention may comprise a combination of the nitrification inhibitor of the compound of formula I and two entities selected from the group comprising: linoleic acid, alpha-linolenic acid, methyl p-coumarate, methyl ferulate, methyl 3-(4-hydroxyphenyl) propionate (MHPP), Karanjin, brachialacton, p-benzoquinone sorgoleone, 2-chloro-6-(trichloromethyl)-pyridine (nitrapyrin or N-serve), dicyandiamide (DCD, DIDIN), 3,4-dimethyl pyrazole phosphate (DMPP, ENTEC), 4-amino-1,2,4-triazole hydrochloride (ATC), 1-amido-2-thiourea (ASU), 2-amino-4-chloro-6-methylpyrimidine (AM), 2-mercapto-benzothiazole (MBT), 5-ethoxy-3-trichloromethyl-1,2,4-thiodiazole (terrazole, etridiazole), 2-sulfanilamidothiazole (ST), ammoniumthiosulfate (ATU), 3-methylpyrazol (3-MP), 3,5-dimethylpyrazole (DMP), 1,2,4-triazol and thiourea (TU), N-(1H-pyrazolyl-methyl)acetamides such as N-((3(5)-methyl-1H-pyrazole-1-yl)methyl)acetamide, and N-(1H-pyrazolyl-methyl)formamides such as N-((3(5)-methyl-1H-pyrazole-1-yl)methyl formamide, N-(4-chloro-3(5)-methyl-pyrazole-1-ylmethyl)-formamide, or N-(3(5),4-dimethyl-pyrazole-1-ylmethyl)-formamide, neem, products based on ingredients of neem, cyan amide, melamine, zeolite powder, catechol, benzoquinone, sodium terta board, zinc sulfate.

In yet another group of embodiments, the composition according to the present invention may comprise a combination of the nitrification inhibitor of the compound of formula I and three, four or more entities selected from the group comprising: linoleic acid, alpha-linolenic acid, methyl p-coumarate, methyl ferulate, methyl 3-(4-hydroxyphenyl) propionate (MHPP), Karanjin, brachialacton, p-benzoquinone sorgoleone, 2-chloro-6-(trichloromethyl)-pyridine (nitrapyrin or N-serve), dicyandiamide (DCD, DIDIN), 3,4-dimethyl pyrazole phosphate (DMPP, ENTEC), 4-amino-1,2,4-triazole hydrochloride (ATC), 1-amido-2-thiourea (ASU), 2-amino-4-chloro-6-methylpyrimidine (AM), 2-mercapto-benzothiazole (MBT), 5-ethoxy-3-trichloromethyl-1,2,4-thiodiazole (terrazole, etridiazole), 2-sulfanilamidothiazole (ST) ammoniumthiosulfate (ATU), 3-methylpyrazol (3-MP), 3,5-dimethylpyrazole (DMP), 1,2,4-triazol and thiourea (TU), N-(1H-pyrazolyl-methyl)acetamides such as N-((3(5)-methyl-1H-pyrazole-1-yl)methyl)acetamide, and N-(1H-pyrazolyl-methyl)formamides such as N-((3(5)-methyl-1H-pyrazole-1-yl)methyl formamide, N-(4-chloro-3(5)-methyl-pyrazole-1-ylmethyl)-formamide, or N-(3(5),4-dimethyl-pyrazole-1-ylmethyl)-formamide, neem, products based on ingredients of neem, cyan amide, melamine, zeolite powder, catechol, benzoquinone, sodium terta board, zinc sulfate.

In further embodiments, the composition may, in addition to the above indicated ingredients, in particular in addition to the nitrification inhibitor of the compound of formula I, further comprise one or more urease inhibitors. Examples of envisaged urease inhibitors include N-(n-butyl) thiophosphoric acid triamide (NBPT, Agrotain), N-(n-propyl) thiophosphoric acid triamide (NPPT), 2-nitrophenyl phosphoric triamide (2-NPT), further NXPTs known to the skilled person, phenylphosphorodiamidate (PPD/PPDA), hydroquinone, ammonium thiosulfate, and mixtures of NBPT and NPPT (see e.g. U.S. Pat. No. 8,075,659). Such mixtures of NBPT and NPPT may comprise NBPT in amounts of from 40 to 95% wt.-% and preferably of 60 to 80% wt.-% based on the total amount of active substances. Such mixtures are marketed as LIMUS, which is a composition comprising about 16.9 wt.-% N BPT and about 5.6 wt.-% N PPT and about 77.5 wt.-% of other ingredients including solvents and adjuvants.

In a preferred embodiment, the composition according to the present invention may comprise a combination of the nitrification inhibitor of the compound of formula I and N-(n-butyl) thiophosphoric acid triamide (NBPT, Agrotain).

In a further preferred embodiment, the composition according to the present invention may comprise a combination of the nitrification inhibitor of the compound of formula I and phenylphosphorodiamidate (PPD/PPDA).

In a further preferred embodiment, the composition according to the present invention may comprise a combination of the nitrification inhibitor of the compound of formula I and N-(n-propyl) thiophosphoric acid triamide (NPPT).

In a further preferred embodiment, the composition according to the present invention may comprise a combination of the nitrification inhibitor of the compound of formula I and 2-nitrophenyl phosphoric triamide (2-NPT).

In a further preferred embodiment, the composition according to the present invention may comprise a combination of the nitrification inhibitor of the compound of formula I and hydroquinone.

In a further preferred embodiment, the composition according to the present invention may comprise a combination of the nitrification inhibitor of the compound of formula I and ammonium thiosulfate.

In yet another preferred embodiment, the composition according to the present invention may comprise a combination of the nitrification inhibitor of the compound of formula I and neem.

In yet another preferred embodiment, the composition according to the present invention may comprise a combination of the nitrification inhibitor of the compound of formula I and cyanamide.

In yet another preferred embodiment, the composition according to the present invention may comprise a combination of the nitrification inhibitor of the compound of formula I and melamine.

In a further preferred embodiment, the composition according to the present invention may comprise a combination of the nitrification inhibitor of the compound of formula I and a mixture of NBPT and NPPT such as LIMUS.

In further embodiments, the composition according to the present invention may comprise a combination of the nitrification inhibitor of the compound of formula I and two or more entities selected from the group comprising: N-(n-butyl) thiophosphoric acid triamide (NBPT, Agrotain), N-(n-propyl) thiophosphoric acid triamide (NPPT), 2-nitrophenyl phosphoric triamide (2-NPT), further NXPTs known to the skilled person, phenylphosphorodiamidate (PPD/PPDA), hydroquinone, ammonium thiosulfate, and LIMUS.

In further embodiments, the composition may, in addition to one, more or all of the above indicated ingredients, in particular in addition to the nitrification inhibitor of the compound of formula I, further comprise one or more plant growth regulators. Examples of envisaged plant growth regulators are antiauxins, auxins, cytokinins, defoliants, ethylene modulators, ethylene releasers, gibberellins, growth inhibitors, morphactins, growth retardants, growth stimulators, and further unclassified plant growth regulators.

Suitable examples of antiauxins to be used in a composition according to the present invention are clofibric acid or 2,3,5-tri-iodobenzoic acid.

Suitable examples of auxins to be used in a composition according to the present invention are 4-CPA, 2,4-D, 2,4-DB, 2,4-DEP, dichlorprop, fenoprop, IAA (indole-3-acetic acid), IBA, naphthaleneacetamide, alpha-naphthaleneacetic acid, 1-naphthol, naphthoxyacetic acid, potassium naphthenate, sodium naphthenate or 2,4,5-T.

Suitable examples of cytokinins to be used in a composition according to the present invention are 2iP, 6-Benzylaminopurine (6-BA) (=N-6 Benzyladenine), 2,6-Dimethylpuridine (N-Oxide-2,6-Lultidine), 2,6-Dimethylpyridine, kinetin, or zeatin.

Suitable examples of defoliants to be used in a composition according to the present invention are calcium cyanamide, dimethipin, endothal, merphos, metoxuron, pentachlorophenol, thidiazuron, tribufos, or tributyl phosphorotrithioate.

Suitable examples of ethylene modulators to be used in a composition according to the present invention are aviglycine, 1-methylcyclopropene (1-MCP) Prohexadione (prohexadione calcium), or trinexapac (Trinexapac-ethyl).

Suitable examples of ethylene releasers to be used in a composition according to the present invention are ACC, etacelasil, ethephon, or glyoxime.

Suitable examples of gibberellins to be used in a composition according to the present invention are gibberelline or gibberellic acid.

Suitable examples of growth inhibitors to be used in a composition according to the present invention are abscisic acid, S-abscisic acid, ancymidol, butralin, carbaryl, chlorphonium, chlorpropham, dikegulac, flumetralin, fluoridamid, fosamine, glyphosine, isopyrimol, jasmonic acid, maleic hydrazide, mepiquat (mepiquat chloride, mepiquat pentaborate), piproctanyl, prohydrojasmon, propham, or 2,3,5-tri-iodobenzoic acid.

Suitable examples of morphactins to be used in a composition according to the present invention are chlorfluren, chlorflurenol, dichlorflurenol, or flurenol Suitable examples of growth retardants to be used in a composition according to the present invention are chlormequat (chlormequat chloride), daminozide, flurprimidol, mefluidide, paclobutrazol, tetcyclacis, uniconazole, metconazol.

Suitable examples of growth stimulators to be used in a composition according to the present invention are brassinolide, forchlorfenuron, or hymexazol.

Suitable examples of further unclassified plant growth regulators to be used in a composition according to the present invention are amidochlor, benzofluor, buminafos, carvone, choline chloride, ciobutide, clofencet, cloxyfonac, cyanamide, cyclanilide, cycloheximide, cyprosulfamide, epocholeone, ethychlozate, ethylene, fenridazon, fluprimidol, fluthiacet, heptopargil, holosulf, inabenfide, karetazan, lead arsenate, methasulfocarb, pydanon, sintofen, diflufenzopyr or triapenthenol

In a preferred embodiment, the composition according to the present invention may comprise a combination of the nitrification inhibitor of the compound of formula I and at least one compound selected from the group comprising: abscisic acid, amidochlor, ancymidol, 6-benzylaminopurine (=N-6 benzyladenine), brassinolide, butralin, chlormequat (chlormequat chloride), choline chloride, cyclanilide, daminozide, diflufenzopyr, dikegulac, dimethipin, 2,6-dimethylpyridine, ethephon, flumetralin, flurprimidol, fluthiacet, forchlorfenuron, gibberellic acid, inabenfide, indole-3-acetic acid, maleic hydrazide, mefluidide, mepiquat (mepiquat chloride), 1-methylcyclopropene (1-MCP), naphthaleneacetic acid, N-6 benzyladenine, paclobutrazol, prohexadione (prohexadione calcium), prohydrojasmon, thidiazuron, triapenthenol, tributyl phosphorotrithioate, 2,3,5-tri-iodobenzoic acid, trinexapac-ethyl, and uniconazole.

In a more preferred embodiment, the composition according to the present invention may comprise a combination of the nitrification inhibitor of the compound of formula I and at least one compound selected from the group comprising: 6-benzylaminopurine (=N-6 benzyladenine), chlormequat (chlormequat chloride), choline chloride, cyclanilide, diflufenzopyr, dikegulac, dimethipin, ethephon, flumetralin, fluthiacet, forchlorfenuron, gibberellic acid, inabenfide, maleic hydrazide, mepiquat (mepiquat chloride), 1-methylcyclopropene (1-MCP), paclobutrazol, prohexadione (prohexadione calcium), prohydrojasmon, thidiazuron, triapenthenol, tributyl phosphorotrithioate, trinexapac-ethyl, and uniconazole.

In the most preferred embodiment, the composition according to the present invention may comprise a combination of the nitrification inhibitor of the compound of formula I and at least one compound selected from the group comprising: chlormequat (chlormequat chloride) (P.1), choline chloride (P.2), cyclanilide (P.3), dimethipin (P.4), ethephon (P.5), forchlorfenuron (P.6), gibberellic acid (P.7), maleic hydrazide (P.8), mepiquat (mepiquat chloride) (P.9), 1-methylcyclopropene (1-MCP) (P.10), prohexadione (prohexadione calcium) (P.11), thidiazuron (P.12), and trinexapac-ethyl (P.13).

Accordingly, the present invention furthermore relates to compositions comprising as compound I (component A) a nitrification inhibitor as defined herein above and as compound II (component P) a plant growth regulator as defined herein, i.e. a combination of component A and P. In preferred embodiments, such compositions may be selected from the component A of Table 2 in column 2 (Com. A) and from component P of Table 2 in column 3 (Com. P). The number of component A of Table 2 corresponds to the number of component A as shown in Table 1, supra (“Entry”). Preferred embodiments thus include the specified combinations or compositions comprising component A and P as defined in Comb-1 to Comb-2795 of the following Table 2:

| ComP. | Com. A | Com. B |
|---|---|---|
| Comb-1 | 1 | P.1 |
| Comb-2 | 2 | P.1 |
| Comb-3 | 3 | P.1 |
| Comb-4 | 4 | P.1 |
| Comb-5 | 5 | P.1 |
| Comb-6 | 6 | P.1 |
| Comb-7 | 7 | P.1 |
| Comb-8 | 8 | P.1 |
| Comb-9 | 9 | P.1 |
| Comb-10 | 10 | P.1 |
| Comb-11 | 11 | P.1 |
| Comb-12 | 12 | P.1 |
| Comb-13 | 13 | P.1 |
| Comb-14 | 14 | P.1 |
| Comb-15 | 15 | P.1 |
| Comb-16 | 16 | P.1 |
| Comb-17 | 17 | P.1 |
| Comb-18 | 18 | P.1 |
| Comb-19 | 19 | P.1 |
| Comb-20 | 20 | P.1 |
| Comb-21 | 21 | P.1 |
| Comb-22 | 22 | P.1 |
| Comb-23 | 23 | P.1 |
| Comb-24 | 24 | P.1 |
| Comb-25 | 25 | P.1 |
| Comb-26 | 26 | P.1 |
| Comb-27 | 27 | P.1 |
| Comb-28 | 28 | P.1 |
| Comb-29 | 29 | P.1 |
| Comb-30 | 30 | P.1 |
| Comb-31 | 31 | P.1 |
| Comb-32 | 32 | P.1 |
| Comb-33 | 33 | P.1 |
| Comb-34 | 34 | P.1 |
| Comb-35 | 35 | P.1 |
| Comb-36 | 36 | P.1 |
| Comb-37 | 37 | P.1 |
| Comb-38 | 38 | P.1 |
| Comb-39 | 39 | P.1 |
| Comb-40 | 40 | P.1 |
| Comb-41 | 41 | P.1 |
| Comb-42 | 42 | P.1 |
| Comb-43 | 43 | P.1 |
| Comb-44 | 44 | P.1 |
| Comb-45 | 45 | P.1 |
| Comb-46 | 46 | P.1 |
| Comb-47 | 47 | P.1 |
| Comb-48 | 48 | P.1 |
| Comb-49 | 49 | P.1 |
| Comb-50 | 50 | P.1 |
| Comb-51 | 51 | P.1 |
| Comb-52 | 52 | P.1 |
| Comb-53 | 53 | P.1 |
| Comb-54 | 54 | P.1 |
| Comb-55 | 55 | P.1 |
| Comb-56 | 56 | P.1 |
| Comb-57 | 57 | P.1 |
| Comb-58 | 58 | P.1 |
| Comb-59 | 59 | P.1 |
| Comb-60 | 60 | P.1 |
| Comb-61 | 61 | P.1 |
| Comb-62 | 62 | P.1 |
| Comb-63 | 63 | P.1 |
| Comb-64 | 64 | P.1 |
| Comb-65 | 65 | P.1 |
| Comb-66 | 66 | P.1 |
| Comb-67 | 67 | P.1 |
| Comb-68 | 68 | P.1 |
| Comb-69 | 69 | P.1 |
| Comb-70 | 70 | P.1 |
| Comb-71 | 71 | P.1 |
| Comb-72 | 72 | P.1 |
| Comb-73 | 73 | P.1 |
| Comb-74 | 74 | P.1 |
| Comb-75 | 75 | P.1 |
| Comb-76 | 76 | P.1 |
| Comb-77 | 77 | P.1 |
| Comb-78 | 78 | P.1 |
| Comb-79 | 79 | P.1 |
| Comb-80 | 80 | P.1 |
| Comb-81 | 81 | P.1 |
| Comb-82 | 82 | P.1 |
| Comb-83 | 83 | P.1 |
| Comb-84 | 84 | P.1 |
| Comb-85 | 85 | P.1 |
| Comb-86 | 86 | P.1 |
| Comb-87 | 87 | P.1 |
| Comb-88 | 88 | P.1 |
| Comb-89 | 89 | P.1 |
| Comb-90 | 90 | P.1 |
| Comb-91 | 91 | P.1 |
| Comb-92 | 92 | P.1 |
| Comb-93 | 93 | P.1 |
| Comb-94 | 94 | P.1 |
| Comb-95 | 95 | P.1 |
| Comb-96 | 96 | P.1 |
| Comb-97 | 97 | P.1 |
| Comb-98 | 98 | P.1 |
| Comb-99 | 99 | P.1 |
| Comb-100 | 100 | P.1 |
| Comb-101 | 101 | P.1 |
| Comb-102 | 102 | P.1 |
| Comb-103 | 103 | P.1 |
| Comb-104 | 104 | P.1 |
| Comb-105 | 105 | P.1 |
| Comb-106 | 106 | P.1 |
| Comb-107 | 107 | P.1 |
| Comb-108 | 108 | P.1 |
| Comb-109 | 109 | P.1 |
| Comb-110 | 110 | P.1 |
| Comb-111 | 111 | P.1 |
| Comb-112 | 112 | P.1 |
| Comb-113 | 113 | P.1 |
| Comb-114 | 114 | P.1 |
| Comb-115 | 115 | P.1 |
| Comb-116 | 116 | P.1 |
| Comb-117 | 117 | P.1 |
| Comb-118 | 118 | P.1 |
| Comb-119 | 119 | P.1 |
| Comb-120 | 120 | P.1 |
| Comb-121 | 121 | P.1 |
| Comb-122 | 122 | P.1 |
| Comb-123 | 123 | P.1 |
| Comb-124 | 124 | P.1 |
| Comb-125 | 125 | P.1 |
| Comb-126 | 126 | P.1 |
| Comb-127 | 127 | P.1 |
| Comb-128 | 128 | P.1 |
| Comb-129 | 129 | P.1 |
| Comb-130 | 130 | P.1 |
| Comb-131 | 131 | P.1 |
| Comb-132 | 132 | P.1 |
| Comb-133 | 133 | P.1 |
| Comb-134 | 134 | P.1 |
| Comb-135 | 135 | P.1 |
| Comb-136 | 136 | P.1 |
| Comb-137 | 137 | P.1 |
| Comb-138 | 138 | P.1 |
| Comb-139 | 139 | P.1 |
| Comb-140 | 140 | P.1 |
| Comb-141 | 141 | P.1 |
| Comb-142 | 142 | P.1 |
| Comb-143 | 143 | P.1 |
| Comb-144 | 144 | P.1 |
| Comb-145 | 145 | P.1 |
| Comb-146 | 146 | P.1 |
| Comb-147 | 147 | P.1 |
| Comb-148 | 148 | P.1 |
| Comb-149 | 149 | P.1 |
| Comb-150 | 150 | P.1 |
| Comb-151 | 151 | P.1 |
| Comb-152 | 152 | P.1 |
| Comb-153 | 153 | P.1 |
| Comb-154 | 154 | P.1 |
| Comb-155 | 155 | P.1 |
| Comb-156 | 156 | P.1 |
| Comb-157 | 157 | P.1 |
| Comb-158 | 158 | P.1 |
| Comb-159 | 159 | P.1 |
| Comb-160 | 160 | P.1 |
| Comb-161 | 161 | P.1 |
| Comb-162 | 162 | P.1 |
| Comb-163 | 163 | P.1 |
| Comb-164 | 164 | P.1 |
| Comb-165 | 165 | P.1 |
| Comb-166 | 166 | P.1 |
| Comb-167 | 167 | P.1 |
| Comb-168 | 168 | P.1 |
| Comb-169 | 169 | P.1 |
| Comb-170 | 170 | P.1 |
| Comb-171 | 171 | P.1 |
| Comb-172 | 172 | P.1 |
| Comb-173 | 173 | P.1 |
| Comb-174 | 174 | P.1 |
| Comb-175 | 175 | P.1 |
| Comb-176 | 176 | P.1 |
| Comb-177 | 177 | P.1 |
| Comb-178 | 178 | P.1 |
| Comb-179 | 179 | P.1 |
| Comb-180 | 180 | P.1 |
| Comb-181 | 181 | P.1 |
| Comb-182 | 182 | P.1 |
| Comb-183 | 183 | P.1 |
| Comb-184 | 184 | P.1 |
| Comb-185 | 185 | P.1 |
| Comb-186 | 186 | P.1 |
| Comb-187 | 187 | P.1 |
| Comb-188 | 188 | P.1 |
| Comb-189 | 189 | P.1 |
| Comb-190 | 190 | P.1 |
| Comb-191 | 191 | P.1 |
| Comb-192 | 192 | P.1 |
| Comb-193 | 193 | P.1 |
| Comb-194 | 194 | P.1 |
| Comb-195 | 195 | P.1 |
| Comb-196 | 196 | P.1 |
| Comb-197 | 197 | P.1 |
| Comb-198 | 198 | P.1 |
| Comb-199 | 199 | P.1 |
| Comb-200 | 200 | P.1 |
| Comb-201 | 201 | P.1 |
| Comb-202 | 202 | P.1 |
| Comb-203 | 203 | P.1 |
| Comb-204 | 204 | P.1 |
| Comb-205 | 205 | P.1 |
| Comb-206 | 206 | P.1 |
| Comb-207 | 207 | P.1 |
| Comb-208 | 208 | P.1 |
| Comb-209 | 209 | P.1 |
| Comb-210 | 210 | P.1 |
| Comb-211 | 211 | P.1 |
| Comb-212 | 212 | P.1 |
| Comb-213 | 213 | P.1 |
| Comb-214 | 214 | P.1 |
| Comb-215 | 215 | P.1 |
| Comb-216 | 1 | P.2 |
| Comb-217 | 2 | P.2 |
| Comb-218 | 3 | P.2 |
| Comb-219 | 4 | P.2 |
| Comb-220 | 5 | P.2 |
| Comb-221 | 6 | P.2 |
| Comb-222 | 7 | P.2 |
| Comb-223 | 8 | P.2 |
| Comb-224 | 9 | P.2 |
| Comb-225 | 10 | P.2 |
| Comb-226 | 11 | P.2 |
| Comb-227 | 12 | P.2 |
| Comb-228 | 13 | P.2 |
| Comb-229 | 14 | P.2 |
| Comb-230 | 15 | P.2 |
| Comb-231 | 16 | P.2 |
| Comb-232 | 17 | P.2 |
| Comb-233 | 18 | P.2 |
| Comb-234 | 19 | P.2 |
| Comb-235 | 20 | P.2 |
| Comb-236 | 21 | P.2 |
| Comb-237 | 22 | P.2 |
| Comb-238 | 23 | P.2 |
| Comb-239 | 24 | P.2 |
| Comb-240 | 25 | P.2 |
| Comb-241 | 26 | P.2 |
| Comb-242 | 27 | P.2 |
| Comb-243 | 28 | P.2 |
| Comb-244 | 29 | P.2 |
| Comb-245 | 30 | P.2 |
| Comb-246 | 31 | P.2 |
| Comb-247 | 32 | P.2 |
| Comb-248 | 33 | P.2 |
| Comb-249 | 34 | P.2 |
| Comb-250 | 35 | P.2 |
| Comb-251 | 36 | P.2 |
| Comb-252 | 37 | P.2 |
| Comb-253 | 38 | P.2 |
| Comb-254 | 39 | P.2 |
| Comb-255 | 40 | P.2 |
| Comb-256 | 41 | P.2 |
| Comb-257 | 42 | P.2 |
| Comb-258 | 43 | P.2 |
| Comb-259 | 44 | P.2 |
| Comb-260 | 45 | P.2 |
| Comb-261 | 46 | P.2 |
| Comb-262 | 47 | P.2 |
| Comb-263 | 48 | P.2 |
| Comb-264 | 49 | P.2 |
| Comb-265 | 50 | P.2 |
| Comb-266 | 51 | P.2 |
| Comb-267 | 52 | P.2 |
| Comb-268 | 53 | P.2 |
| Comb-269 | 54 | P.2 |
| Comb-270 | 55 | P.2 |
| Comb-271 | 56 | P.2 |
| Comb-272 | 57 | P.2 |
| Comb-273 | 58 | P.2 |
| Comb-274 | 59 | P.2 |
| Comb-275 | 60 | P.2 |
| Comb-276 | 61 | P.2 |
| Comb-277 | 62 | P.2 |
| Comb-278 | 63 | P.2 |
| Comb-279 | 64 | P.2 |
| Comb-280 | 65 | P.2 |
| Comb-281 | 66 | P.2 |
| Comb-282 | 67 | P.2 |
| Comb-283 | 68 | P.2 |
| Comb-284 | 69 | P.2 |
| Comb-285 | 70 | P.2 |
| Comb-286 | 71 | P.2 |
| Comb-287 | 72 | P.2 |
| Comb-288 | 73 | P.2 |
| Comb-289 | 74 | P.2 |
| Comb-290 | 75 | P.2 |
| Comb-291 | 76 | P.2 |
| Comb-292 | 77 | P.2 |
| Comb-293 | 78 | P.2 |
| Comb-294 | 79 | P.2 |
| Comb-295 | 80 | P.2 |
| Comb-296 | 81 | P.2 |
| Comb-297 | 82 | P.2 |
| Comb-298 | 83 | P.2 |
| Comb-299 | 84 | P.2 |
| Comb-300 | 85 | P.2 |
| Comb-301 | 86 | P.2 |
| Comb-302 | 87 | P.2 |
| Comb-303 | 88 | P.2 |
| Comb-304 | 89 | P.2 |
| Comb-305 | 90 | P.2 |
| Comb-306 | 91 | P.2 |
| Comb-307 | 92 | P.2 |
| Comb-308 | 93 | P.2 |
| Comb-309 | 94 | P.2 |
| Comb-310 | 95 | P.2 |
| Comb-311 | 96 | P.2 |
| Comb-312 | 97 | P.2 |
| Comb-313 | 98 | P.2 |
| Comb-314 | 99 | P.2 |
| Comb-315 | 100 | P.2 |
| Comb-316 | 101 | P.2 |
| Comb-317 | 102 | P.2 |
| Comb-318 | 103 | P.2 |
| Comb-319 | 104 | P.2 |
| Comb-320 | 105 | P.2 |
| Comb-321 | 106 | P.2 |
| Comb-322 | 107 | P.2 |
| Comb-323 | 108 | P.2 |
| Comb-324 | 109 | P.2 |
| Comb-325 | 110 | P.2 |
| Comb-326 | 111 | P.2 |
| Comb-327 | 112 | P.2 |
| Comb-328 | 113 | P.2 |
| Comb-329 | 114 | P.2 |
| Comb-330 | 115 | P.2 |
| Comb-331 | 116 | P.2 |
| Comb-332 | 117 | P.2 |
| Comb-333 | 118 | P.2 |
| Comb-334 | 119 | P.2 |
| Comb-335 | 120 | P.2 |
| Comb-336 | 121 | P.2 |
| Comb-337 | 122 | P.2 |
| Comb-338 | 123 | P.2 |
| Comb-339 | 124 | P.2 |
| Comb-340 | 125 | P.2 |
| Comb-341 | 126 | P.2 |
| Comb-342 | 127 | P.2 |
| Comb-343 | 128 | P.2 |
| Comb-344 | 129 | P.2 |
| Comb-345 | 130 | P.2 |
| Comb-346 | 131 | P.2 |
| Comb-347 | 132 | P.2 |
| Comb-348 | 133 | P.2 |
| Comb-349 | 134 | P.2 |
| Comb-350 | 135 | P.2 |
| Comb-351 | 136 | P.2 |
| Comb-352 | 137 | P.2 |
| Comb-353 | 138 | P.2 |
| Comb-354 | 139 | P.2 |
| Comb-355 | 140 | P.2 |
| Comb-356 | 141 | P.2 |
| Comb-357 | 142 | P.2 |
| Comb-358 | 143 | P.2 |
| Comb-359 | 144 | P.2 |
| Comb-360 | 145 | P.2 |
| Comb-361 | 146 | P.2 |
| Comb-362 | 147 | P.2 |
| Comb-363 | 148 | P.2 |
| Comb-364 | 149 | P.2 |
| Comb-365 | 150 | P.2 |
| Comb-366 | 151 | P.2 |
| Comb-367 | 152 | P.2 |
| Comb-368 | 153 | P.2 |
| Comb-369 | 154 | P.2 |
| Comb-370 | 155 | P.2 |
| Comb-371 | 156 | P.2 |
| Comb-372 | 157 | P.2 |
| Comb-373 | 158 | P.2 |
| Comb-374 | 159 | P.2 |
| Comb-375 | 160 | P.2 |
| Comb-376 | 161 | P.2 |
| Comb-377 | 162 | P.2 |
| Comb-378 | 163 | P.2 |
| Comb-379 | 164 | P.2 |
| Comb-380 | 165 | P.2 |
| Comb-381 | 166 | P.2 |
| Comb-382 | 167 | P.2 |
| Comb-383 | 168 | P.2 |
| Comb-384 | 169 | P.2 |
| Comb-385 | 170 | P.2 |
| Comb-386 | 171 | P.2 |
| Comb-387 | 172 | P.2 |
| Comb-388 | 173 | P.2 |
| Comb-389 | 174 | P.2 |
| Comb-390 | 175 | P.2 |
| Comb-391 | 176 | P.2 |
| Comb-392 | 177 | P.2 |
| Comb-393 | 178 | P.2 |
| Comb-394 | 179 | P.2 |
| Comb-395 | 180 | P.2 |
| Comb-396 | 181 | P.2 |
| Comb-397 | 182 | P.2 |
| Comb-398 | 183 | P.2 |
| Comb-399 | 184 | P.2 |
| Comb-400 | 185 | P.2 |
| Comb-401 | 186 | P.2 |
| Comb-402 | 187 | P.2 |
| Comb-403 | 188 | P.2 |
| Comb-404 | 189 | P.2 |
| Comb-405 | 190 | P.2 |
| Comb-406 | 191 | P.2 |
| Comb-407 | 192 | P.2 |
| Comb-408 | 193 | P.2 |
| Comb-409 | 194 | P.2 |
| Comb-410 | 195 | P.2 |
| Comb-411 | 196 | P.2 |
| Comb-412 | 197 | P.2 |
| Comb-413 | 198 | P.2 |
| Comb-414 | 199 | P.2 |
| Comb-415 | 200 | P.2 |
| Comb-416 | 201 | P.2 |
| Comb-417 | 202 | P.2 |
| Comb-418 | 203 | P.2 |
| Comb-419 | 204 | P.2 |
| Comb-420 | 205 | P.2 |
| Comb-421 | 206 | P.2 |
| Comb-422 | 207 | P.2 |
| Comb-423 | 208 | P.2 |
| Comb-424 | 209 | P.2 |
| Comb-425 | 210 | P.2 |
| Comb-426 | 211 | P.2 |
| Comb-427 | 212 | P.2 |
| Comb-428 | 213 | P.2 |
| Comb-429 | 214 | P.2 |
| Comb-430 | 215 | P.2 |
| Comb-431 | 1 | P.3 |
| Comb-432 | 2 | P.3 |
| Comb-433 | 3 | P.3 |
| Comb-434 | 4 | P.3 |
| Comb-435 | 5 | P.3 |
| Comb-436 | 6 | P.3 |
| Comb-437 | 7 | P.3 |
| Comb-438 | 8 | P.3 |
| Comb-439 | 9 | P.3 |
| Comb-440 | 10 | P.3 |
| Comb-441 | 11 | P.3 |
| Comb-442 | 12 | P.3 |
| Comb-443 | 13 | P.3 |
| Comb-444 | 14 | P.3 |
| Comb-445 | 15 | P.3 |
| Comb-446 | 16 | P.3 |
| Comb-447 | 17 | P.3 |
| Comb-448 | 18 | P.3 |
| Comb-449 | 19 | P.3 |
| Comb-450 | 20 | P.3 |
| Comb-451 | 21 | P.3 |
| Comb-452 | 22 | P.3 |
| Comb-453 | 23 | P.3 |
| Comb-454 | 24 | P.3 |
| Comb-455 | 25 | P.3 |
| Comb-456 | 26 | P.3 |
| Comb-457 | 27 | P.3 |
| Comb-458 | 28 | P.3 |
| Comb-459 | 29 | P.3 |
| Comb-460 | 30 | P.3 |
| Comb-461 | 31 | P.3 |
| Comb-462 | 32 | P.3 |
| Comb-463 | 33 | P.3 |
| Comb-464 | 34 | P.3 |
| Comb-465 | 35 | P.3 |
| Comb-466 | 36 | P.3 |
| Comb-467 | 37 | P.3 |
| Comb-468 | 38 | P.3 |
| Comb-469 | 39 | P.3 |
| Comb-470 | 40 | P.3 |
| Comb-471 | 41 | P.3 |
| Comb-472 | 42 | P.3 |
| Comb-473 | 43 | P.3 |
| Comb-474 | 44 | P.3 |
| Comb-475 | 45 | P.3 |
| Comb-476 | 46 | P.3 |
| Comb-477 | 47 | P.3 |
| Comb-478 | 48 | P.3 |
| Comb-479 | 49 | P.3 |
| Comb-480 | 50 | P.3 |
| Comb-481 | 51 | P.3 |
| Comb-482 | 52 | P.3 |
| Comb-483 | 53 | P.3 |
| Comb-484 | 54 | P.3 |
| Comb-485 | 55 | P.3 |
| Comb-486 | 56 | P.3 |
| Comb-487 | 57 | P.3 |
| Comb-488 | 58 | P.3 |
| Comb-489 | 59 | P.3 |
| Comb-490 | 60 | P.3 |
| Comb-491 | 61 | P.3 |
| Comb-492 | 62 | P.3 |
| Comb-493 | 63 | P.3 |
| Comb-494 | 64 | P.3 |
| Comb-495 | 65 | P.3 |
| Comb-496 | 66 | P.3 |
| Comb-497 | 67 | P.3 |
| Comb-498 | 68 | P.3 |
| Comb-499 | 69 | P.3 |
| Comb-500 | 70 | P.3 |
| Comb-501 | 71 | P.3 |
| Comb-502 | 72 | P.3 |
| Comb-503 | 73 | P.3 |
| Comb-504 | 74 | P.3 |
| Comb-505 | 75 | P.3 |
| Comb-506 | 76 | P.3 |
| Comb-507 | 77 | P.3 |
| Comb-508 | 78 | P.3 |
| Comb-509 | 79 | P.3 |
| Comb-510 | 80 | P.3 |
| Comb-511 | 81 | P.3 |
| Comb-512 | 82 | P.3 |
| Comb-513 | 83 | P.3 |
| Comb-514 | 84 | P.3 |
| Comb-515 | 85 | P.3 |
| Comb-516 | 86 | P.3 |
| Comb-517 | 87 | P.3 |
| Comb-518 | 88 | P.3 |
| Comb-519 | 89 | P.3 |
| Comb-520 | 90 | P.3 |
| Comb-521 | 91 | P.3 |
| Comb-522 | 92 | P.3 |
| Comb-523 | 93 | P.3 |
| Comb-524 | 94 | P.3 |
| Comb-525 | 95 | P.3 |
| Comb-526 | 96 | P.3 |
| Comb-527 | 97 | P.3 |
| Comb-528 | 98 | P.3 |
| Comb-529 | 99 | P.3 |
| Comb-530 | 100 | P.3 |
| Comb-531 | 101 | P.3 |
| Comb-532 | 102 | P.3 |
| Comb-533 | 103 | P.3 |
| Comb-534 | 104 | P.3 |
| Comb-535 | 105 | P.3 |
| Comb-536 | 106 | P.3 |
| Comb-537 | 107 | P.3 |
| Comb-538 | 108 | P.3 |
| Comb-539 | 109 | P.3 |
| Comb-540 | 110 | P.3 |
| Comb-541 | 111 | P.3 |
| Comb-542 | 112 | P.3 |
| Comb-543 | 113 | P.3 |
| Comb-544 | 114 | P.3 |
| Comb-545 | 115 | P.3 |
| Comb-546 | 116 | P.3 |
| Comb-547 | 117 | P.3 |
| Comb-548 | 118 | P.3 |
| Comb-549 | 119 | P.3 |
| Comb-550 | 120 | P.3 |
| Comb-551 | 121 | P.3 |
| Comb-552 | 122 | P.3 |
| Comb-553 | 123 | P.3 |
| Comb-554 | 124 | P.3 |
| Comb-555 | 125 | P.3 |
| Comb-556 | 126 | P.3 |
| Comb-557 | 127 | P.3 |
| Comb-558 | 128 | P.3 |
| Comb-559 | 129 | P.3 |
| Comb-560 | 130 | P.3 |
| Comb-561 | 131 | P.3 |
| Comb-562 | 132 | P.3 |
| Comb-563 | 133 | P.3 |
| Comb-564 | 134 | P.3 |
| Comb-565 | 135 | P.3 |
| Comb-566 | 136 | P.3 |
| Comb-567 | 137 | P.3 |
| Comb-568 | 138 | P.3 |
| Comb-569 | 139 | P.3 |
| Comb-570 | 140 | P.3 |
| Comb-571 | 141 | P.3 |
| Comb-572 | 142 | P.3 |
| Comb-573 | 143 | P.3 |
| Comb-574 | 144 | P.3 |
| Comb-575 | 145 | P.3 |
| Comb-576 | 146 | P.3 |
| Comb-577 | 147 | P.3 |
| Comb-578 | 148 | P.3 |
| Comb-579 | 149 | P.3 |
| Comb-580 | 150 | P.3 |
| Comb-581 | 151 | P.3 |
| Comb-582 | 152 | P.3 |
| Comb-583 | 153 | P.3 |
| Comb-584 | 154 | P.3 |
| Comb-585 | 155 | P.3 |
| Comb-586 | 156 | P.3 |
| Comb-587 | 157 | P.3 |
| Comb-588 | 158 | P.3 |
| Comb-589 | 159 | P.3 |
| Comb-590 | 160 | P.3 |
| Comb-591 | 161 | P.3 |
| Comb-592 | 162 | P.3 |
| Comb-593 | 163 | P.3 |
| Comb-594 | 164 | P.3 |
| Comb-595 | 165 | P.3 |
| Comb-596 | 166 | P.3 |
| Comb-597 | 167 | P.3 |
| Comb-598 | 168 | P.3 |
| Comb-599 | 169 | P.3 |
| Comb-600 | 170 | P.3 |
| Comb-601 | 171 | P.3 |
| Comb-602 | 172 | P.3 |
| Comb-603 | 173 | P.3 |
| Comb-604 | 174 | P.3 |
| Comb-605 | 175 | P.3 |
| Comb-606 | 176 | P.3 |
| Comb-607 | 177 | P.3 |
| Comb-608 | 178 | P.3 |
| Comb-609 | 179 | P.3 |
| Comb-610 | 180 | P.3 |
| Comb-611 | 181 | P.3 |
| Comb-612 | 182 | P.3 |
| Comb-613 | 183 | P.3 |
| Comb-614 | 184 | P.3 |
| Comb-615 | 185 | P.3 |
| Comb-616 | 186 | P.3 |
| Comb-617 | 187 | P.3 |
| Comb-618 | 188 | P.3 |
| Comb-619 | 189 | P.3 |
| Comb-620 | 190 | P.3 |
| Comb-621 | 191 | P.3 |
| Comb-622 | 192 | P.3 |
| Comb-623 | 193 | P.3 |
| Comb-624 | 194 | P.3 |
| Comb-625 | 195 | P.3 |
| Comb-626 | 196 | P.3 |
| Comb-627 | 197 | P.3 |
| Comb-628 | 198 | P.3 |
| Comb-629 | 199 | P.3 |
| Comb-630 | 200 | P.3 |
| Comb-631 | 201 | P.3 |
| Comb-632 | 202 | P.3 |
| Comb-633 | 203 | P.3 |
| Comb-634 | 204 | P.3 |
| Comb-635 | 205 | P.3 |
| Comb-636 | 206 | P.3 |
| Comb-637 | 207 | P.3 |
| Comb-638 | 208 | P.3 |
| Comb-639 | 209 | P.3 |
| Comb-640 | 210 | P.3 |
| Comb-641 | 211 | P.3 |
| Comb-642 | 212 | P.3 |
| Comb-643 | 213 | P.3 |
| Comb-644 | 214 | P.3 |
| Comb-645 | 215 | P.3 |
| Comb-646 | 1 | P.4 |
| Comb-647 | 2 | P.4 |
| Comb-648 | 3 | P.4 |
| Comb-649 | 4 | P.4 |
| Comb-650 | 5 | P.4 |
| Comb-651 | 6 | P.4 |
| Comb-652 | 7 | P.4 |
| Comb-653 | 8 | P.4 |
| Comb-654 | 9 | P.4 |
| Comb-655 | 10 | P.4 |
| Comb-656 | 11 | P.4 |
| Comb-657 | 12 | P.4 |
| Comb-658 | 13 | P.4 |
| Comb-659 | 14 | P.4 |
| Comb-660 | 15 | P.4 |
| Comb-661 | 16 | P.4 |
| Comb-662 | 17 | P.4 |
| Comb-663 | 18 | P.4 |
| Comb-664 | 19 | P.4 |
| Comb-665 | 20 | P.4 |
| Comb-666 | 21 | P.4 |
| Comb-667 | 22 | P.4 |
| Comb-668 | 23 | P.4 |
| Comb-669 | 24 | P.4 |
| Comb-670 | 25 | P.4 |
| Comb-671 | 26 | P.4 |
| Comb-672 | 27 | P.4 |
| Comb-673 | 28 | P.4 |
| Comb-674 | 29 | P.4 |
| Comb-675 | 30 | P.4 |
| Comb-676 | 31 | P.4 |
| Comb-677 | 32 | P.4 |
| Comb-678 | 33 | P.4 |
| Comb-679 | 34 | P.4 |
| Comb-680 | 35 | P.4 |
| Comb-681 | 36 | P.4 |
| Comb-682 | 37 | P.4 |
| Comb-683 | 38 | P.4 |
| Comb-684 | 39 | P.4 |
| Comb-685 | 40 | P.4 |
| Comb-686 | 41 | P.4 |
| Comb-687 | 42 | P.4 |
| Comb-688 | 43 | P.4 |
| Comb-689 | 44 | P.4 |
| Comb-690 | 45 | P.4 |
| Comb-691 | 46 | P.4 |
| Comb-692 | 47 | P.4 |
| Comb-693 | 48 | P.4 |
| Comb-694 | 49 | P.4 |
| Comb-695 | 50 | P.4 |
| Comb-696 | 51 | P.4 |
| Comb-697 | 52 | P.4 |
| Comb-698 | 53 | P.4 |
| Comb-699 | 54 | P.4 |
| Comb-700 | 55 | P.4 |
| Comb-701 | 56 | P.4 |
| Comb-702 | 57 | P.4 |
| Comb-703 | 58 | P.4 |
| Comb-704 | 59 | P.4 |
| Comb-705 | 60 | P.4 |
| Comb-706 | 61 | P.4 |
| Comb-707 | 62 | P.4 |
| Comb-708 | 63 | P.4 |
| Comb-709 | 64 | P.4 |
| Comb-710 | 65 | P.4 |
| Comb-711 | 66 | P.4 |
| Comb-712 | 67 | P.4 |
| Comb-713 | 68 | P.4 |
| Comb-714 | 69 | P.4 |
| Comb-715 | 70 | P.4 |
| Comb-716 | 71 | P.4 |
| Comb-717 | 72 | P.4 |
| Comb-718 | 73 | P.4 |
| Comb-719 | 74 | P.4 |
| Comb-720 | 75 | P.4 |
| Comb-721 | 76 | P.4 |
| Comb-722 | 77 | P.4 |
| Comb-723 | 78 | P.4 |
| Comb-724 | 79 | P.4 |
| Comb-725 | 80 | P.4 |
| Comb-726 | 81 | P.4 |
| Comb-727 | 82 | P.4 |
| Comb-728 | 83 | P.4 |
| Comb-729 | 84 | P.4 |
| Comb-730 | 85 | P.4 |
| Comb-731 | 86 | P.4 |
| Comb-732 | 87 | P.4 |
| Comb-733 | 88 | P.4 |
| Comb-734 | 89 | P.4 |
| Comb-735 | 90 | P.4 |
| Comb-736 | 91 | P.4 |
| Comb-737 | 92 | P.4 |
| Comb-738 | 93 | P.4 |
| Comb-739 | 94 | P.4 |
| Comb-740 | 95 | P.4 |
| Comb-741 | 96 | P.4 |
| Comb-742 | 97 | P.4 |
| Comb-743 | 98 | P.4 |
| Comb-744 | 99 | P.4 |
| Comb-745 | 100 | P.4 |
| Comb-746 | 101 | P.4 |
| Comb-747 | 102 | P.4 |
| Comb-748 | 103 | P.4 |
| Comb-749 | 104 | P.4 |
| Comb-750 | 105 | P.4 |
| Comb-751 | 106 | P.4 |
| Comb-752 | 107 | P.4 |
| Comb-753 | 108 | P.4 |
| Comb-754 | 109 | P.4 |
| Comb-755 | 110 | P.4 |
| Comb-756 | 111 | P.4 |
| Comb-757 | 112 | P.4 |
| Comb-758 | 113 | P.4 |
| Comb-759 | 114 | P.4 |
| Comb-760 | 115 | P.4 |
| Comb-761 | 116 | P.4 |
| Comb-762 | 117 | P.4 |
| Comb-763 | 118 | P.4 |
| Comb-764 | 119 | P.4 |
| Comb-765 | 120 | P.4 |
| Comb-766 | 121 | P.4 |
| Comb-767 | 122 | P.4 |
| Comb-768 | 123 | P.4 |
| Comb-769 | 124 | P.4 |
| Comb-770 | 125 | P.4 |
| Comb-771 | 126 | P.4 |
| Comb-772 | 127 | P.4 |
| Comb-773 | 128 | P.4 |
| Comb-774 | 129 | P.4 |
| Comb-775 | 130 | P.4 |
| Comb-776 | 131 | P.4 |
| Comb-777 | 132 | P.4 |
| Comb-778 | 133 | P.4 |
| Comb-779 | 134 | P.4 |
| Comb-780 | 135 | P.4 |
| Comb-781 | 136 | P.4 |
| Comb-782 | 137 | P.4 |
| Comb-783 | 138 | P.4 |
| Comb-784 | 139 | P.4 |
| Comb-785 | 140 | P.4 |
| Comb-786 | 141 | P.4 |
| Comb-787 | 142 | P.4 |
| Comb-788 | 143 | P.4 |
| Comb-789 | 144 | P.4 |
| Comb-790 | 145 | P.4 |
| Comb-791 | 146 | P.4 |
| Comb-792 | 147 | P.4 |
| Comb-793 | 148 | P.4 |
| Comb-794 | 149 | P.4 |
| Comb-795 | 150 | P.4 |
| Comb-796 | 151 | P.4 |
| Comb-797 | 152 | P.4 |
| Comb-798 | 153 | P.4 |
| Comb-799 | 154 | P.4 |
| Comb-800 | 155 | P.4 |
| Comb-801 | 156 | P.4 |
| Comb-802 | 157 | P.4 |
| Comb-803 | 158 | P.4 |
| Comb-804 | 159 | P.4 |
| Comb-805 | 160 | P.4 |
| Comb-806 | 161 | P.4 |
| Comb-807 | 162 | P.4 |
| Comb-808 | 163 | P.4 |
| Comb-809 | 164 | P.4 |
| Comb-810 | 165 | P.4 |
| Comb-811 | 166 | P.4 |
| Comb-812 | 167 | P.4 |
| Comb-813 | 168 | P.4 |
| Comb-814 | 169 | P.4 |
| Comb-815 | 170 | P.4 |
| Comb-816 | 171 | P.4 |
| Comb-817 | 172 | P.4 |
| Comb-818 | 173 | P.4 |
| Comb-819 | 174 | P.4 |
| Comb-820 | 175 | P.4 |
| Comb-821 | 176 | P.4 |
| Comb-822 | 177 | P.4 |
| Comb-823 | 178 | P.4 |
| Comb-824 | 179 | P.4 |
| Comb-825 | 180 | P.4 |
| Comb-826 | 181 | P.4 |
| Comb-827 | 182 | P.4 |
| Comb-828 | 183 | P.4 |
| Comb-829 | 184 | P.4 |
| Comb-830 | 185 | P.4 |
| Comb-831 | 186 | P.4 |
| Comb-832 | 187 | P.4 |
| Comb-833 | 188 | P.4 |
| Comb-834 | 189 | P.4 |
| Comb-835 | 190 | P.4 |
| Comb-836 | 191 | P.4 |
| Comb-837 | 192 | P.4 |
| Comb-838 | 193 | P.4 |
| Comb-839 | 194 | P.4 |
| Comb-840 | 195 | P.4 |
| Comb-841 | 196 | P.4 |
| Comb-842 | 197 | P.4 |
| Comb-843 | 198 | P.4 |
| Comb-844 | 199 | P.4 |
| Comb-845 | 200 | P.4 |
| Comb-846 | 201 | P.4 |
| Comb-847 | 202 | P.4 |
| Comb-848 | 203 | P.4 |
| Comb-849 | 204 | P.4 |
| Comb-850 | 205 | P.4 |
| Comb-851 | 206 | P.4 |
| Comb-852 | 207 | P.4 |
| Comb-853 | 208 | P.4 |
| Comb-854 | 209 | P.4 |
| Comb-855 | 210 | P.4 |
| Comb-856 | 211 | P.4 |
| Comb-857 | 212 | P.4 |
| Comb-858 | 213 | P.4 |
| Comb-859 | 214 | P.4 |
| Comb-860 | 215 | P.4 |
| Comb-861 | 1 | P.5 |
| Comb-862 | 2 | P.5 |
| Comb-863 | 3 | P.5 |
| Comb-864 | 4 | P.5 |
| Comb-865 | 5 | P.5 |
| Comb-866 | 6 | P.5 |
| Comb-867 | 7 | P.5 |
| Comb-868 | 8 | P.5 |
| Comb-869 | 9 | P.5 |
| Comb-870 | 10 | P.5 |
| Comb-871 | 11 | P.5 |
| Comb-872 | 12 | P.5 |
| Comb-873 | 13 | P.5 |
| Comb-874 | 14 | P.5 |
| Comb-875 | 15 | P.5 |
| Comb-876 | 16 | P.5 |
| Comb-877 | 17 | P.5 |
| Comb-878 | 18 | P.5 |
| Comb-879 | 19 | P.5 |
| Comb-880 | 20 | P.5 |
| Comb-881 | 21 | P.5 |
| Comb-882 | 22 | P.5 |
| Comb-883 | 23 | P.5 |
| Comb-884 | 24 | P.5 |
| Comb-885 | 25 | P.5 |
| Comb-886 | 26 | P.5 |
| Comb-887 | 27 | P.5 |
| Comb-888 | 28 | P.5 |
| Comb-889 | 29 | P.5 |
| Comb-890 | 30 | P.5 |
| Comb-891 | 31 | P.5 |
| Comb-892 | 32 | P.5 |
| Comb-893 | 33 | P.5 |
| Comb-894 | 34 | P.5 |
| Comb-895 | 35 | P.5 |
| Comb-896 | 36 | P.5 |
| Comb-897 | 37 | P.5 |
| Comb-898 | 38 | P.5 |
| Comb-899 | 39 | P.5 |
| Comb-900 | 40 | P.5 |
| Comb-901 | 41 | P.5 |
| Comb-902 | 42 | P.5 |
| Comb-903 | 43 | P.5 |
| Comb-904 | 44 | P.5 |
| Comb-905 | 45 | P.5 |
| Comb-906 | 46 | P.5 |
| Comb-907 | 47 | P.5 |
| Comb-908 | 48 | P.5 |
| Comb-909 | 49 | P.5 |
| Comb-910 | 50 | P.5 |
| Comb-911 | 51 | P.5 |
| Comb-912 | 52 | P.5 |
| Comb-913 | 53 | P.5 |
| Comb-914 | 54 | P.5 |
| Comb-915 | 55 | P.5 |
| Comb-916 | 56 | P.5 |
| Comb-917 | 57 | P.5 |
| Comb-918 | 58 | P.5 |
| Comb-919 | 59 | P.5 |
| Comb-920 | 60 | P.5 |
| Comb-921 | 61 | P.5 |
| Comb-922 | 62 | P.5 |
| Comb-923 | 63 | P.5 |
| Comb-924 | 64 | P.5 |
| Comb-925 | 65 | P.5 |
| Comb-926 | 66 | P.5 |
| Comb-927 | 67 | P.5 |
| Comb-928 | 68 | P.5 |
| Comb-929 | 69 | P.5 |
| Comb-930 | 70 | P.5 |
| Comb-931 | 71 | P.5 |
| Comb-932 | 72 | P.5 |
| Comb-933 | 73 | P.5 |
| Comb-934 | 74 | P.5 |
| Comb-935 | 75 | P.5 |
| Comb-936 | 76 | P.5 |
| Comb-937 | 77 | P.5 |
| Comb-938 | 78 | P.5 |
| Comb-939 | 79 | P.5 |
| Comb-940 | 80 | P.5 |
| Comb-941 | 81 | P.5 |
| Comb-942 | 82 | P.5 |
| Comb-943 | 83 | P.5 |
| Comb-944 | 84 | P.5 |
| Comb-945 | 85 | P.5 |
| Comb-946 | 86 | P.5 |
| Comb-947 | 87 | P.5 |
| Comb-948 | 88 | P.5 |
| Comb-949 | 89 | P.5 |
| Comb-950 | 90 | P.5 |
| Comb-951 | 91 | P.5 |
| Comb-952 | 92 | P.5 |
| Comb-953 | 93 | P.5 |
| Comb-954 | 94 | P.5 |
| Comb-955 | 95 | P.5 |
| Comb-956 | 96 | P.5 |
| Comb-957 | 97 | P.5 |
| Comb-958 | 98 | P.5 |
| Comb-959 | 99 | P.5 |
| Comb-960 | 100 | P.5 |
| Comb-961 | 101 | P.5 |
| Comb-962 | 102 | P.5 |
| Comb-963 | 103 | P.5 |
| Comb-964 | 104 | P.5 |
| Comb-965 | 105 | P.5 |
| Comb-966 | 106 | P.5 |
| Comb-967 | 107 | P.5 |
| Comb-968 | 108 | P.5 |
| Comb-969 | 109 | P.5 |
| Comb-970 | 110 | P.5 |
| Comb-971 | 111 | P.5 |
| Comb-972 | 112 | P.5 |
| Comb-973 | 113 | P.5 |
| Comb-974 | 114 | P.5 |
| Comb-975 | 115 | P.5 |
| Comb-976 | 116 | P.5 |
| Comb-977 | 117 | P.5 |
| Comb-978 | 118 | P.5 |
| Comb-979 | 119 | P.5 |
| Comb-980 | 120 | P.5 |
| Comb-981 | 121 | P.5 |
| Comb-982 | 122 | P.5 |
| Comb-983 | 123 | P.5 |
| Comb-984 | 124 | P.5 |
| Comb-985 | 125 | P.5 |
| Comb-986 | 126 | P.5 |
| Comb-987 | 127 | P.5 |
| Comb-988 | 128 | P.5 |
| Comb-989 | 129 | P.5 |
| Comb-990 | 130 | P.5 |
| Comb-991 | 131 | P.5 |
| Comb-992 | 132 | P.5 |
| Comb-993 | 133 | P.5 |
| Comb-994 | 134 | P.5 |
| Comb-995 | 135 | P.5 |
| Comb-996 | 136 | P.5 |
| Comb-997 | 137 | P.5 |
| Comb-998 | 138 | P.5 |
| Comb-999 | 139 | P.5 |
| Comb-1000 | 140 | P.5 |
| Comb-1001 | 141 | P.5 |
| Comb-1002 | 142 | P.5 |
| Comb-1003 | 143 | P.5 |
| Comb-1004 | 144 | P.5 |
| Comb-1005 | 145 | P.5 |
| Comb-1006 | 146 | P.5 |
| Comb-1007 | 147 | P.5 |
| Comb-1008 | 148 | P.5 |
| Comb-1009 | 149 | P.5 |
| Comb-1010 | 150 | P.5 |
| Comb-1011 | 151 | P.5 |
| Comb-1012 | 152 | P.5 |
| Comb-1013 | 153 | P.5 |
| Comb-1014 | 154 | P.5 |
| Comb-1015 | 155 | P.5 |
| Comb-1016 | 156 | P.5 |
| Comb-1017 | 157 | P.5 |
| Comb-1018 | 158 | P.5 |
| Comb-1019 | 159 | P.5 |
| Comb-1020 | 160 | P.5 |
| Comb-1021 | 161 | P.5 |
| Comb-1022 | 162 | P.5 |
| Comb-1023 | 163 | P.5 |
| Comb-1024 | 164 | P.5 |
| Comb-1025 | 165 | P.5 |
| Comb-1026 | 166 | P.5 |
| Comb-1027 | 167 | P.5 |
| Comb-1028 | 168 | P.5 |
| Comb-1029 | 169 | P.5 |
| Comb-1030 | 170 | P.5 |
| Comb-1031 | 171 | P.5 |
| Comb-1032 | 172 | P.5 |
| Comb-1033 | 173 | P.5 |
| Comb-1034 | 174 | P.5 |
| Comb-1035 | 175 | P.5 |
| Comb-1036 | 176 | P.5 |
| Comb-1037 | 177 | P.5 |
| Comb-1038 | 178 | P.5 |
| Comb-1039 | 179 | P.5 |
| Comb-1040 | 180 | P.5 |
| Comb-1041 | 181 | P.5 |
| Comb-1042 | 182 | P.5 |
| Comb-1043 | 183 | P.5 |
| Comb-1044 | 184 | P.5 |
| Comb-1045 | 185 | P.5 |
| Comb-1046 | 186 | P.5 |
| Comb-1047 | 187 | P.5 |
| Comb-1048 | 188 | P.5 |
| Comb-1049 | 189 | P.5 |
| Comb-1050 | 190 | P.5 |
| Comb-1051 | 191 | P.5 |
| Comb-1052 | 192 | P.5 |
| Comb-1053 | 193 | P.5 |
| Comb-1054 | 194 | P.5 |
| Comb-1055 | 195 | P.5 |
| Comb-1056 | 196 | P.5 |
| Comb-1057 | 197 | P.5 |
| Comb-1058 | 198 | P.5 |
| Comb-1059 | 199 | P.5 |
| Comb-1060 | 200 | P.5 |
| Comb-1061 | 201 | P.5 |
| Comb-1062 | 202 | P.5 |
| Comb-1063 | 203 | P.5 |
| Comb-1064 | 204 | P.5 |
| Comb-1065 | 205 | P.5 |
| Comb-1066 | 206 | P.5 |
| Comb-1067 | 207 | P.5 |
| Comb-1068 | 208 | P.5 |
| Comb-1069 | 209 | P.5 |
| Comb-1070 | 210 | P.5 |
| Comb-1071 | 211 | P.5 |
| Comb-1072 | 212 | P.5 |
| Comb-1073 | 213 | P.5 |
| Comb-1074 | 214 | P.5 |
| Comb-1075 | 215 | P.5 |
| Comb-1076 | 1 | P.6 |
| Comb-1077 | 2 | P.6 |
| Comb-1078 | 3 | P.6 |
| Comb-1079 | 4 | P.6 |
| Comb-1080 | 5 | P.6 |
| Comb-1081 | 6 | P.6 |
| Comb-1082 | 7 | P.6 |
| Comb-1083 | 8 | P.6 |
| Comb-1084 | 9 | P.6 |
| Comb-1085 | 10 | P.6 |
| Comb-1086 | 11 | P.6 |
| Comb-1087 | 12 | P.6 |
| Comb-1088 | 13 | P.6 |
| Comb-1089 | 14 | P.6 |
| Comb-1090 | 15 | P.6 |
| Comb-1091 | 16 | P.6 |
| Comb-1092 | 17 | P.6 |
| Comb-1093 | 18 | P.6 |
| Comb-1094 | 19 | P.6 |
| Comb-1095 | 20 | P.6 |
| Comb-1096 | 21 | P.6 |
| Comb-1097 | 22 | P.6 |
| Comb-1098 | 23 | P.6 |
| Comb-1099 | 24 | P.6 |
| Comb-1100 | 25 | P.6 |
| Comb-1101 | 26 | P.6 |
| Comb-1102 | 27 | P.6 |
| Comb-1103 | 28 | P.6 |
| Comb-1104 | 29 | P.6 |
| Comb-1105 | 30 | P.6 |
| Comb-1106 | 31 | P.6 |
| Comb-1107 | 32 | P.6 |
| Comb-1108 | 33 | P.6 |
| Comb-1109 | 34 | P.6 |
| Comb-1110 | 35 | P.6 |
| Comb-1111 | 36 | P.6 |
| Comb-1112 | 37 | P.6 |
| Comb-1113 | 38 | P.6 |
| Comb-1114 | 39 | P.6 |
| Comb-1115 | 40 | P.6 |
| Comb-1116 | 41 | P.6 |
| Comb-1117 | 42 | P.6 |
| Comb-1118 | 43 | P.6 |
| Comb-1119 | 44 | P.6 |
| Comb-1120 | 45 | P.6 |
| Comb-1121 | 46 | P.6 |
| Comb-1122 | 47 | P.6 |
| Comb-1123 | 48 | P.6 |
| Comb-1124 | 49 | P.6 |
| Comb-1125 | 50 | P.6 |
| Comb-1126 | 51 | P.6 |
| Comb-1127 | 52 | P.6 |
| Comb-1128 | 53 | P.6 |
| Comb-1129 | 54 | P.6 |
| Comb-1130 | 55 | P.6 |
| Comb-1131 | 56 | P.6 |
| Comb-1132 | 57 | P.6 |
| Comb-1133 | 58 | P.6 |
| Comb-1134 | 59 | P.6 |
| Comb-1135 | 60 | P.6 |
| Comb-1136 | 61 | P.6 |
| Comb-1137 | 62 | P.6 |
| Comb-1138 | 63 | P.6 |
| Comb-1139 | 64 | P.6 |
| Comb-1140 | 65 | P.6 |
| Comb-1141 | 66 | P.6 |
| Comb-1142 | 67 | P.6 |
| Comb-1143 | 68 | P.6 |
| Comb-1144 | 69 | P.6 |
| Comb-1145 | 70 | P.6 |
| Comb-1146 | 71 | P.6 |
| Comb-1147 | 72 | P.6 |
| Comb-1148 | 73 | P.6 |
| Comb-1149 | 74 | P.6 |
| Comb-1150 | 75 | P.6 |
| Comb-1151 | 76 | P.6 |
| Comb-1152 | 77 | P.6 |
| Comb-1153 | 78 | P.6 |
| Comb-1154 | 79 | P.6 |
| Comb-1155 | 80 | P.6 |
| Comb-1156 | 81 | P.6 |
| Comb-1157 | 82 | P.6 |
| Comb-1158 | 83 | P.6 |
| Comb-1159 | 84 | P.6 |
| Comb-1160 | 85 | P.6 |
| Comb-1161 | 86 | P.6 |
| Comb-1162 | 87 | P.6 |
| Comb-1163 | 88 | P.6 |
| Comb-1164 | 89 | P.6 |
| Comb-1165 | 90 | P.6 |
| Comb-1166 | 91 | P.6 |
| Comb-1167 | 92 | P.6 |
| Comb-1168 | 93 | P.6 |
| Comb-1169 | 94 | P.6 |
| Comb-1170 | 95 | P.6 |
| Comb-1171 | 96 | P.6 |
| Comb-1172 | 97 | P.6 |
| Comb-1173 | 98 | P.6 |
| Comb-1174 | 99 | P.6 |
| Comb-1175 | 100 | P.6 |
| Comb-1176 | 101 | P.6 |
| Comb-1177 | 102 | P.6 |
| Comb-1178 | 103 | P.6 |
| Comb-1179 | 104 | P.6 |
| Comb-1180 | 105 | P.6 |
| Comb-1181 | 106 | P.6 |
| Comb-1182 | 107 | P.6 |
| Comb-1183 | 108 | P.6 |
| Comb-1184 | 109 | P.6 |
| Comb-1185 | 110 | P.6 |
| Comb-1186 | 111 | P.6 |
| Comb-1187 | 112 | P.6 |
| Comb-1188 | 113 | P.6 |
| Comb-1189 | 114 | P.6 |
| Comb-1190 | 115 | P.6 |
| Comb-1191 | 116 | P.6 |
| Comb-1192 | 117 | P.6 |
| Comb-1193 | 118 | P.6 |
| Comb-1194 | 119 | P.6 |
| Comb-1195 | 120 | P.6 |
| Comb-1196 | 121 | P.6 |
| Comb-1197 | 122 | P.6 |
| Comb-1198 | 123 | P.6 |
| Comb-1199 | 124 | P.6 |
| Comb-1200 | 125 | P.6 |
| Comb-1201 | 126 | P.6 |
| Comb-1202 | 127 | P.6 |
| Comb-1203 | 128 | P.6 |
| Comb-1204 | 129 | P.6 |
| Comb-1205 | 130 | P.6 |
| Comb-1206 | 131 | P.6 |
| Comb-1207 | 132 | P.6 |
| Comb-1208 | 133 | P.6 |
| Comb-1209 | 134 | P.6 |
| Comb-1210 | 135 | P.6 |
| Comb-1211 | 136 | P.6 |
| Comb-1212 | 137 | P.6 |
| Comb-1213 | 138 | P.6 |
| Comb-1214 | 139 | P.6 |
| Comb-1215 | 140 | P.6 |
| Comb-1216 | 141 | P.6 |
| Comb-1217 | 142 | P.6 |
| Comb-1218 | 143 | P.6 |
| Comb-1219 | 144 | P.6 |
| Comb-1220 | 145 | P.6 |
| Comb-1221 | 146 | P.6 |
| Comb-1222 | 147 | P.6 |
| Comb-1223 | 148 | P.6 |
| Comb-1224 | 149 | P.6 |
| Comb-1225 | 150 | P.6 |
| Comb-1226 | 151 | P.6 |
| Comb-1227 | 152 | P.6 |
| Comb-1228 | 153 | P.6 |
| Comb-1229 | 154 | P.6 |
| Comb-1230 | 155 | P.6 |
| Comb-1231 | 156 | P.6 |
| Comb-1232 | 157 | P.6 |
| Comb-1233 | 158 | P.6 |
| Comb-1234 | 159 | P.6 |
| Comb-1235 | 160 | P.6 |
| Comb-1236 | 161 | P.6 |
| Comb-1237 | 162 | P.6 |
| Comb-1238 | 163 | P.6 |
| Comb-1239 | 164 | P.6 |
| Comb-1240 | 165 | P.6 |
| Comb-1241 | 166 | P.6 |
| Comb-1242 | 167 | P.6 |
| Comb-1243 | 168 | P.6 |
| Comb-1244 | 169 | P.6 |
| Comb-1245 | 170 | P.6 |
| Comb-1246 | 171 | P.6 |
| Comb-1247 | 172 | P.6 |
| Comb-1248 | 173 | P.6 |
| Comb-1249 | 174 | P.6 |
| Comb-1250 | 175 | P.6 |
| Comb-1251 | 176 | P.6 |
| Comb-1252 | 177 | P.6 |
| Comb-1253 | 178 | P.6 |
| Comb-1254 | 179 | P.6 |
| Comb-1255 | 180 | P.6 |
| Comb-1256 | 181 | P.6 |
| Comb-1257 | 182 | P.6 |
| Comb-1258 | 183 | P.6 |
| Comb-1259 | 184 | P.6 |
| Comb-1260 | 185 | P.6 |
| Comb-1261 | 186 | P.6 |
| Comb-1262 | 187 | P.6 |
| Comb-1263 | 188 | P.6 |
| Comb-1264 | 189 | P.6 |
| Comb-1265 | 190 | P.6 |
| Comb-1266 | 191 | P.6 |
| Comb-1267 | 192 | P.6 |
| Comb-1268 | 193 | P.6 |
| Comb-1269 | 194 | P.6 |
| Comb-1270 | 195 | P.6 |
| Comb-1271 | 196 | P.6 |
| Comb-1272 | 197 | P.6 |
| Comb-1273 | 198 | P.6 |
| Comb-1274 | 199 | P.6 |
| Comb-1275 | 200 | P.6 |
| Comb-1276 | 201 | P.6 |
| Comb-1277 | 202 | P.6 |
| Comb-1278 | 203 | P.6 |
| Comb-1279 | 204 | P.6 |
| Comb-1280 | 205 | P.6 |
| Comb-1281 | 206 | P.6 |
| Comb-1282 | 207 | P.6 |
| Comb-1283 | 208 | P.6 |
| Comb-1284 | 209 | P.6 |
| Comb-1285 | 210 | P.6 |
| Comb-1286 | 211 | P.6 |
| Comb-1287 | 212 | P.6 |
| Comb-1288 | 213 | P.6 |
| Comb-1289 | 214 | P.6 |
| Comb-1290 | 215 | P.6 |
| Comb-1291 | 1 | P.7 |
| Comb-1292 | 2 | P.7 |
| Comb-1293 | 3 | P.7 |
| Comb-1294 | 4 | P.7 |
| Comb-1295 | 5 | P.7 |
| Comb-1296 | 6 | P.7 |
| Comb-1297 | 7 | P.7 |
| Comb-1298 | 8 | P.7 |
| Comb-1299 | 9 | P.7 |
| Comb-1300 | 10 | P.7 |
| Comb-1301 | 11 | P.7 |
| Comb-1302 | 12 | P.7 |
| Comb-1303 | 13 | P.7 |
| Comb-1304 | 14 | P.7 |
| Comb-1305 | 15 | P.7 |
| Comb-1306 | 16 | P.7 |
| Comb-1307 | 17 | P.7 |
| Comb-1308 | 18 | P.7 |
| Comb-1309 | 19 | P.7 |
| Comb-1310 | 20 | P.7 |
| Comb-1311 | 21 | P.7 |
| Comb-1312 | 22 | P.7 |
| Comb-1313 | 23 | P.7 |
| Comb-1314 | 24 | P.7 |
| Comb-1315 | 25 | P.7 |
| Comb-1316 | 26 | P.7 |
| Comb-1317 | 27 | P.7 |
| Comb-1318 | 28 | P.7 |
| Comb-1319 | 29 | P.7 |
| Comb-1320 | 30 | P.7 |
| Comb-1321 | 31 | P.7 |
| Comb-1322 | 32 | P.7 |
| Comb-1323 | 33 | P.7 |
| Comb-1324 | 34 | P.7 |
| Comb-1325 | 35 | P.7 |
| Comb-1326 | 36 | P.7 |
| Comb-1327 | 37 | P.7 |
| Comb-1328 | 38 | P.7 |
| Comb-1329 | 39 | P.7 |
| Comb-1330 | 40 | P.7 |
| Comb-1331 | 41 | P.7 |
| Comb-1332 | 42 | P.7 |
| Comb-1333 | 43 | P.7 |
| Comb-1334 | 44 | P.7 |
| Comb-1335 | 45 | P.7 |
| Comb-1336 | 46 | P.7 |
| Comb-1337 | 47 | P.7 |
| Comb-1338 | 48 | P.7 |
| Comb-1339 | 49 | P.7 |
| Comb-1340 | 50 | P.7 |
| Comb-1341 | 51 | P.7 |
| Comb-1342 | 52 | P.7 |
| Comb-1343 | 53 | P.7 |
| Comb-1344 | 54 | P.7 |
| Comb-1345 | 55 | P.7 |
| Comb-1346 | 56 | P.7 |
| Comb-1347 | 57 | P.7 |
| Comb-1348 | 58 | P.7 |
| Comb-1349 | 59 | P.7 |
| Comb-1350 | 60 | P.7 |
| Comb-1351 | 61 | P.7 |
| Comb-1352 | 62 | P.7 |
| Comb-1353 | 63 | P.7 |
| Comb-1354 | 64 | P.7 |
| Comb-1355 | 65 | P.7 |
| Comb-1356 | 66 | P.7 |
| Comb-1357 | 67 | P.7 |
| Comb-1358 | 68 | P.7 |
| Comb-1359 | 69 | P.7 |
| Comb-1360 | 70 | P.7 |
| Comb-1361 | 71 | P.7 |
| Comb-1362 | 72 | P.7 |
| Comb-1363 | 73 | P.7 |
| Comb-1364 | 74 | P.7 |
| Comb-1365 | 75 | P.7 |
| Comb-1366 | 76 | P.7 |
| Comb-1367 | 77 | P.7 |
| Comb-1368 | 78 | P.7 |
| Comb-1369 | 79 | P.7 |
| Comb-1370 | 80 | P.7 |
| Comb-1371 | 81 | P.7 |
| Comb-1372 | 82 | P.7 |
| Comb-1373 | 83 | P.7 |
| Comb-1374 | 84 | P.7 |
| Comb-1375 | 85 | P.7 |
| Comb-1376 | 86 | P.7 |
| Comb-1377 | 87 | P.7 |
| Comb-1378 | 88 | P.7 |
| Comb-1379 | 89 | P.7 |
| Comb-1380 | 90 | P.7 |
| Comb-1381 | 91 | P.7 |
| Comb-1382 | 92 | P.7 |
| Comb-1383 | 93 | P.7 |
| Comb-1384 | 94 | P.7 |
| Comb-1385 | 95 | P.7 |
| Comb-1386 | 96 | P.7 |
| Comb-1387 | 97 | P.7 |
| Comb-1388 | 98 | P.7 |
| Comb-1389 | 99 | P.7 |
| Comb-1390 | 100 | P.7 |
| Comb-1391 | 101 | P.7 |
| Comb-1392 | 102 | P.7 |
| Comb-1393 | 103 | P.7 |
| Comb-1394 | 104 | P.7 |
| Comb-1395 | 105 | P.7 |
| Comb-1396 | 106 | P.7 |
| Comb-1397 | 107 | P.7 |
| Comb-1398 | 108 | P.7 |
| Comb-1399 | 109 | P.7 |
| Comb-1400 | 110 | P.7 |
| Comb-1401 | 111 | P.7 |
| Comb-1402 | 112 | P.7 |
| Comb-1403 | 113 | P.7 |
| Comb-1404 | 114 | P.7 |
| Comb-1405 | 115 | P.7 |
| Comb-1406 | 116 | P.7 |
| Comb-1407 | 117 | P.7 |
| Comb-1408 | 118 | P.7 |
| Comb-1409 | 119 | P.7 |
| Comb-1410 | 120 | P.7 |
| Comb-1411 | 121 | P.7 |
| Comb-1412 | 122 | P.7 |
| Comb-1413 | 123 | P.7 |
| Comb-1414 | 124 | P.7 |
| Comb-1415 | 125 | P.7 |
| Comb-1416 | 126 | P.7 |
| Comb-1417 | 127 | P.7 |
| Comb-1418 | 128 | P.7 |
| Comb-1419 | 129 | P.7 |
| Comb-1420 | 130 | P.7 |
| Comb-1421 | 131 | P.7 |
| Comb-1422 | 132 | P.7 |
| Comb-1423 | 133 | P.7 |
| Comb-1424 | 134 | P.7 |
| Comb-1425 | 135 | P.7 |
| Comb-1426 | 136 | P.7 |
| Comb-1427 | 137 | P.7 |
| Comb-1428 | 138 | P.7 |
| Comb-1429 | 139 | P.7 |
| Comb-1430 | 140 | P.7 |
| Comb-1431 | 141 | P.7 |
| Comb-1432 | 142 | P.7 |
| Comb-1433 | 143 | P.7 |
| Comb-1434 | 144 | P.7 |
| Comb-1435 | 145 | P.7 |
| Comb-1436 | 146 | P.7 |
| Comb-1437 | 147 | P.7 |
| Comb-1438 | 148 | P.7 |
| Comb-1439 | 149 | P.7 |
| Comb-1440 | 150 | P.7 |
| Comb-1441 | 151 | P.7 |
| Comb-1442 | 152 | P.7 |
| Comb-1443 | 153 | P.7 |
| Comb-1444 | 154 | P.7 |
| Comb-1445 | 155 | P.7 |
| Comb-1446 | 156 | P.7 |
| Comb-1447 | 157 | P.7 |
| Comb-1448 | 158 | P.7 |
| Comb-1449 | 159 | P.7 |
| Comb-1450 | 160 | P.7 |
| Comb-1451 | 161 | P.7 |
| Comb-1452 | 162 | P.7 |
| Comb-1453 | 163 | P.7 |
| Comb-1454 | 164 | P.7 |
| Comb-1455 | 165 | P.7 |
| Comb-1456 | 166 | P.7 |
| Comb-1457 | 167 | P.7 |
| Comb-1458 | 168 | P.7 |
| Comb-1459 | 169 | P.7 |
| Comb-1460 | 170 | P.7 |
| Comb-1461 | 171 | P.7 |
| Comb-1462 | 172 | P.7 |
| Comb-1463 | 173 | P.7 |
| Comb-1464 | 174 | P.7 |
| Comb-1465 | 175 | P.7 |
| Comb-1466 | 176 | P.7 |
| Comb-1467 | 177 | P.7 |
| Comb-1468 | 178 | P.7 |
| Comb-1469 | 179 | P.7 |
| Comb-1470 | 180 | P.7 |
| Comb-1471 | 181 | P.7 |
| Comb-1472 | 182 | P.7 |
| Comb-1473 | 183 | P.7 |
| Comb-1474 | 184 | P.7 |
| Comb-1475 | 185 | P.7 |
| Comb-1476 | 186 | P.7 |
| Comb-1477 | 187 | P.7 |
| Comb-1478 | 188 | P.7 |
| Comb-1479 | 189 | P.7 |
| Comb-1480 | 190 | P.7 |
| Comb-1481 | 191 | P.7 |
| Comb-1482 | 192 | P.7 |
| Comb-1483 | 193 | P.7 |
| Comb-1484 | 194 | P.7 |
| Comb-1485 | 195 | P.7 |
| Comb-1486 | 196 | P.7 |
| Comb-1487 | 197 | P.7 |
| Comb-1488 | 198 | P.7 |
| Comb-1489 | 199 | P.7 |
| Comb-1490 | 200 | P.7 |
| Comb-1491 | 201 | P.7 |
| Comb-1492 | 202 | P.7 |
| Comb-1493 | 203 | P.7 |
| Comb-1494 | 204 | P.7 |
| Comb-1495 | 205 | P.7 |
| Comb-1496 | 206 | P.7 |
| Comb-1497 | 207 | P.7 |
| Comb-1498 | 208 | P.7 |
| Comb-1499 | 209 | P.7 |
| Comb-1500 | 210 | P.7 |
| Comb-1501 | 211 | P.7 |
| Comb-1502 | 212 | P.7 |
| Comb-1503 | 213 | P.7 |
| Comb-1504 | 214 | P.7 |
| Comb-1505 | 215 | P.7 |
| Comb-1506 | 1 | P.8 |
| Comb-1507 | 2 | P.8 |
| Comb-1508 | 3 | P.8 |
| Comb-1509 | 4 | P.8 |
| Comb-1510 | 5 | P.8 |
| Comb-1511 | 6 | P.8 |
| Comb-1512 | 7 | P.8 |
| Comb-1513 | 8 | P.8 |
| Comb-1514 | 9 | P.8 |
| Comb-1515 | 10 | P.8 |
| Comb-1516 | 11 | P.8 |
| Comb-1517 | 12 | P.8 |
| Comb-1518 | 13 | P.8 |
| Comb-1519 | 14 | P.8 |
| Comb-1520 | 15 | P.8 |
| Comb-1521 | 16 | P.8 |
| Comb-1522 | 17 | P.8 |
| Comb-1523 | 18 | P.8 |
| Comb-1524 | 19 | P.8 |
| Comb-1525 | 20 | P.8 |
| Comb-1526 | 21 | P.8 |
| Comb-1527 | 22 | P.8 |
| Comb-1528 | 23 | P.8 |
| Comb-1529 | 24 | P.8 |
| Comb-1530 | 25 | P.8 |
| Comb-1531 | 26 | P.8 |
| Comb-1532 | 27 | P.8 |
| Comb-1533 | 28 | P.8 |
| Comb-1534 | 29 | P.8 |
| Comb-1535 | 30 | P.8 |
| Comb-1536 | 31 | P.8 |
| Comb-1537 | 32 | P.8 |
| Comb-1538 | 33 | P.8 |
| Comb-1539 | 34 | P.8 |
| Comb-1540 | 35 | P.8 |
| Comb-1541 | 36 | P.8 |
| Comb-1542 | 37 | P.8 |
| Comb-1543 | 38 | P.8 |
| Comb-1544 | 39 | P.8 |
| Comb-1545 | 40 | P.8 |
| Comb-1546 | 41 | P.8 |
| Comb-1547 | 42 | P.8 |
| Comb-1548 | 43 | P.8 |
| Comb-1549 | 44 | P.8 |
| Comb-1550 | 45 | P.8 |
| Comb-1551 | 46 | P.8 |
| Comb-1552 | 47 | P.8 |
| Comb-1553 | 48 | P.8 |
| Comb-1554 | 49 | P.8 |
| Comb-1555 | 50 | P.8 |
| Comb-1556 | 51 | P.8 |
| Comb-1557 | 52 | P.8 |
| Comb-1558 | 53 | P.8 |
| Comb-1559 | 54 | P.8 |
| Comb-1560 | 55 | P.8 |
| Comb-1561 | 56 | P.8 |
| Comb-1562 | 57 | P.8 |
| Comb-1563 | 58 | P.8 |
| Comb-1564 | 59 | P.8 |
| Comb-1565 | 60 | P.8 |
| Comb-1566 | 61 | P.8 |
| Comb-1567 | 62 | P.8 |
| Comb-1568 | 63 | P.8 |
| Comb-1569 | 64 | P.8 |
| Comb-1570 | 65 | P.8 |
| Comb-1571 | 66 | P.8 |
| Comb-1572 | 67 | P.8 |
| Comb-1573 | 68 | P.8 |
| Comb-1574 | 69 | P.8 |
| Comb-1575 | 70 | P.8 |
| Comb-1576 | 71 | P.8 |
| Comb-1577 | 72 | P.8 |
| Comb-1578 | 73 | P.8 |
| Comb-1579 | 74 | P.8 |
| Comb-1580 | 75 | P.8 |
| Comb-1581 | 76 | P.8 |
| Comb-1582 | 77 | P.8 |
| Comb-1583 | 78 | P.8 |
| Comb-1584 | 79 | P.8 |
| Comb-1585 | 80 | P.8 |
| Comb-1586 | 81 | P.8 |
| Comb-1587 | 82 | P.8 |
| Comb-1588 | 83 | P.8 |
| Comb-1589 | 84 | P.8 |
| Comb-1590 | 85 | P.8 |
| Comb-1591 | 86 | P.8 |
| Comb-1592 | 87 | P.8 |
| Comb-1593 | 88 | P.8 |
| Comb-1594 | 89 | P.8 |
| Comb-1595 | 90 | P.8 |
| Comb-1596 | 91 | P.8 |
| Comb-1597 | 92 | P.8 |
| Comb-1598 | 93 | P.8 |
| Comb-1599 | 94 | P.8 |
| Comb-1600 | 95 | P.8 |
| Comb-1601 | 96 | P.8 |
| Comb-1602 | 97 | P.8 |
| Comb-1603 | 98 | P.8 |
| Comb-1604 | 99 | P.8 |
| Comb-1605 | 100 | P.8 |
| Comb-1606 | 101 | P.8 |
| Comb-1607 | 102 | P.8 |
| Comb-1608 | 103 | P.8 |
| Comb-1609 | 104 | P.8 |
| Comb-1610 | 105 | P.8 |
| Comb-1611 | 106 | P.8 |
| Comb-1612 | 107 | P.8 |
| Comb-1613 | 108 | P.8 |
| Comb-1614 | 109 | P.8 |
| Comb-1615 | 110 | P.8 |
| Comb-1616 | 111 | P.8 |
| Comb-1617 | 112 | P.8 |
| Comb-1618 | 113 | P.8 |
| Comb-1619 | 114 | P.8 |
| Comb-1620 | 115 | P.8 |
| Comb-1621 | 116 | P.8 |
| Comb-1622 | 117 | P.8 |
| Comb-1623 | 118 | P.8 |
| Comb-1624 | 119 | P.8 |
| Comb-1625 | 120 | P.8 |
| Comb-1626 | 121 | P.8 |
| Comb-1627 | 122 | P.8 |
| Comb-1628 | 123 | P.8 |
| Comb-1629 | 124 | P.8 |
| Comb-1630 | 125 | P.8 |
| Comb-1631 | 126 | P.8 |
| Comb-1632 | 127 | P.8 |
| Comb-1633 | 128 | P.8 |
| Comb-1634 | 129 | P.8 |
| Comb-1635 | 130 | P.8 |
| Comb-1636 | 131 | P.8 |
| Comb-1637 | 132 | P.8 |
| Comb-1638 | 133 | P.8 |
| Comb-1639 | 134 | P.8 |
| Comb-1640 | 135 | P.8 |
| Comb-1641 | 136 | P.8 |
| Comb-1642 | 137 | P.8 |
| Comb-1643 | 138 | P.8 |
| Comb-1644 | 139 | P.8 |
| Comb-1645 | 140 | P.8 |
| Comb-1646 | 141 | P.8 |
| Comb-1647 | 142 | P.8 |
| Comb-1648 | 143 | P.8 |
| Comb-1649 | 144 | P.8 |
| Comb-1650 | 145 | P.8 |
| Comb-1651 | 146 | P.8 |
| Comb-1652 | 147 | P.8 |
| Comb-1653 | 148 | P.8 |
| Comb-1654 | 149 | P.8 |
| Comb-1655 | 150 | P.8 |
| Comb-1656 | 151 | P.8 |
| Comb-1657 | 152 | P.8 |
| Comb-1658 | 153 | P.8 |
| Comb-1659 | 154 | P.8 |
| Comb-1660 | 155 | P.8 |
| Comb-1661 | 156 | P.8 |
| Comb-1662 | 157 | P.8 |
| Comb-1663 | 158 | P.8 |
| Comb-1664 | 159 | P.8 |
| Comb-1665 | 160 | P.8 |
| Comb-1666 | 161 | P.8 |
| Comb-1667 | 162 | P.8 |
| Comb-1668 | 163 | P.8 |
| Comb-1669 | 164 | P.8 |
| Comb-1670 | 165 | P.8 |
| Comb-1671 | 166 | P.8 |
| Comb-1672 | 167 | P.8 |
| Comb-1673 | 168 | P.8 |
| Comb-1674 | 169 | P.8 |
| Comb-1675 | 170 | P.8 |
| Comb-1676 | 171 | P.8 |
| Comb-1677 | 172 | P.8 |
| Comb-1678 | 173 | P.8 |
| Comb-1679 | 174 | P.8 |
| Comb-1680 | 175 | P.8 |
| Comb-1681 | 176 | P.8 |
| Comb-1682 | 177 | P.8 |
| Comb-1683 | 178 | P.8 |
| Comb-1684 | 179 | P.8 |
| Comb-1685 | 180 | P.8 |
| Comb-1686 | 181 | P.8 |
| Comb-1687 | 182 | P.8 |
| Comb-1688 | 183 | P.8 |
| Comb-1689 | 184 | P.8 |
| Comb-1690 | 185 | P.8 |
| Comb-1691 | 186 | P.8 |
| Comb-1692 | 187 | P.8 |
| Comb-1693 | 188 | P.8 |
| Comb-1694 | 189 | P.8 |
| Comb-1695 | 190 | P.8 |
| Comb-1696 | 191 | P.8 |
| Comb-1697 | 192 | P.8 |
| Comb-1698 | 193 | P.8 |
| Comb-1699 | 194 | P.8 |
| Comb-1700 | 195 | P.8 |
| Comb-1701 | 196 | P.8 |
| Comb-1702 | 197 | P.8 |
| Comb-1703 | 198 | P.8 |
| Comb-1704 | 199 | P.8 |
| Comb-1705 | 200 | P.8 |
| Comb-1706 | 201 | P.8 |
| Comb-1707 | 202 | P.8 |
| Comb-1708 | 203 | P.8 |
| Comb-1709 | 204 | P.8 |
| Comb-1710 | 205 | P.8 |
| Comb-1711 | 206 | P.8 |
| Comb-1712 | 207 | P.8 |
| Comb-1713 | 208 | P.8 |
| Comb-1714 | 209 | P.8 |
| Comb-1715 | 210 | P.8 |
| Comb-1716 | 211 | P.8 |
| Comb-1717 | 212 | P.8 |
| Comb-1718 | 213 | P.8 |
| Comb-1719 | 214 | P.8 |
| Comb-1720 | 215 | P.8 |
| Comb-1721 | 1 | P.9 |
| Comb-1722 | 2 | P.9 |
| Comb-1723 | 3 | P.9 |
| Comb-1724 | 4 | P.9 |
| Comb-1725 | 5 | P.9 |
| Comb-1726 | 6 | P.9 |
| Comb-1727 | 7 | P.9 |
| Comb-1728 | 8 | P.9 |
| Comb-1729 | 9 | P.9 |
| Comb-1730 | 10 | P.9 |
| Comb-1731 | 11 | P.9 |
| Comb-1732 | 12 | P.9 |
| Comb-1733 | 13 | P.9 |
| Comb-1734 | 14 | P.9 |
| Comb-1735 | 15 | P.9 |
| Comb-1736 | 16 | P.9 |
| Comb-1737 | 17 | P.9 |
| Comb-1738 | 18 | P.9 |
| Comb-1739 | 19 | P.9 |
| Comb-1740 | 20 | P.9 |
| Comb-1741 | 21 | P.9 |
| Comb-1742 | 22 | P.9 |
| Comb-1743 | 23 | P.9 |
| Comb-1744 | 24 | P.9 |
| Comb-1745 | 25 | P.9 |
| Comb-1746 | 26 | P.9 |
| Comb-1747 | 27 | P.9 |
| Comb-1748 | 28 | P.9 |
| Comb-1749 | 29 | P.9 |
| Comb-1750 | 30 | P.9 |
| Comb-1751 | 31 | P.9 |
| Comb-1752 | 32 | P.9 |
| Comb-1753 | 33 | P.9 |
| Comb-1754 | 34 | P.9 |
| Comb-1755 | 35 | P.9 |
| Comb-1756 | 36 | P.9 |
| Comb-1757 | 37 | P.9 |
| Comb-1758 | 38 | P.9 |
| Comb-1759 | 39 | P.9 |
| Comb-1760 | 40 | P.9 |
| Comb-1761 | 41 | P.9 |
| Comb-1762 | 42 | P.9 |
| Comb-1763 | 43 | P.9 |
| Comb-1764 | 44 | P.9 |
| Comb-1765 | 45 | P.9 |
| Comb-1766 | 46 | P.9 |
| Comb-1767 | 47 | P.9 |
| Comb-1768 | 48 | P.9 |
| Comb-1769 | 49 | P.9 |
| Comb-1770 | 50 | P.9 |
| Comb-1771 | 51 | P.9 |
| Comb-1772 | 52 | P.9 |
| Comb-1773 | 53 | P.9 |
| Comb-1774 | 54 | P.9 |
| Comb-1775 | 55 | P.9 |
| Comb-1776 | 56 | P.9 |
| Comb-1777 | 57 | P.9 |
| Comb-1778 | 58 | P.9 |
| Comb-1779 | 59 | P.9 |
| Comb-1780 | 60 | P.9 |
| Comb-1781 | 61 | P.9 |
| Comb-1782 | 62 | P.9 |
| Comb-1783 | 63 | P.9 |
| Comb-1784 | 64 | P.9 |
| Comb-1785 | 65 | P.9 |
| Comb-1786 | 66 | P.9 |
| Comb-1787 | 67 | P.9 |
| Comb-1788 | 68 | P.9 |
| Comb-1789 | 69 | P.9 |
| Comb-1790 | 70 | P.9 |
| Comb-1791 | 71 | P.9 |
| Comb-1792 | 72 | P.9 |
| Comb-1793 | 73 | P.9 |
| Comb-1794 | 74 | P.9 |
| Comb-1795 | 75 | P.9 |
| Comb-1796 | 76 | P.9 |
| Comb-1797 | 77 | P.9 |
| Comb-1798 | 78 | P.9 |
| Comb-1799 | 79 | P.9 |
| Comb-1800 | 80 | P.9 |
| Comb-1801 | 81 | P.9 |
| Comb-1802 | 82 | P.9 |
| Comb-1803 | 83 | P.9 |
| Comb-1804 | 84 | P.9 |
| Comb-1805 | 85 | P.9 |
| Comb-1806 | 86 | P.9 |
| Comb-1807 | 87 | P.9 |
| Comb-1808 | 88 | P.9 |
| Comb-1809 | 89 | P.9 |
| Comb-1810 | 90 | P.9 |
| Comb-1811 | 91 | P.9 |
| Comb-1812 | 92 | P.9 |
| Comb-1813 | 93 | P.9 |
| Comb-1814 | 94 | P.9 |
| Comb-1815 | 95 | P.9 |
| Comb-1816 | 96 | P.9 |
| Comb-1817 | 97 | P.9 |
| Comb-1818 | 98 | P.9 |
| Comb-1819 | 99 | P.9 |
| Comb-1820 | 100 | P.9 |
| Comb-1821 | 101 | P.9 |
| Comb-1822 | 102 | P.9 |
| Comb-1823 | 103 | P.9 |
| Comb-1824 | 104 | P.9 |
| Comb-1825 | 105 | P.9 |
| Comb-1826 | 106 | P.9 |
| Comb-1827 | 107 | P.9 |
| Comb-1828 | 108 | P.9 |
| Comb-1829 | 109 | P.9 |
| Comb-1830 | 110 | P.9 |
| Comb-1831 | 111 | P.9 |
| Comb-1832 | 112 | P.9 |
| Comb-1833 | 113 | P.9 |
| Comb-1834 | 114 | P.9 |
| Comb-1835 | 115 | P.9 |
| Comb-1836 | 116 | P.9 |
| Comb-1837 | 117 | P.9 |
| Comb-1838 | 118 | P.9 |
| Comb-1839 | 119 | P.9 |
| Comb-1840 | 120 | P.9 |
| Comb-1841 | 121 | P.9 |
| Comb-1842 | 122 | P.9 |
| Comb-1843 | 123 | P.9 |
| Comb-1844 | 124 | P.9 |
| Comb-1845 | 125 | P.9 |
| Comb-1846 | 126 | P.9 |
| Comb-1847 | 127 | P.9 |
| Comb-1848 | 128 | P.9 |
| Comb-1849 | 129 | P.9 |
| Comb-1850 | 130 | P.9 |
| Comb-1851 | 131 | P.9 |
| Comb-1852 | 132 | P.9 |
| Comb-1853 | 133 | P.9 |
| Comb-1854 | 134 | P.9 |
| Comb-1855 | 135 | P.9 |
| Comb-1856 | 136 | P.9 |
| Comb-1857 | 137 | P.9 |
| Comb-1858 | 138 | P.9 |
| Comb-1859 | 139 | P.9 |
| Comb-1860 | 140 | P.9 |
| Comb-1861 | 141 | P.9 |
| Comb-1862 | 142 | P.9 |
| Comb-1863 | 143 | P.9 |
| Comb-1864 | 144 | P.9 |
| Comb-1865 | 145 | P.9 |
| Comb-1866 | 146 | P.9 |
| Comb-1867 | 147 | P.9 |
| Comb-1868 | 148 | P.9 |
| Comb-1869 | 149 | P.9 |
| Comb-1870 | 150 | P.9 |
| Comb-1871 | 151 | P.9 |
| Comb-1872 | 152 | P.9 |
| Comb-1873 | 153 | P.9 |
| Comb-1874 | 154 | P.9 |
| Comb-1875 | 155 | P.9 |
| Comb-1876 | 156 | P.9 |
| Comb-1877 | 157 | P.9 |
| Comb-1878 | 158 | P.9 |
| Comb-1879 | 159 | P.9 |
| Comb-1880 | 160 | P.9 |
| Comb-1881 | 161 | P.9 |
| Comb-1882 | 162 | P.9 |
| Comb-1883 | 163 | P.9 |
| Comb-1884 | 164 | P.9 |
| Comb-1885 | 165 | P.9 |
| Comb-1886 | 166 | P.9 |
| Comb-1887 | 167 | P.9 |
| Comb-1888 | 168 | P.9 |
| Comb-1889 | 169 | P.9 |
| Comb-1890 | 170 | P.9 |
| Comb-1891 | 171 | P.9 |
| Comb-1892 | 172 | P.9 |
| Comb-1893 | 173 | P.9 |
| Comb-1894 | 174 | P.9 |
| Comb-1895 | 175 | P.9 |
| Comb-1896 | 176 | P.9 |
| Comb-1897 | 177 | P.9 |
| Comb-1898 | 178 | P.9 |
| Comb-1899 | 179 | P.9 |
| Comb-1900 | 180 | P.9 |
| Comb-1901 | 181 | P.9 |
| Comb-1902 | 182 | P.9 |
| Comb-1903 | 183 | P.9 |
| Comb-1904 | 184 | P.9 |
| Comb-1905 | 185 | P.9 |
| Comb-1906 | 186 | P.9 |
| Comb-1907 | 187 | P.9 |
| Comb-1908 | 188 | P.9 |
| Comb-1909 | 189 | P.9 |
| Comb-1910 | 190 | P.9 |
| Comb-1911 | 191 | P.9 |
| Comb-1912 | 192 | P.9 |
| Comb-1913 | 193 | P.9 |
| Comb-1914 | 194 | P.9 |
| Comb-1915 | 195 | P.9 |
| Comb-1916 | 196 | P.9 |
| Comb-1917 | 197 | P.9 |
| Comb-1918 | 198 | P.9 |
| Comb-1919 | 199 | P.9 |
| Comb-1920 | 200 | P.9 |
| Comb-1921 | 201 | P.9 |
| Comb-1922 | 202 | P.9 |
| Comb-1923 | 203 | P.9 |
| Comb-1924 | 204 | P.9 |
| Comb-1925 | 205 | P.9 |
| Comb-1926 | 206 | P.9 |
| Comb-1927 | 207 | P.9 |
| Comb-1928 | 208 | P.9 |
| Comb-1929 | 209 | P.9 |
| Comb-1930 | 210 | P.9 |
| Comb-1931 | 211 | P.9 |
| Comb-1932 | 212 | P.9 |
| Comb-1933 | 213 | P.9 |
| Comb-1934 | 214 | P.9 |
| Comb-1935 | 215 | P.9 |
| Comb-1936 | 1 | P.10 |
| Comb-1937 | 2 | P.10 |
| Comb-1938 | 3 | P.10 |
| Comb-1939 | 4 | P.10 |
| Comb-1940 | 5 | P.10 |
| Comb-1941 | 6 | P.10 |
| Comb-1942 | 7 | P.10 |
| Comb-1943 | 8 | P.10 |
| Comb-1944 | 9 | P.10 |
| Comb-1945 | 10 | P.10 |
| Comb-1946 | 11 | P.10 |
| Comb-1947 | 12 | P.10 |
| Comb-1948 | 13 | P.10 |
| Comb-1949 | 14 | P.10 |
| Comb-1950 | 15 | P.10 |
| Comb-1951 | 16 | P.10 |
| Comb-1952 | 17 | P.10 |
| Comb-1953 | 18 | P.10 |
| Comb-1954 | 19 | P.10 |
| Comb-1955 | 20 | P.10 |
| Comb-1956 | 21 | P.10 |
| Comb-1957 | 22 | P.10 |
| Comb-1958 | 23 | P.10 |
| Comb-1959 | 24 | P.10 |
| Comb-1960 | 25 | P.10 |
| Comb-1961 | 26 | P.10 |
| Comb-1962 | 27 | P.10 |
| Comb-1963 | 28 | P.10 |
| Comb-1964 | 29 | P.10 |
| Comb-1965 | 30 | P.10 |
| Comb-1966 | 31 | P.10 |
| Comb-1967 | 32 | P.10 |
| Comb-1968 | 33 | P.10 |
| Comb-1969 | 34 | P.10 |
| Comb-1970 | 35 | P.10 |
| Comb-1971 | 36 | P.10 |
| Comb-1972 | 37 | P.10 |
| Comb-1973 | 38 | P.10 |
| Comb-1974 | 39 | P.10 |
| Comb-1975 | 40 | P.10 |
| Comb-1976 | 41 | P.10 |
| Comb-1977 | 42 | P.10 |
| Comb-1978 | 43 | P.10 |
| Comb-1979 | 44 | P.10 |
| Comb-1980 | 45 | P.10 |
| Comb-1981 | 46 | P.10 |
| Comb-1982 | 47 | P.10 |
| Comb-1983 | 48 | P.10 |
| Comb-1984 | 49 | P.10 |
| Comb-1985 | 50 | P.10 |
| Comb-1986 | 51 | P.10 |
| Comb-1987 | 52 | P.10 |
| Comb-1988 | 53 | P.10 |
| Comb-1989 | 54 | P.10 |
| Comb-1990 | 55 | P.10 |
| Comb-1991 | 56 | P.10 |
| Comb-1992 | 57 | P.10 |
| Comb-1993 | 58 | P.10 |
| Comb-1994 | 59 | P.10 |
| Comb-1995 | 60 | P.10 |
| Comb-1996 | 61 | P.10 |
| Comb-1997 | 62 | P.10 |
| Comb-1998 | 63 | P.10 |
| Comb-1999 | 64 | P.10 |
| Comb-2000 | 65 | P.10 |
| Comb-2001 | 66 | P.10 |
| Comb-2002 | 67 | P.10 |
| Comb-2003 | 68 | P.10 |
| Comb-2004 | 69 | P.10 |
| Comb-2005 | 70 | P.10 |
| Comb-2006 | 71 | P.10 |
| Comb-2007 | 72 | P.10 |
| Comb-2008 | 73 | P.10 |
| Comb-2009 | 74 | P.10 |
| Comb-2010 | 75 | P.10 |
| Comb-2011 | 76 | P.10 |
| Comb-2012 | 77 | P.10 |
| Comb-2013 | 78 | P.10 |
| Comb-2014 | 79 | P.10 |
| Comb-2015 | 80 | P.10 |
| Comb-2016 | 81 | P.10 |
| Comb-2017 | 82 | P.10 |
| Comb-2018 | 83 | P.10 |
| Comb-2019 | 84 | P.10 |
| Comb-2020 | 85 | P.10 |
| Comb-2021 | 86 | P.10 |
| Comb-2022 | 87 | P.10 |
| Comb-2023 | 88 | P.10 |
| Comb-2024 | 89 | P.10 |
| Comb-2025 | 90 | P.10 |
| Comb-2026 | 91 | P.10 |
| Comb-2027 | 92 | P.10 |
| Comb-2028 | 93 | P.10 |
| Comb-2029 | 94 | P.10 |
| Comb-2030 | 95 | P.10 |
| Comb-2031 | 96 | P.10 |
| Comb-2032 | 97 | P.10 |
| Comb-2033 | 98 | P.10 |
| Comb-2034 | 99 | P.10 |
| Comb-2035 | 100 | P.10 |
| Comb-2036 | 101 | P.10 |
| Comb-2037 | 102 | P.10 |
| Comb-2038 | 103 | P.10 |
| Comb-2039 | 104 | P.10 |
| Comb-2040 | 105 | P.10 |
| Comb-2041 | 106 | P.10 |
| Comb-2042 | 107 | P.10 |
| Comb-2043 | 108 | P.10 |
| Comb-2044 | 109 | P.10 |
| Comb-2045 | 110 | P.10 |
| Comb-2046 | 111 | P.10 |
| Comb-2047 | 112 | P.10 |
| Comb-2048 | 113 | P.10 |
| Comb-2049 | 114 | P.10 |
| Comb-2050 | 115 | P.10 |
| Comb-2051 | 116 | P.10 |
| Comb-2052 | 117 | P.10 |
| Comb-2053 | 118 | P.10 |
| Comb-2054 | 119 | P.10 |
| Comb-2055 | 120 | P.10 |
| Comb-2056 | 121 | P.10 |
| Comb-2057 | 122 | P.10 |
| Comb-2058 | 123 | P.10 |
| Comb-2059 | 124 | P.10 |
| Comb-2060 | 125 | P.10 |
| Comb-2061 | 126 | P.10 |
| Comb-2062 | 127 | P.10 |
| Comb-2063 | 128 | P.10 |
| Comb-2064 | 129 | P.10 |
| Comb-2065 | 130 | P.10 |
| Comb-2066 | 131 | P.10 |
| Comb-2067 | 132 | P.10 |
| Comb-2068 | 133 | P.10 |
| Comb-2069 | 134 | P.10 |
| Comb-2070 | 135 | P.10 |
| Comb-2071 | 136 | P.10 |
| Comb-2072 | 137 | P.10 |
| Comb-2073 | 138 | P.10 |
| Comb-2074 | 139 | P.10 |
| Comb-2075 | 140 | P.10 |
| Comb-2076 | 141 | P.10 |
| Comb-2077 | 142 | P.10 |
| Comb-2078 | 143 | P.10 |
| Comb-2079 | 144 | P.10 |
| Comb-2080 | 145 | P.10 |
| Comb-2081 | 146 | P.10 |
| Comb-2082 | 147 | P.10 |
| Comb-2083 | 148 | P.10 |
| Comb-2084 | 149 | P.10 |
| Comb-2085 | 150 | P.10 |
| Comb-2086 | 151 | P.10 |
| Comb-2087 | 152 | P.10 |
| Comb-2088 | 153 | P.10 |
| Comb-2089 | 154 | P.10 |
| Comb-2090 | 155 | P.10 |
| Comb-2091 | 156 | P.10 |
| Comb-2092 | 157 | P.10 |
| Comb-2093 | 158 | P.10 |
| Comb-2094 | 159 | P.10 |
| Comb-2095 | 160 | P.10 |
| Comb-2096 | 161 | P.10 |
| Comb-2097 | 162 | P.10 |
| Comb-2098 | 163 | P.10 |
| Comb-2099 | 164 | P.10 |
| Comb-2100 | 165 | P.10 |
| Comb-2101 | 166 | P.10 |
| Comb-2102 | 167 | P.10 |
| Comb-2103 | 168 | P.10 |
| Comb-2104 | 169 | P.10 |
| Comb-2105 | 170 | P.10 |
| Comb-2106 | 171 | P.10 |
| Comb-2107 | 172 | P.10 |
| Comb-2108 | 173 | P.10 |
| Comb-2109 | 174 | P.10 |
| Comb-2110 | 175 | P.10 |
| Comb-2111 | 176 | P.10 |
| Comb-2112 | 177 | P.10 |
| Comb-2113 | 178 | P.10 |
| Comb-2114 | 179 | P.10 |
| Comb-2115 | 180 | P.10 |
| Comb-2116 | 181 | P.10 |
| Comb-2117 | 182 | P.10 |
| Comb-2118 | 183 | P.10 |
| Comb-2119 | 184 | P.10 |
| Comb-2120 | 185 | P.10 |
| Comb-2121 | 186 | P.10 |
| Comb-2122 | 187 | P.10 |
| Comb-2123 | 188 | P.10 |
| Comb-2124 | 189 | P.10 |
| Comb-2125 | 190 | P.10 |
| Comb-2126 | 191 | P.10 |
| Comb-2127 | 192 | P.10 |
| Comb-2128 | 193 | P.10 |
| Comb-2129 | 194 | P.10 |
| Comb-2130 | 195 | P.10 |
| Comb-2131 | 196 | P.10 |
| Comb-2132 | 197 | P.10 |
| Comb-2133 | 198 | P.10 |
| Comb-2134 | 199 | P.10 |
| Comb-2135 | 200 | P.10 |
| Comb-2136 | 201 | P.10 |
| Comb-2137 | 202 | P.10 |
| Comb-2138 | 203 | P.10 |
| Comb-2139 | 204 | P.10 |
| Comb-2140 | 205 | P.10 |
| Comb-2141 | 206 | P.10 |
| Comb-2142 | 207 | P.10 |
| Comb-2143 | 208 | P.10 |
| Comb-2144 | 209 | P.10 |
| Comb-2145 | 210 | P.10 |
| Comb-2146 | 211 | P.10 |
| Comb-2147 | 212 | P.10 |
| Comb-2148 | 213 | P.10 |
| Comb-2149 | 214 | P.10 |
| Comb-2150 | 215 | P.10 |
| Comb-2151 | 1 | P.11 |
| Comb-2152 | 2 | P.11 |
| Comb-2153 | 3 | P.11 |
| Comb-2154 | 4 | P.11 |
| Comb-2155 | 5 | P.11 |
| Comb-2156 | 6 | P.11 |
| Comb-2157 | 7 | P.11 |
| Comb-2158 | 8 | P.11 |
| Comb-2159 | 9 | P.11 |
| Comb-2160 | 10 | P.11 |
| Comb-2161 | 11 | P.11 |
| Comb-2162 | 12 | P.11 |
| Comb-2163 | 13 | P.11 |
| Comb-2164 | 14 | P.11 |
| Comb-2165 | 15 | P.11 |
| Comb-2166 | 16 | P.11 |
| Comb-2167 | 17 | P.11 |
| Comb-2168 | 18 | P.11 |
| Comb-2169 | 19 | P.11 |
| Comb-2170 | 20 | P.11 |
| Comb-2171 | 21 | P.11 |
| Comb-2172 | 22 | P.11 |
| Comb-2173 | 23 | P.11 |
| Comb-2174 | 24 | P.11 |
| Comb-2175 | 25 | P.11 |
| Comb-2176 | 26 | P.11 |
| Comb-2177 | 27 | P.11 |
| Comb-2178 | 28 | P.11 |
| Comb-2179 | 29 | P.11 |
| Comb-2180 | 30 | P.11 |
| Comb-2181 | 31 | P.11 |
| Comb-2182 | 32 | P.11 |
| Comb-2183 | 33 | P.11 |
| Comb-2184 | 34 | P.11 |
| Comb-2185 | 35 | P.11 |
| Comb-2186 | 36 | P.11 |
| Comb-2187 | 37 | P.11 |
| Comb-2188 | 38 | P.11 |
| Comb-2189 | 39 | P.11 |
| Comb-2190 | 40 | P.11 |
| Comb-2191 | 41 | P.11 |
| Comb-2192 | 42 | P.11 |
| Comb-2193 | 43 | P.11 |
| Comb-2194 | 44 | P.11 |
| Comb-2195 | 45 | P.11 |
| Comb-2196 | 46 | P.11 |
| Comb-2197 | 47 | P.11 |
| Comb-2198 | 48 | P.11 |
| Comb-2199 | 49 | P.11 |
| Comb-2200 | 50 | P.11 |
| Comb-2201 | 51 | P.11 |
| Comb-2202 | 52 | P.11 |
| Comb-2203 | 53 | P.11 |
| Comb-2204 | 54 | P.11 |
| Comb-2205 | 55 | P.11 |
| Comb-2206 | 56 | P.11 |
| Comb-2207 | 57 | P.11 |
| Comb-2208 | 58 | P.11 |
| Comb-2209 | 59 | P.11 |
| Comb-2210 | 60 | P.11 |
| Comb-2211 | 61 | P.11 |
| Comb-2212 | 62 | P.11 |
| Comb-2213 | 63 | P.11 |
| Comb-2214 | 64 | P.11 |
| Comb-2215 | 65 | P.11 |
| Comb-2216 | 66 | P.11 |
| Comb-2217 | 67 | P.11 |
| Comb-2218 | 68 | P.11 |
| Comb-2219 | 69 | P.11 |
| Comb-2220 | 70 | P.11 |
| Comb-2221 | 71 | P.11 |
| Comb-2222 | 72 | P.11 |
| Comb-2223 | 73 | P.11 |
| Comb-2224 | 74 | P.11 |
| Comb-2225 | 75 | P.11 |
| Comb-2226 | 76 | P.11 |
| Comb-2227 | 77 | P.11 |
| Comb-2228 | 78 | P.11 |
| Comb-2229 | 79 | P.11 |
| Comb-2230 | 80 | P.11 |
| Comb-2231 | 81 | P.11 |
| Comb-2232 | 82 | P.11 |
| Comb-2233 | 83 | P.11 |
| Comb-2234 | 84 | P.11 |
| Comb-2235 | 85 | P.11 |
| Comb-2236 | 86 | P.11 |
| Comb-2237 | 87 | P.11 |
| Comb-2238 | 88 | P.11 |
| Comb-2239 | 89 | P.11 |
| Comb-2240 | 90 | P.11 |
| Comb-2241 | 91 | P.11 |
| Comb-2242 | 92 | P.11 |
| Comb-2243 | 93 | P.11 |
| Comb-2244 | 94 | P.11 |
| Comb-2245 | 95 | P.11 |
| Comb-2246 | 96 | P.11 |
| Comb-2247 | 97 | P.11 |
| Comb-2248 | 98 | P.11 |
| Comb-2249 | 99 | P.11 |
| Comb-2250 | 100 | P.11 |
| Comb-2251 | 101 | P.11 |
| Comb-2252 | 102 | P.11 |
| Comb-2253 | 103 | P.11 |
| Comb-2254 | 104 | P.11 |
| Comb-2255 | 105 | P.11 |
| Comb-2256 | 106 | P.11 |
| Comb-2257 | 107 | P.11 |
| Comb-2258 | 108 | P.11 |
| Comb-2259 | 109 | P.11 |
| Comb-2260 | 110 | P.11 |
| Comb-2261 | 111 | P.11 |
| Comb-2262 | 112 | P.11 |
| Comb-2263 | 113 | P.11 |
| Comb-2264 | 114 | P.11 |
| Comb-2265 | 115 | P.11 |
| Comb-2266 | 116 | P.11 |
| Comb-2267 | 117 | P.11 |
| Comb-2268 | 118 | P.11 |
| Comb-2269 | 119 | P.11 |
| Comb-2270 | 120 | P.11 |
| Comb-2271 | 121 | P.11 |
| Comb-2272 | 122 | P.11 |
| Comb-2273 | 123 | P.11 |
| Comb-2274 | 124 | P.11 |
| Comb-2275 | 125 | P.11 |
| Comb-2276 | 126 | P.11 |
| Comb-2277 | 127 | P.11 |
| Comb-2278 | 128 | P.11 |
| Comb-2279 | 129 | P.11 |
| Comb-2280 | 130 | P.11 |
| Comb-2281 | 131 | P.11 |
| Comb-2282 | 132 | P.11 |
| Comb-2283 | 133 | P.11 |
| Comb-2284 | 134 | P.11 |
| Comb-2285 | 135 | P.11 |
| Comb-2286 | 136 | P.11 |
| Comb-2287 | 137 | P.11 |
| Comb-2288 | 138 | P.11 |
| Comb-2289 | 139 | P.11 |
| Comb-2290 | 140 | P.11 |
| Comb-2291 | 141 | P.11 |
| Comb-2292 | 142 | P.11 |
| Comb-2293 | 143 | P.11 |
| Comb-2294 | 144 | P.11 |
| Comb-2295 | 145 | P.11 |
| Comb-2296 | 146 | P.11 |
| Comb-2297 | 147 | P.11 |
| Comb-2298 | 148 | P.11 |
| Comb-2299 | 149 | P.11 |
| Comb-2300 | 150 | P.11 |
| Comb-2301 | 151 | P.11 |
| Comb-2302 | 152 | P.11 |
| Comb-2303 | 153 | P.11 |
| Comb-2304 | 154 | P.11 |
| Comb-2305 | 155 | P.11 |
| Comb-2306 | 156 | P.11 |
| Comb-2307 | 157 | P.11 |
| Comb-2308 | 158 | P.11 |
| Comb-2309 | 159 | P.11 |
| Comb-2310 | 160 | P.11 |
| Comb-2311 | 161 | P.11 |
| Comb-2312 | 162 | P.11 |
| Comb-2313 | 163 | P.11 |
| Comb-2314 | 164 | P.11 |
| Comb-2315 | 165 | P.11 |
| Comb-2316 | 166 | P.11 |
| Comb-2317 | 167 | P.11 |
| Comb-2318 | 168 | P.11 |
| Comb-2319 | 169 | P.11 |
| Comb-2320 | 170 | P.11 |
| Comb-2321 | 171 | P.11 |
| Comb-2322 | 172 | P.11 |
| Comb-2323 | 173 | P.11 |
| Comb-2324 | 174 | P.11 |
| Comb-2325 | 175 | P.11 |
| Comb-2326 | 176 | P.11 |
| Comb-2327 | 177 | P.11 |
| Comb-2328 | 178 | P.11 |
| Comb-2329 | 179 | P.11 |
| Comb-2330 | 180 | P.11 |
| Comb-2331 | 181 | P.11 |
| Comb-2332 | 182 | P.11 |
| Comb-2333 | 183 | P.11 |
| Comb-2334 | 184 | P.11 |
| Comb-2335 | 185 | P.11 |
| Comb-2336 | 186 | P.11 |
| Comb-2337 | 187 | P.11 |
| Comb-2338 | 188 | P.11 |
| Comb-2339 | 189 | P.11 |
| Comb-2340 | 190 | P.11 |
| Comb-2341 | 191 | P.11 |
| Comb-2342 | 192 | P.11 |
| Comb-2343 | 193 | P.11 |
| Comb-2344 | 194 | P.11 |
| Comb-2345 | 195 | P.11 |
| Comb-2346 | 196 | P.11 |
| Comb-2347 | 197 | P.11 |
| Comb-2348 | 198 | P.11 |
| Comb-2349 | 199 | P.11 |
| Comb-2350 | 200 | P.11 |
| Comb-2351 | 201 | P.11 |
| Comb-2352 | 202 | P.11 |
| Comb-2353 | 203 | P.11 |
| Comb-2354 | 204 | P.11 |
| Comb-2355 | 205 | P.11 |
| Comb-2356 | 206 | P.11 |
| Comb-2357 | 207 | P.11 |
| Comb-2358 | 208 | P.11 |
| Comb-2359 | 209 | P.11 |
| Comb-2360 | 210 | P.11 |
| Comb-2361 | 211 | P.11 |
| Comb-2362 | 212 | P.11 |
| Comb-2363 | 213 | P.11 |
| Comb-2364 | 214 | P.11 |
| Comb-2365 | 215 | P.11 |
| Comb-2366 | 1 | P.12 |
| Comb-2367 | 2 | P.12 |
| Comb-2368 | 3 | P.12 |
| Comb-2369 | 4 | P.12 |
| Comb-2370 | 5 | P.12 |
| Comb-2371 | 6 | P.12 |
| Comb-2372 | 7 | P.12 |
| Comb-2373 | 8 | P.12 |
| Comb-2374 | 9 | P.12 |
| Comb-2375 | 10 | P.12 |
| Comb-2376 | 11 | P.12 |
| Comb-2377 | 12 | P.12 |
| Comb-2378 | 13 | P.12 |
| Comb-2379 | 14 | P.12 |
| Comb-2380 | 15 | P.12 |
| Comb-2381 | 16 | P.12 |
| Comb-2382 | 17 | P.12 |
| Comb-2383 | 18 | P.12 |
| Comb-2384 | 19 | P.12 |
| Comb-2385 | 20 | P.12 |
| Comb-2386 | 21 | P.12 |
| Comb-2387 | 22 | P.12 |
| Comb-2388 | 23 | P.12 |
| Comb-2389 | 24 | P.12 |
| Comb-2390 | 25 | P.12 |
| Comb-2391 | 26 | P.12 |
| Comb-2392 | 27 | P.12 |
| Comb-2393 | 28 | P.12 |
| Comb-2394 | 29 | P.12 |
| Comb-2395 | 30 | P.12 |
| Comb-2396 | 31 | P.12 |
| Comb-2397 | 32 | P.12 |
| Comb-2398 | 33 | P.12 |
| Comb-2399 | 34 | P.12 |
| Comb-2400 | 35 | P.12 |
| Comb-2401 | 36 | P.12 |
| Comb-2402 | 37 | P.12 |
| Comb-2403 | 38 | P.12 |
| Comb-2404 | 39 | P.12 |
| Comb-2405 | 40 | P.12 |
| Comb-2406 | 41 | P.12 |
| Comb-2407 | 42 | P.12 |
| Comb-2408 | 43 | P.12 |
| Comb-2409 | 44 | P.12 |
| Comb-2410 | 45 | P.12 |
| Comb-2411 | 46 | P.12 |
| Comb-2412 | 47 | P.12 |
| Comb-2413 | 48 | P.12 |
| Comb-2414 | 49 | P.12 |
| Comb-2415 | 50 | P.12 |
| Comb-2416 | 51 | P.12 |
| Comb-2417 | 52 | P.12 |
| Comb-2418 | 53 | P.12 |
| Comb-2419 | 54 | P.12 |
| Comb-2420 | 55 | P.12 |
| Comb-2421 | 56 | P.12 |
| Comb-2422 | 57 | P.12 |
| Comb-2423 | 58 | P.12 |
| Comb-2424 | 59 | P.12 |
| Comb-2425 | 60 | P.12 |
| Comb-2426 | 61 | P.12 |
| Comb-2427 | 62 | P.12 |
| Comb-2428 | 63 | P.12 |
| Comb-2429 | 64 | P.12 |
| Comb-2430 | 65 | P.12 |
| Comb-2431 | 66 | P.12 |
| Comb-2432 | 67 | P.12 |
| Comb-2433 | 68 | P.12 |
| Comb-2434 | 69 | P.12 |
| Comb-2435 | 70 | P.12 |
| Comb-2436 | 71 | P.12 |
| Comb-2437 | 72 | P.12 |
| Comb-2438 | 73 | P.12 |
| Comb-2439 | 74 | P.12 |
| Comb-2440 | 75 | P.12 |
| Comb-2441 | 76 | P.12 |
| Comb-2442 | 77 | P.12 |
| Comb-2443 | 78 | P.12 |
| Comb-2444 | 79 | P.12 |
| Comb-2445 | 80 | P.12 |
| Comb-2446 | 81 | P.12 |
| Comb-2447 | 82 | P.12 |
| Comb-2448 | 83 | P.12 |
| Comb-2449 | 84 | P.12 |
| Comb-2450 | 85 | P.12 |
| Comb-2451 | 86 | P.12 |
| Comb-2452 | 87 | P.12 |
| Comb-2453 | 88 | P.12 |
| Comb-2454 | 89 | P.12 |
| Comb-2455 | 90 | P.12 |
| Comb-2456 | 91 | P.12 |
| Comb-2457 | 92 | P.12 |
| Comb-2458 | 93 | P.12 |
| Comb-2459 | 94 | P.12 |
| Comb-2460 | 95 | P.12 |
| Comb-2461 | 96 | P.12 |
| Comb-2462 | 97 | P.12 |
| Comb-2463 | 98 | P.12 |
| Comb-2464 | 99 | P.12 |
| Comb-2465 | 100 | P.12 |
| Comb-2466 | 101 | P.12 |
| Comb-2467 | 102 | P.12 |
| Comb-2468 | 103 | P.12 |
| Comb-2469 | 104 | P.12 |
| Comb-2470 | 105 | P.12 |
| Comb-2471 | 106 | P.12 |
| Comb-2472 | 107 | P.12 |
| Comb-2473 | 108 | P.12 |
| Comb-2474 | 109 | P.12 |
| Comb-2475 | 110 | P.12 |
| Comb-2476 | 111 | P.12 |
| Comb-2477 | 112 | P.12 |
| Comb-2478 | 113 | P.12 |
| Comb-2479 | 114 | P.12 |
| Comb-2480 | 115 | P.12 |
| Comb-2481 | 116 | P.12 |
| Comb-2482 | 117 | P.12 |
| Comb-2483 | 118 | P.12 |
| Comb-2484 | 119 | P.12 |
| Comb-2485 | 120 | P.12 |
| Comb-2486 | 121 | P.12 |
| Comb-2487 | 122 | P.12 |
| Comb-2488 | 123 | P.12 |
| Comb-2489 | 124 | P.12 |
| Comb-2490 | 125 | P.12 |
| Comb-2491 | 126 | P.12 |
| Comb-2492 | 127 | P.12 |
| Comb-2493 | 128 | P.12 |
| Comb-2494 | 129 | P.12 |
| Comb-2495 | 130 | P.12 |
| Comb-2496 | 131 | P.12 |
| Comb-2497 | 132 | P.12 |
| Comb-2498 | 133 | P.12 |
| Comb-2499 | 134 | P.12 |
| Comb-2500 | 135 | P.12 |
| Comb-2501 | 136 | P.12 |
| Comb-2502 | 137 | P.12 |
| Comb-2503 | 138 | P.12 |
| Comb-2504 | 139 | P.12 |
| Comb-2505 | 140 | P.12 |
| Comb-2506 | 141 | P.12 |
| Comb-2507 | 142 | P.12 |
| Comb-2508 | 143 | P.12 |
| Comb-2509 | 144 | P.12 |
| Comb-2510 | 145 | P.12 |
| Comb-2511 | 146 | P.12 |
| Comb-2512 | 147 | P.12 |
| Comb-2513 | 148 | P.12 |
| Comb-2514 | 149 | P.12 |
| Comb-2515 | 150 | P.12 |
| Comb-2516 | 151 | P.12 |
| Comb-2517 | 152 | P.12 |
| Comb-2518 | 153 | P.12 |
| Comb-2519 | 154 | P.12 |
| Comb-2520 | 155 | P.12 |
| Comb-2521 | 156 | P.12 |
| Comb-2522 | 157 | P.12 |
| Comb-2523 | 158 | P.12 |
| Comb-2524 | 159 | P.12 |
| Comb-2525 | 160 | P.12 |
| Comb-2526 | 161 | P.12 |
| Comb-2527 | 162 | P.12 |
| Comb-2528 | 163 | P.12 |
| Comb-2529 | 164 | P.12 |
| Comb-2530 | 165 | P.12 |
| Comb-2531 | 166 | P.12 |
| Comb-2532 | 167 | P.12 |
| Comb-2533 | 168 | P.12 |
| Comb-2534 | 169 | P.12 |
| Comb-2535 | 170 | P.12 |
| Comb-2536 | 171 | P.12 |
| Comb-2537 | 172 | P.12 |
| Comb-2538 | 173 | P.12 |
| Comb-2539 | 174 | P.12 |
| Comb-2540 | 175 | P.12 |
| Comb-2541 | 176 | P.12 |
| Comb-2542 | 177 | P.12 |
| Comb-2543 | 178 | P.12 |
| Comb-2544 | 179 | P.12 |
| Comb-2545 | 180 | P.12 |
| Comb-2546 | 181 | P.12 |
| Comb-2547 | 182 | P.12 |
| Comb-2548 | 183 | P.12 |
| Comb-2549 | 184 | P.12 |
| Comb-2550 | 185 | P.12 |
| Comb-2551 | 186 | P.12 |
| Comb-2552 | 187 | P.12 |
| Comb-2553 | 188 | P.12 |
| Comb-2554 | 189 | P.12 |
| Comb-2555 | 190 | P.12 |
| Comb-2556 | 191 | P.12 |
| Comb-2557 | 192 | P.12 |
| Comb-2558 | 193 | P.12 |
| Comb-2559 | 194 | P.12 |
| Comb-2560 | 195 | P.12 |
| Comb-2561 | 196 | P.12 |
| Comb-2562 | 197 | P.12 |
| Comb-2563 | 198 | P.12 |
| Comb-2564 | 199 | P.12 |
| Comb-2565 | 200 | P.12 |
| Comb-2566 | 201 | P.12 |
| Comb-2567 | 202 | P.12 |
| Comb-2568 | 203 | P.12 |
| Comb-2569 | 204 | P.12 |
| Comb-2570 | 205 | P.12 |
| Comb-2571 | 206 | P.12 |
| Comb-2572 | 207 | P.12 |
| Comb-2573 | 208 | P.12 |
| Comb-2574 | 209 | P.12 |
| Comb-2575 | 210 | P.12 |
| Comb-2576 | 211 | P.12 |
| Comb-2577 | 212 | P.12 |
| Comb-2578 | 213 | P.12 |
| Comb-2579 | 214 | P.12 |
| Comb-2580 | 215 | P.12 |
| Comb-2581 | 1 | P.13 |
| Comb-2582 | 2 | P.13 |
| Comb-2583 | 3 | P.13 |
| Comb-2584 | 4 | P.13 |
| Comb-2585 | 5 | P.13 |
| Comb-2586 | 6 | P.13 |
| Comb-2587 | 7 | P.13 |
| Comb-2588 | 8 | P.13 |
| Comb-2589 | 9 | P.13 |
| Comb-2590 | 10 | P.13 |
| Comb-2591 | 11 | P.13 |
| Comb-2592 | 12 | P.13 |
| Comb-2593 | 13 | P.13 |
| Comb-2594 | 14 | P.13 |
| Comb-2595 | 15 | P.13 |
| Comb-2596 | 16 | P.13 |
| Comb-2597 | 17 | P.13 |
| Comb-2598 | 18 | P.13 |
| Comb-2599 | 19 | P.13 |
| Comb-2600 | 20 | P.13 |
| Comb-2601 | 21 | P.13 |
| Comb-2602 | 22 | P.13 |
| Comb-2603 | 23 | P.13 |
| Comb-2604 | 24 | P.13 |
| Comb-2605 | 25 | P.13 |
| Comb-2606 | 26 | P.13 |
| Comb-2607 | 27 | P.13 |
| Comb-2608 | 28 | P.13 |
| Comb-2609 | 29 | P.13 |
| Comb-2610 | 30 | P.13 |
| Comb-2611 | 31 | P.13 |
| Comb-2612 | 32 | P.13 |
| Comb-2613 | 33 | P.13 |
| Comb-2614 | 34 | P.13 |
| Comb-2615 | 35 | P.13 |
| Comb-2616 | 36 | P.13 |
| Comb-2617 | 37 | P.13 |
| Comb-2618 | 38 | P.13 |
| Comb-2619 | 39 | P.13 |
| Comb-2620 | 40 | P.13 |
| Comb-2621 | 41 | P.13 |
| Comb-2622 | 42 | P.13 |
| Comb-2623 | 43 | P.13 |
| Comb-2624 | 44 | P.13 |
| Comb-2625 | 45 | P.13 |
| Comb-2626 | 46 | P.13 |
| Comb-2627 | 47 | P.13 |
| Comb-2628 | 48 | P.13 |
| Comb-2629 | 49 | P.13 |
| Comb-2630 | 50 | P.13 |
| Comb-2631 | 51 | P.13 |
| Comb-2632 | 52 | P.13 |
| Comb-2633 | 53 | P.13 |
| Comb-2634 | 54 | P.13 |
| Comb-2635 | 55 | P.13 |
| Comb-2636 | 56 | P.13 |
| Comb-2637 | 57 | P.13 |
| Comb-2638 | 58 | P.13 |
| Comb-2639 | 59 | P.13 |
| Comb-2640 | 60 | P.13 |
| Comb-2641 | 61 | P.13 |
| Comb-2642 | 62 | P.13 |
| Comb-2643 | 63 | P.13 |
| Comb-2644 | 64 | P.13 |
| Comb-2645 | 65 | P.13 |
| Comb-2646 | 66 | P.13 |
| Comb-2647 | 67 | P.13 |
| Comb-2648 | 68 | P.13 |
| Comb-2649 | 69 | P.13 |
| Comb-2650 | 70 | P.13 |
| Comb-2651 | 71 | P.13 |
| Comb-2652 | 72 | P.13 |
| Comb-2653 | 73 | P.13 |
| Comb-2654 | 74 | P.13 |
| Comb-2655 | 75 | P.13 |
| Comb-2656 | 76 | P.13 |
| Comb-2657 | 77 | P.13 |
| Comb-2658 | 78 | P.13 |
| Comb-2659 | 79 | P.13 |
| Comb-2660 | 80 | P.13 |
| Comb-2661 | 81 | P.13 |
| Comb-2662 | 82 | P.13 |
| Comb-2663 | 83 | P.13 |
| Comb-2664 | 84 | P.13 |
| Comb-2665 | 85 | P.13 |
| Comb-2666 | 86 | P.13 |
| Comb-2667 | 87 | P.13 |
| Comb-2668 | 88 | P.13 |
| Comb-2669 | 89 | P.13 |
| Comb-2670 | 90 | P.13 |
| Comb-2671 | 91 | P.13 |
| Comb-2672 | 92 | P.13 |
| Comb-2673 | 93 | P.13 |
| Comb-2674 | 94 | P.13 |
| Comb-2675 | 95 | P.13 |
| Comb-2676 | 96 | P.13 |
| Comb-2677 | 97 | P.13 |
| Comb-2678 | 98 | P.13 |
| Comb-2679 | 99 | P.13 |
| Comb-2680 | 100 | P.13 |
| Comb-2681 | 101 | P.13 |
| Comb-2682 | 102 | P.13 |
| Comb-2683 | 103 | P.13 |
| Comb-2684 | 104 | P.13 |
| Comb-2685 | 105 | P.13 |
| Comb-2686 | 106 | P.13 |
| Comb-2687 | 107 | P.13 |
| Comb-2688 | 108 | P.13 |
| Comb-2689 | 109 | P.13 |
| Comb-2690 | 110 | P.13 |
| Comb-2691 | 111 | P.13 |
| Comb-2692 | 112 | P.13 |
| Comb-2693 | 113 | P.13 |
| Comb-2694 | 114 | P.13 |
| Comb-2695 | 115 | P.13 |
| Comb-2696 | 116 | P.13 |
| Comb-2697 | 117 | P.13 |
| Comb-2698 | 118 | P.13 |
| Comb-2699 | 119 | P.13 |
| Comb-2700 | 120 | P.13 |
| Comb-2701 | 121 | P.13 |
| Comb-2702 | 122 | P.13 |
| Comb-2703 | 123 | P.13 |
| Comb-2704 | 124 | P.13 |
| Comb-2705 | 125 | P.13 |
| Comb-2706 | 126 | P.13 |
| Comb-2707 | 127 | P.13 |
| Comb-2708 | 128 | P.13 |
| Comb-2709 | 129 | P.13 |
| Comb-2710 | 130 | P.13 |
| Comb-2711 | 131 | P.13 |
| Comb-2712 | 132 | P.13 |
| Comb-2713 | 133 | P.13 |
| Comb-2714 | 134 | P.13 |
| Comb-2715 | 135 | P.13 |
| Comb-2716 | 136 | P.13 |
| Comb-2717 | 137 | P.13 |
| Comb-2718 | 138 | P.13 |
| Comb-2719 | 139 | P.13 |
| Comb-2720 | 140 | P.13 |
| Comb-2721 | 141 | P.13 |
| Comb-2722 | 142 | P.13 |
| Comb-2723 | 143 | P.13 |
| Comb-2724 | 144 | P.13 |
| Comb-2725 | 145 | P.13 |
| Comb-2726 | 146 | P.13 |
| Comb-2727 | 147 | P.13 |
| Comb-2728 | 148 | P.13 |
| Comb-2729 | 149 | P.13 |
| Comb-2730 | 150 | P.13 |
| Comb-2731 | 151 | P.13 |
| Comb-2732 | 152 | P.13 |
| Comb-2733 | 153 | P.13 |
| Comb-2734 | 154 | P.13 |
| Comb-2735 | 155 | P.13 |
| Comb-2736 | 156 | P.13 |
| Comb-2737 | 157 | P.13 |
| Comb-2738 | 158 | P.13 |
| Comb-2739 | 159 | P.13 |
| Comb-2740 | 160 | P.13 |
| Comb-2741 | 161 | P.13 |
| Comb-2742 | 162 | P.13 |
| Comb-2743 | 163 | P.13 |
| Comb-2744 | 164 | P.13 |
| Comb-2745 | 165 | P.13 |
| Comb-2746 | 166 | P.13 |
| Comb-2747 | 167 | P.13 |
| Comb-2748 | 168 | P.13 |
| Comb-2749 | 169 | P.13 |
| Comb-2750 | 170 | P.13 |
| Comb-2751 | 171 | P.13 |
| Comb-2752 | 172 | P.13 |
| Comb-2753 | 173 | P.13 |
| Comb-2754 | 174 | P.13 |
| Comb-2755 | 175 | P.13 |
| Comb-2756 | 176 | P.13 |
| Comb-2757 | 177 | P.13 |
| Comb-2758 | 178 | P.13 |
| Comb-2759 | 179 | P.13 |
| Comb-2760 | 180 | P.13 |
| Comb-2761 | 181 | P.13 |
| Comb-2762 | 182 | P.13 |
| Comb-2763 | 183 | P.13 |
| Comb-2764 | 184 | P.13 |
| Comb-2765 | 185 | P.13 |
| Comb-2766 | 186 | P.13 |
| Comb-2767 | 187 | P.13 |
| Comb-2768 | 188 | P.13 |
| Comb-2769 | 189 | P.13 |
| Comb-2770 | 190 | P.13 |
| Comb-2771 | 191 | P.13 |
| Comb-2772 | 192 | P.13 |
| Comb-2773 | 193 | P.13 |
| Comb-2774 | 194 | P.13 |
| Comb-2775 | 195 | P.13 |
| Comb-2776 | 196 | P.13 |
| Comb-2777 | 197 | P.13 |
| Comb-2778 | 198 | P.13 |
| Comb-2779 | 199 | P.13 |
| Comb-2780 | 200 | P.13 |
| Comb-2781 | 201 | P.13 |
| Comb-2782 | 202 | P.13 |
| Comb-2783 | 203 | P.13 |
| Comb-2784 | 204 | P.13 |
| Comb-2785 | 205 | P.13 |
| Comb-2786 | 206 | P.13 |
| Comb-2787 | 207 | P.13 |
| Comb-2788 | 208 | P.13 |
| Comb-2789 | 209 | P.13 |
| Comb-2790 | 210 | P.13 |
| Comb-2791 | 211 | P.13 |
| Comb-2792 | 212 | P.13 |
| Comb-2793 | 213 | P.13 |
| Comb-2794 | 214 | P.13 |
| Comb-2795 | 215 | P.13 |

A composition as defined herein above, in particular a composition comprising as a nitrification inhibitor as defined herein above and a plant growth regulator as defined herein, e.g. a combination of component A and P of Table 2 may be used for the increase of plant health.

The term “plant health” as used herein is intended to mean a condition of the plant which is determined by several aspects alone or in combination with each other. One indicator (indicator 1) for the condition of the plant is the crop yield. “Crop” and “fruit” are to be understood as any plant product which is further utilized after harvesting, e.g. fruits in the proper sense, vegetables, nuts, grains, seeds, wood (e.g. in the case of silviculture plants), flowers (e.g. in the case of gardening plants, ornamentals) etc., that is anything of economic value that is produced by the plant. Another indicator (indicator 2) for the condition of the plant is the plant vigor. The plant vigor becomes manifest in several aspects, too, some of which are visual appearance, e.g. leaf color, fruit color and aspect, amount of dead basal leaves and/or extent of leaf blades, plant weight, plant height, extent of plant verse (lodging), number, strong ness and productivity of tillers, panicles' length, extent of root system, strongness of roots, extent of nodulation, in particular of rhizobial nodulation, point of time of germination, emergence, flowering, grain maturity and/or senescence, protein content, sugar content and the like. Another indicator (indicator 3) for an increase of a plant's health is the reduction of biotic or abiotic stress factors. The three above mentioned indicators for the health condition of a plant may be interdependent and may result from each other. For example, a reduction of biotic or abiotic stress may lead to a better plant vigor, e.g. to better and bigger crops, and thus to an increased yield. Biotic stress, especially over longer terms, can have harmful effects on plants. The term “biotic stress” as used in the context of the present invention refers in particular to stress caused by living organisms. As a result, the quantity and the quality of the stressed plants, their crops and fruits decrease. As far as quality is concerned, reproductive development is usually severely affected with consequences on the crops which are important for fruits or seeds. Growth may be slowed by the stresses; polysaccharide synthesis, both structural and storage, may be reduced or modified: these effects may lead to a decrease in biomass and to changes in the nutritional value of the product. Abiotic stress includes drought, cold, increased UV, increased heat, or other changes in the environment of the plant, that leads to sub-optimal growth conditions. The term “increased yield” of a plant as used herein means that the yield of a product of the respective plant is increased by a measurable amount over the yield of the same product of the plant produced under the same conditions, but without the application of the composition of the invention. According to the present invention, it is preferred that the yield be increased by at least 0.5%, more preferred at least 1%, even more preferred at least 2%, still more preferred at least 4%. An increased yield may, for example, be due to a reduction of nitrification and a corresponding improvement of uptake of nitrogen nutrients. The term “improved plant vigor” as used herein means that certain crop characteristics are increased or improved by a measurable or noticeable amount over the same factor of the plant produced under the same conditions, but without the application of the composition of the present invention. Improved plant vigor can be characterized, among others, by following improved properties of a plant:

(a) improved vitality of the plant,

(b) improved quality of the plant and/or of the plant products, e.g.

(b) enhanced protein content,

(c) improved visual appearance,

(d) delay of senescence,

(e) enhanced root growth and/or more developed root system (e.g. determined by the dry mass of the root),

(f) enhanced nodulation, in particular rhizobial nodulation,

(g) longer panicles,

(h) bigger leaf blade,

(i) less dead basal leaves,

(j) increased chlorophyll content,

(k) prolonged photosynthetically active period,

(l) improved nitrogen-supply within the plant

The improvement of the plant vigor according to the present invention particularly means that the improvement of anyone or several or all of the above mentioned plant characteristics are improved. It further means that if not all of the above characteristics are improved, those which are not improved are not worsened as compared to plants which were not treated according to the invention or are at least not worsened to such an extent that the negative effect exceeds the positive effect of the improved characteristic (i.e. there is always an overall positive effect which preferably results in an improved crop yield). An improved plant vigor may, for example, be due to a reduction of nitrification and, e.g. a regulation of plant growth.

In a further aspect the present invention relates to an agrochemical mixture comprising at least one fertilizer; and at least one nitrification inhibitor as defined as defined herein above; or at least one fertilizer and a composition as mentioned above.

In the terms of the present invention “agrochemical mixture” means a combination of at least two compounds. The term is, however, not restricted to a physical mixture comprising at least two compounds, but refers to any preparation form of at least one compound and at least one further compound, the use of which many be time- and/or locus-related.

The agrochemical mixtures may, for example, be formulated separately but applied in a temporal relationship, i.e. simultaneously or subsequently, the subsequent application having a time interval which allows a combined action of the compounds.

Furthermore, the individual compounds of the agrochemical mixtures according to the invention such as parts of a kit or parts of the binary mixture may be mixed by the user himself in a suitable mixing device. In specific embodiments further auxiliaries may be added, if appropriate.

The term “fertilizers” is to be understood as chemical compounds applied to promote plant and fruit growth. Fertilizers are typically applied either through the soil (for uptake by plant roots), through soil substituents (also for uptake by plant roots), or by foliar feeding (for uptake through leaves). The term also includes mixtures of one or more different types of fertilizers as mentioned below.

The term “fertilizers” can be subdivided into several categories including: a) organic fertilizers (composed of decayed plant/animal matter), b) inorganic fertilizers (composed of chemicals and minerals) and c) urea-containing fertilizers.

Organic fertilizers include manure, e.g. liquid manure, semi-liquid manure, biogas manure, stable manure or straw manure, slurry, worm castings, peat, seaweed, compost, sewage, and guano. Green manure crops are also regularly grown to add nutrients (especially nitrogen) to the soil. Manufactured organic fertilizers include compost, blood meal, bone meal and seaweed extracts. Further examples are enzyme digested proteins, fish meal, and feather meal. The decomposing crop residue from prior years is another source of fertility. In addition, naturally occurring minerals such as mine rock phosphate, sulfate of potash and limestone are also considered inorganic fertilizers.

Inorganic fertilizers are usually manufactured through chemical processes (such as the Haber process), also using naturally occurring deposits, while chemically altering them (e.g. concentrated triple superphosphate). Naturally occurring inorganic fertilizers include Chilean sodium nitrate, mine rock phosphate, and limestone, and raw potash fertilizers.

The inorganic fertilizer may, in a specific embodiment, be a NPK fertilizer. “NPK fertilizers” are inorganic fertilizers formulated in appropriate concentrations and combinations comprising the three main nutrients nitrogen (N), phosphorus (P) and potassium (K) as well as typically S, Mg, Ca, and trace elements.

Urea-containing fertilizer may, in specific embodiments, be urea, formaldehyde urea, urea ammonium nitrate (UAN) solution, urea sulfur, stabilized urea, urea based NPK-fertilizers, or urea ammonium sulfate. Also envisaged is the use of urea as fertilizer. In case urea-containing fertilizers or urea are used or provided, it is particularly preferred that urease inhibitors as defined herein above may be added or additionally be present, or be used at the same time or in connection with the urea-containing fertilizers.

Fertilizers may be provided in any suitable form, e.g. as solid coated or uncoated granules, in liquid or semi-liquid form, as sprayable fertilizer, or via fertigation etc.

Coated fertilizers may be provided with a wide range of materials. Coatings may, for example, be applied to granular or prilled nitrogen (N) fertilizer or to multi-nutrient fertilizers. Typically, urea is used as base material for most coated fertilizers. Alternatively, ammonium or NPK fertilizers are used as base material for coated fertilizers. The present invention, however, also envisages the use of other base materials for coated fertilizers, any one of the fertilizer materials defined herein. In certain embodiments, elemental sulfur may be used as fertilizer coating. The coating may be performed by spraying molten S over urea granules, followed by an application of sealant wax to close fissures in the coating. In a further embodiment, the S layer may be covered with a layer of organic polymers, preferably a thin layer of organic polymers.

Further envisaged coated fertilizers may be provided by reacting resin-based polymers on the surface of the fertilizer granule. A further example of providing coated fertilizers includes the use of low permeability polyethylene polymers in combination with high permeability coatings.

In specific embodiments the composition and/or thickness of the fertilizer coating may be adjusted to control, for example, the nutrient release rate for specific applications. The duration of nutrient release from specific fertilizers may vary, e.g. from several weeks to many months. The presence of nitrification inhibitors in a mixture with coated fertilizers may accordingly be adapted. It is, in particular, envisaged that the nutrient release involves or is accompanied by the release of an nitrification inhibitor according to the present invention.

Coated fertilizers may be provided as controlled release fertilizers (CRFs). In specific embodiments these controlled release fertilizers are fully coated urea or N—P—K fertilizers, which are homogeneous and which typically show a pre-defined longevity of release. In further embodiments, the CRFs may be provided as blended controlled release fertilizer products which may contain coated, uncoated and/or slow release components. In certain embodiments, these coated fertilizers may additionally comprise micronutrients. In specific embodiments these fertilizers may show a pre-defined longevity, e.g. in case of N—P—K fertilizers.

Additionally envisaged examples of CRFs include patterned release fertilizers. These fertilizers typically show a pre-defined release patterns (e.g. hi/standard/lo) and a pre-defined longevity. In exemplary embodiments fully coated N—P—K, Mg and micronutrients may be delivered in a patterned release manner.

Also envisaged are double coating approaches or coated fertilizers based on a programmed release.

In further embodiments the fertilizer mixture may be provided as, or may comprise or contain a slow release fertilizer. The fertilizer may, for example, be released over any suitable period of time, e.g. over a period of 1 to 5 months, preferably up to 3 months. Typical examples of ingredients of slow release fertilizers are IBDU (isobutylidenediurea), e.g. containing about 31-32% nitrogen, of which 90% is water insoluble; or UF, i.e. an urea-formaldehyde product which contains about 38% nitrogen of which about 70% may be provided as water insoluble nitrogen; or CDU (crotonylidene diurea) containing about 32% nitrogen; or MU (methylene urea) containing about 38 to 40% nitrogen, of which 25-60% is typically cold water insoluble nitrogen; or MDU (methylene diurea) containing about 40% nitrogen, of which less than 25% is cold water insoluble nitrogen; or MO (methylol urea) containing about 30% nitrogen, which may typically be used in solutions; or DMTU (diimethylene triurea) containing about 40% nitrogen, of which less than 25% is cold water insoluble nitrogen; or TMTU (tri methylene tetraurea), which may be provided as component of UF products; or TMPU (tri methylene pentaurea), which may also be provided as component of UF products; or UT (urea triazone solution) which typically contains about 28% nitrogen. The fertilizer mixture may also be long-term nitrogen-bearing fertilizer containing a mixture of acetylene diurea and at least one other organic nitrogen-bearing fertilizer selected from methylene urea, isobutylidene diurea, crotonylidene diurea, substituted triazones, triuret or mixtures thereof.

Any of the above mentioned fertilizers or fertilizer forms may suitably be combined. For instance, slow release fertilizers may be provided as coated fertilizers. They may also be combined with other fertilizers or fertilizer types. The same applies to the presence of a nitrification inhibitor according to the present invention, which may be adapted to the form and chemical nature of the fertilizer and accordingly be provided such that its release accompanies the release of the fertilizer, e.g. is released at the same time or with the same frequency. The present invention further envisages fertilizer or fertilizer forms as defined herein above in combination with nitrification inhibitors as defined herein above and further in combination with urease inhibitors as defined herein above. Such combinations may be provided as coated or uncoated forms and/or as slow or fast release forms. Preferred are combinations with slow release fertilizers including a coating. In further embodiments, also different release schemes are envisaged, e.g. a slower or a faster release.

The term “fertigation” as used herein refers to the application of fertilizers, optionally soil amendments, and optionally other water-soluble products together with water through an irrigation system to a plant or to the locus where a plant is growing or is intended to grow, or to a soil substituent as defined herein below. For example, liquid fertilizers or dissolved fertilizers may be provided via fertigation directly to a plant or a locus where a plant is growing or is intended to grow. Likewise, nitrification inhibitors according to the present invention, or in combination with additional nitrification inhibitors, may be provided via fertigation to plants or to a locus where a plant is growing or is intended to grow. Fertilizers and nitrification inhibitors according to the present invention, or in combination with additional nitrification inhibitors, may be provided together, e.g. dissolved in the same charge or load of material (typically water) to be irrigated. In further embodiments, fertilizers and nitrification inhibitors may be provided at different points in time. For example, the fertilizer may be fertigated first, followed by the nitrification inhibitor, or preferably, the nitrification inhibitor may be fertigated first, followed by the fertilizer. The time intervals for these activities follow the herein above outlined time intervals for the application of fertilizers and nitrification inhibitors. Also envisaged is a repeated fertigation of fertilizers and nitrification inhibitors according to the present invention, either together or intermittently, e.g. every 2 hours, 6 hours, 12 hours, 24 hours, 2 days, 3 days, 4 days, 5 days, 6 days or more.

In particularly preferred embodiments, the fertilizer is an ammonium-containing fertilizer.

The agrochemical mixture according to the present invention may comprise one fertilizer as defined herein above and one nitrification inhibitor of formula I as defined herein above. In further embodiments, the agrochemical mixture according to the present invention may comprise at least one or more than one fertilizer as defined herein above, e.g. 2, 3, 4, 5, 6, 6, 7, 8, 9, 10 or more different fertilizers (including inorganic, organic and urea-containing fertilizers) and at least one nitrification inhibitor of formula I as defined herein above, preferably one nitrification inhibitor of formula I selected from Table 1.

In another group of embodiments the agrochemical mixture according to the present invention may comprise at least one or more than one nitrification inhibitor of formula I as defined herein above, preferably more than one nitrification inhibitor of formula I selected from Table 1, e.g. 2, 3, 4, 5, 6, 6, 7, 8, 9, 10 or more different nitrification inhibitors as defined herein above or as provided in Table 1 and at least one fertilizer as defined herein above.

The term “at least one” is to be understood as 1, 2, 3 or more of the respective compound selected from the group consisting of fertilizers as defined herein above (also designated as compound A), and nitrification inhibitors of formula I as defined herein above (also designated as compound B).

In addition to at least one fertilizer and at least one nitrification inhibitor as defined herein above, an agrochemical mixture may comprise further ingredients, compounds, active compounds or compositions or the like. For example, the agrochemical mixture may additionally comprise or composed with or on the basis of a carrier, e.g. an agrochemical carrier, preferably as defined herein. In further embodiments, the agrochemical mixture may further comprise at least one pesticidal compound. For example, the agrochemical mixture may additionally comprise at least one herbicidal compound and/or at least one fungicidal compound and/or at least one insecticidal compound.

In further embodiments, the agrochemical mixture may, in addition to the above indicated ingredients, in particular in addition to the nitrification inhibitor of the compound of formula I and the fertilizer, further comprise alternative or additional nitrification inhibitors such as linoleic acid, alpha-linolenic acid, methyl p-coumarate, methyl ferulate, MHPP, Karanjin, brachialacton, p-benzoquinone sorgoleone, nitrapyrin, dicyandiamide (DCD), 3,4-dimethyl pyrazole phosphate (DMPP), 4-amino-1,2,4-triazole hydrochloride (ATC), 1-amido-2-thiourea (ASU), 2-amino-4-chloro-6-methylpyrimidine (AM), 5-ethoxy-3-trichloromethyl-1,2,4-thiodiazole (terrazole), ammoniumthiosulfate (ATU), 3-methylpyrazol (3-MP), 3,5-dimethylpyrazole (DMP), 1,2,4-triazol and thiourea (TU) and/or sulfathiazole (ST), N-(1H-pyrazolyl-methyl)acetamides such as N-((3(5)-methyl-1H-pyrazole-1-yl)methyl)acetamide, and/or N-(1H-pyrazolyl-methyl)formamides such as N-((3(5)-methyl-1H-pyrazole-1-yl)methyl formamide, N-(4-chloro-3(5)-methyl-pyrazole-1-ylmethyl)-formamide, or N-(3(5),4-dimethyl-pyrazole-1-ylmethyl)-formamide.

Furthermore, the invention relates to a method for reducing nitrification, comprising treating a plant growing on soil and/or the locus where the plant is growing or is intended to grow with at least one nitrification inhibitor as defined herein above, i.e. with an nitrification inhibitor being a compound of formula I, or a derivative thereof, or a composition comprising said nitrification inhibitor.

The term “plant” is to be understood as a plant of economic importance and/or men-grown plant. In certain embodiments, the term may also be understood as plants which have no or no significant economic importance. The plant is preferably selected from agricultural, silvicultural and horticultural (including ornamental) plants. The term also relates to genetically modified plants.

The term “plant” as used herein further includes all parts of a plant such as germinating seeds, emerging seedlings, plant propagules, herbaceous vegetation as well as established woody plants including all belowground portions (such as the roots) and aboveground portions.

Within the context of the method for reducing nitrification it is assumed that the plant is growing on soil. In specific embodiments, the plant may also grow differently, e.g. in synthetic laboratory environments or on soil substituents, or be supplemented with nutrients, water etc. by artificial or technical means. In such scenarios, the invention envisages a treatment of the zone or area where the nutrients, water etc. are provided to the plant. Also envisaged is that the plant grows in green houses or similar indoor facilities.

The term “locus” is to be understood as any type of environment, soil, soil substituent, area or material where the plant is growing or intended to grow. Preferably, the term relates to soil or soil substituent on which a plant is growing.

In one embodiment, the plant to be treated according to the method of the invention is an agricultural plant. “Agricultural plants” are plants of which a part (e.g. seeds) or all is harvested or cultivated on a commercial scale or which serve as an important source of feed, food, fibres (e.g. cotton, linen), combustibles (e.g. wood, bioethanol, biodiesel, biomass) or other chemical compounds. Preferred agricultural plants are for example cereals, e.g. wheat, rye, barley, corn, triticale, oats, sorghum or rice, beet, e.g. sugar beet or fodder beet; fruits, such as pomes, stone fruits or soft fruits, e.g. apples, pears, plums, peaches, almonds, cherries, strawberries, raspberries, blackberries or gooseberries; leguminous plants, such as lentils, peas, alfalfa or soybeans; oil plants, such as rape, oil-seed rape, canola, linseed, mustard, olives, sunflowers, coconut, cocoa beans, castor oil plants, oil palms, ground nuts or soybeans; cucurbits, such as squashes, cucumber or melons; fiber plants, such as cotton, flax, hemp or jute; citrus fruit, such as oranges, lemons, grapefruits or mandarins; vegetables, such as spinach, lettuce, asparagus, cabbages, carrots, onions, tomatoes, potatoes, cucurbits or paprika; lauraceous plants, such as avocados, cinnamon or camphor; energy and raw material plants, such as corn, soybean, rape, canola, sugar cane or oil palm; tobacco; nuts; coffee; tea; bananas; vines (table grapes and grape juice grape vines); hop; turf; natural rubber plants.

In a further embodiment, the plant to be treated according to the method of the invention is a horticultural plant. The term “horticultural plants” are to be understood as plants which are commonly used in horticulture, e.g. the cultivation of ornamentals, vegetables and/or fruits. Examples for ornamentals are turf, geranium, pelargonia, petunia, begonia and fuchsia. Examples for vegetables are potatoes, tomatoes, peppers, cucurbits, cucumbers, melons, watermelons, garlic, onions, carrots, cabbage, beans, peas and lettuce and more preferably from tomatoes, onions, peas and lettuce. Examples for fruits are apples, pears, cherries, strawberry, citrus, peaches, apricots and blueberries.

In a further embodiment, the plant to be treated according to the method of the invention is an ornamental plants. “Ornamental plants” are plants which are commonly used in gardening, e.g. in parks, gardens and on balconies. Examples are turf, geranium, pelargonia, petunia, begonia and fuchsia.

In another embodiment of the present invention, the plant to be treated according to the method of the invention is a silvicultural plants. The term “silvicultural plant” is to be understood as trees, more specifically trees used in reforestation or industrial plantations. Industrial plantations generally serve for the commercial production of forest products, such as wood, pulp, paper, rubber tree, Christmas trees, or young trees for gardening purposes. Examples for silvicultural plants are conifers, like pines, in particular Pinus spec., fir and spruce, eucalyptus, tropical trees like teak, rubber tree, oil palm, willow (Salix), in particular Salix spec., poplar (cottonwood), in particular Populus spec., beech, in particular Fagus spec., birch, oil palm, and oak.

The term “plant propagation material” is to be understood to denote all the generative parts of the plant such as seeds and vegetative plant material such as cuttings and tubers (e.g. potatoes), which can be used for the multiplication of the plant. This includes seeds, grains, roots, fruits, tubers, bulbs, rhizomes, cuttings, spores, offshoots, shoots, sprouts and other parts of plants, including seedlings and young plants, which are to be transplanted after germination or after emergence from soil, meristem tissues, single and multiple plant cells and any other plant tissue from which a complete plant can be obtained.

The term “genetically modified plants” is to be understood as plants, which genetic material has been modified by the use of recombinant DNA techniques in a way that under natural circumstances it cannot readily be obtained by cross breeding, mutations or natural recombination. Typically, one or more genes have been integrated into the genetic material of a genetically modified plant in order to improve certain properties of the plant. Such genetic modifications also include but are not limited to targeted post-translational modification of protein(s), oligo- or polypeptides e.g. by glycosylation or polymer additions such as prenylated, acetylated or farnesylated moieties or PEG moieties.

Plants that have been modified by breeding, mutagenesis or genetic engineering, e.g. have been rendered tolerant to applications of specific classes of herbicides, such as auxin herbicides such as dicamba or 2,4-D; bleacher herbicides such as hydroxylphenylpyruvate dioxygenase (HPPD) inhibitors or phytoene desaturase (PDS) inhibitors; acetolactate synthase (ALS) inhibitors such as sulfonyl ureas or imidazolinones; enolpyruvylshikimate-3-phosphate synthase (EPSPS) inhibitors, such as glyphosate; glutamine synthetase (GS) inhibitors such as glufosinate; protoporphyrinogen-IX oxidase inhibitors; lipid biosynthesis inhibitors such as acetyl CoA carboxylase (ACCase) inhibitors; or oxynil (i.e. bromoxynil or ioxynil) herbicides as a result of conventional methods of breeding or genetic engineering. Furthermore, plants have been made resistant to multiple classes of herbicides through multiple genetic modifications, such as resistance to both glyphosate and glufosinate or to both glyphosate and a herbicide from another class such as ALS inhibitors, HPPD inhibitors, auxin herbicides, or ACCase inhibitors. These herbicide resistance technologies are e.g. described in Pest Managem. Sci. 61, 2005, 246; 61, 2005, 258; 61, 2005, 277; 61, 2005, 269; 61, 2005, 286; 64, 2008, 326; 64, 2008, 332; Weed Sci. 57, 2009, 108; Austral. J. Agricult. Res. 58, 2007, 708; Science 316, 2007, 1185; and references quoted therein. Several cultivated plants have been rendered tolerant to herbicides by conventional methods of breeding (mutagenesis), e.g. Clearfield® summer rape (Canola, BASF SE, Germany) being tolerant to imidazolinones, e.g. imazamox, or ExpressSun® sunflowers (DuPont, USA) being tolerant to sulfonyl ureas, e.g. tribenuron. Genetic engineering methods have been used to render cultivated plants such as soybean, cotton, corn, beets and rape, tolerant to herbicides such as glyphosate and glufosinate, some of which are commercially available under the trade names RoundupReady® (glyphosate-tolerant, Monsanto, U.S.A.), Cultivance® (imidazolinone tolerant, BASF SE, Germany) and LibertyLink® (glufosinate-tolerant, Bayer CropScience, Germany).

Furthermore, plants are also covered that are by the use of recombinant DNA techniques capable to synthesize one or more insecticidal proteins, especially those known from the bacterial genus Bacillus, particularly from Bacillus thuringiensis, such as δ-endotoxins, e.g. CryIA(b), CryIA(c), CryIF, CryIF(a2), CryIIA(b), CryIIIA, CryIIIB(b1) or Cry9c; vegetative insecticidal proteins (VIP), e.g. VIP1, VIP2, VIP3 or VIP3A; insecticidal proteins of bacteria colonizing nematodes, e.g. Photorhabdus spp. or Xenorhabdus spp.; toxins produced by animals, such as scorpion toxins, arachnid toxins, wasp toxins, or other insect-specific neurotoxins; toxins produced by fungi, such Streptomycetes toxins, plant lectins, such as pea or barley lectins; agglutinins; proteinase inhibitors, such as trypsin inhibitors, serine protease inhibitors, patatin, cystatin or papain inhibitors; ribosome-inactivating proteins (RIP), such as ricin, maize-RIP, abrin, luffin, saporin or bryodin; steroid metabolism enzymes, such as 3-hydroxysteroid oxidase, ecdysteroid-IDP-glycosyl-transferase, cholesterol oxidases, ecdysone inhibitors or HMG-CoA-reductase; ion channel blockers, such as blockers of sodium or calcium channels; juvenile hormone esterase; diuretic hormone receptors (helicokinin receptors); stilbene synthase, bibenzyl synthase, chitinases or glucanases. In the context of the present invention these insecticidal proteins or toxins are to be understood expressly also as pre-toxins, hybrid proteins, truncated or otherwise modified proteins. Hybrid proteins are characterized by a new combination of protein domains, (see, e.g. WO 02/015701). Further examples of such toxins or genetically modified plants capable of synthesizing such toxins are disclosed, e.g., in EP-A 374 753, WO 93/007278, WO 95/34656, EP-A 427 529, EP-A 451 878, WO 03/18810 und WO 03/52073.

The methods for producing such genetically modified plants are generally known to the person skilled in the art and are described, e.g. in the publications mentioned above. These insecticidal proteins contained in the genetically modified plants impart to the plants producing these proteins tolerance to harmful pests from all taxonomic groups of arthropods, especially to beetles (Coeloptera), two-winged insects (Diptera), and moths (Lepidoptera) and to nematodes (Nematoda). Genetically modified plants capable to synthesize one or more insecticidal proteins are, e.g., described in the publications mentioned above, and some of which are commercially available such as YieldGard® (corn cultivars producing the Cry1Ab toxin), YieldGard® Plus (corn cultivars producing Cry1Ab and Cry3Bb1 toxins), Starlink® (corn cultivars producing the Cry9c toxin), Herculex® RW (corn cultivars producing Cry34Ab1, Cry35Ab1 and the enzyme phosphinothricin-N-acetyltransferase [PAT]); NuCOTN® 33B (cotton cultivars producing the Cry1Ac toxin), Bollgard® I (cotton cultivars producing the Cry1Ac toxin), Bollgard® II (cotton cultivars producing Cry1Ac and Cry2Ab2 toxins); VIPCOT® (cotton cultivars producing a VIP-toxin); NewLeaf® (potato cultivars producing the Cry3A toxin); Bt-Xtra®, NatureGard®, KnockOut®, BiteGard®, Protecta®, Bt11 (e.g. Agrisure® CB) and Bt176 from Syngenta Seeds SAS, France, (corn cultivars producing the CryIAb toxin and PAT enyzme), MIR604 from Syngenta Seeds SAS, France (corn cultivars producing a modified version of the Cry3A toxin, c.f. WO 03/018810), MON 863 from Monsanto Europe S.A., Belgium (corn cultivars producing the Cry3Bb1 toxin), IPC 531 from Monsanto Europe S.A., Belgium (cotton cultivars producing a modified version of the Cry1Ac toxin) and 1507 from Pioneer Overseas Corporation, Belgium (corn cultivars producing the Cry1F toxin and PAT enzyme).

Furthermore, plants are also covered that are by the use of recombinant DNA techniques capable to synthesize one or more proteins to increase the resistance or tolerance of those plants to bacterial, viral or fungal pathogens. Examples of such proteins are the so-called “pathogenesis-related proteins” (PR proteins, see, e.g. EP-A 392 225), plant disease resistance genes (e.g. potato cultivars, which express resistance genes acting against Phytophthora infestans derived from the Mexican wild potato Solanum bulbocastanum) or T4-lysozym (e.g. potato cultivars capable of synthesizing these proteins with increased resistance against bacteria such as Erwinia amylvora). The methods for producing such genetically modified plants are generally known to the person skilled in the art and are described, e.g. in the publications mentioned above.

Furthermore, plants are also covered that are by the use of recombinant DNA techniques capable to synthesize one or more proteins to increase the productivity (e.g. bio mass production, grain yield, starch content, oil content or protein content), tolerance to drought, salinity or other growth-limiting environmental factors or tolerance to pests and fungal, bacterial or viral pathogens of those plants.

Furthermore, plants are also covered that contain by the use of recombinant DNA techniques a modified amount of substances of content or new substances of content, specifically to improve human or animal nutrition, e.g. oil crops that produce health-promoting long-chain omega-3 fatty acids or unsaturated omega-9 fatty acids (e.g. Nexera® rape, DOW Agro Sciences, Canada).

Furthermore, plants are also covered that contain by the use of recombinant DNA techniques a modified amount of substances of content or new substances of content, specifically to improve raw material production, e.g. potatoes that produce increased amounts of amylopectin (e.g. Amflora® potato, BASF SE, Germany).

The term “soil substituent” as used herein refers to a substrate which is able to allow the growth of a plant and does not comprise usual soil ingredients. This substrate is typically an anorganic substrate which may have the function of an inert medium. It may, in certain embodiments, also comprise organic elements or portions. Soil substituents may, for example, be used in hydroculture or hydroponic approaches, i.e. wherein plants are grown in soilless medium and/or aquatic based environments. Examples of suitable soil substituents, which may be used in the context of the present invention, are perlite, gravel, biochar, mineral wool, coconut husk, phyllosilicates, i.e. sheet silicate minerals, typically formed by parallel sheets of silicate tetrahedra with Si₂O₅ or a 2:5 ratio, or clay aggregates, in particular expanded clay aggregates with a diameter of about 10 to 40 mm. Particularly preferred is the employment of vermiculite, i.e. a phyllosilicate with 2 tetrahedral sheets for every one octahedral sheet present.

The use of soil substituents may, in specific embodiments, be combined with fertigation or irrigation as defined herein.

In specific embodiments, the treatment may be carried out during all suitable growth stages of a plant as defined herein. For example, the treatment may be carried out during the BBCH principle growth stages.

The term “BBCH principal growth stage” refers to the extended BBCH-scale which is a system for a uniform coding of phenologically similar growth stages of all mono- and dicotyledonous plant species in which the entire developmental cycle of the plants is subdivided into clearly recognizable and distinguishable longer-lasting developmental phases. The BBCH-scale uses a decimal code system, which is divided into principal and secondary growth stages. The abbreviation BBCH derives from the Federal Biological Research Centre for Agriculture and Forestry (Germany), the Bundessortenamt (Germany) and the chemical industry.

In one embodiment the invention relates to a method for reducing nitrification comprising treating a plant growing on soil or soil substituents and/or the locus where the plant is growing or is intended to grow with at least one nitrification inhibitor as defined herein above, i.e. with a nitrification inhibitor being a compound of formula I, or a derivative thereof at a growth stage (GS) between GS 00 and GS>BBCH 99 of the pant (e.g. when fertilizing in fall after harvesting apples) and preferably between GS 00 and GS 65 BBCH of the plant.

In one embodiment the invention relates to a method for reducing nitrification comprising treating a plant growing on soil or soil substituents and/or the locus where the plant is growing or is intended to grow with at least one nitrification inhibitor as defined herein above, i.e. with a nitrification inhibitor being a compound of formula I, or a derivative thereof at a growth stage (GS) between GS 00 to GS 45, preferably between GS 00 and GS 40 BBCH, in particular between GS 00 to GS 33 BBCH of the plant.

In a preferred embodiment the invention relates to a method for reducing nitrification comprising treating a plant growing on soil or soil substituents and/or the locus where the plant is growing or is intended to grow with at least one nitrification inhibitor as defined herein above, i.e. with a nitrification inhibitor being a compound of formula I, or a derivative thereof at an early growth stage (GS), in particular a GS 00 to GS 05, or GS 00 to GS 10, or GS 00 to GS 15, or GS 00 to GS 20, or GS 00 to GS 25 or GS 00 to GS 33 BBCH of the plant. In particularly preferred embodiments, the method for reducing nitrification comprises treating a plant growing on soil or soil substituents and/or the locus where the plant is growing or is intended to grow with at least one nitrification inhibitor as defined herein above during growth stages including GS 00.

In a further, specific embodiment of the invention, at least one nitrification inhibitor as defined herein above, i.e. a nitrification inhibitor being a compound of formula I, or a derivative thereof is applied to a plant growing on soil or soil substituents and/or the locus where the plant is growing or is intended to grow at a growth stage between GS 00 and GS 55 BBCH, or of the plant.

In a further embodiment of the invention, at least one nitrification inhibitor as defined herein above, i.e. a nitrification inhibitor being a compound of formula I, or a derivative thereof is applied to a plant growing on soil or soil substituents and/or the locus where the plant is growing or is intended to grow at the growth stage between GS 00 and GS 47 BBCH of the plant.

In one embodiment of the invention, at least one nitrification inhibitor as defined herein above, i.e. a nitrification inhibitor being a compound of formula I, or a derivative thereof is applied to a plant growing on soil or soil substituents and/or the locus where the plant is growing or is intended to grow before and at sowing, before emergence, and until harvest (GS 00 to GS 89 BBCH), or at a growth stage (GS) between GS 00 and GS 65 BBCH of the plant.

In a preferred embodiment the invention relates to a method for reducing nitrification comprising treating a plant growing on soil or soil substituents and/or the locus where the plant is growing with at least one nitrification inhibitor as defined herein above, i.e. with a nitrification inhibitor being a compound of formula I, or a derivative thereof wherein the plant and/or the locus where plant is growing or is intended to grow is additionally provided with at least one fertilizer. The fertilizer may be any suitable fertilizer, preferably a fertilizer as defined herein above. Also envisaged is the application of more than one fertilizer, e.g. 2, 3, 4, 5, 6, 7, 8, 9, 10 fertilizers, or of different fertilizer classes or categories.

In specific embodiments of the invention, at least one nitrification inhibitor as defined herein above, i.e. a nitrification inhibitor being a compound of formula I, or a derivative thereof and at least one fertilizer is applied to a plant growing on soil or soil substituents and/or the locus where the plant is growing or is intended to grow at a growth stage between GS 00 and GS 33 BBCH of the plant.

In specific embodiments of the invention, at least one nitrification inhibitor as defined herein above, i.e. a nitrification inhibitor being a compound of formula I, or a derivative thereof and at least one fertilizer is applied to a plant growing on soil or soil substituents and/or the locus where the plant is growing or is intended to grow at a growth stage between GS 00 and GS 55 BBCH of the plant.

In further specific embodiments of the invention, at least one nitrification inhibitor as defined herein above, i.e. a nitrification inhibitor being a compound of formula I, or a derivative thereof and at least one fertilizer is applied to a plant growing on soil or soil substituents and/or the locus where the plant is growing or is intended to grow at sowing, before emergence, or at a growth stage (GS) between GS 00 and GS>BBCH 99 of the pant (e.g. when fertilizing in fall after harvesting apples) and preferably between GS 00 and 65 BBCH of the plant.

According to a preferred embodiment of the present invention the application of said nitrification inhibitor and of said fertilizer as defined herein above is carried out simultaneously or with a time lag. The term “time lag” as used herein means that either the nitrification inhibitor is applied before the fertilizer to the plant growing on soil or soil substituents and/or the locus where the plant is growing or is intended to grow; or the fertilizer is applied before the nitrification inhibitor to the plant growing on soil or soil substituents and/or the locus where the plant is growing or is intended to grow. Such time lag may be any suitable period of time which still allows to provide a nitrification inhibiting effect in the context of fertilizer usage. For example, the time lag may be a time period of 1 day, 2 days, 3 days, 4 days, 5 days, 6 days, 7 days, 8 days, 9 days, 10 days, 11 days, 12 days, 13 days, 14 days, 3 weeks 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months or more or any time period in between the mentioned time periods. Preferably, the time lag is an interval of 1 day, 2 days, 3 days, 1 week, 2 weeks or 3 weeks. The time lag preferably refers to situations in which the nitrification inhibitor as defined above is provided 1 day, 2 days, 3 days, 4 days, 5 days, 6 days, 7 days, 8 days, 9 days, 10 days, 11 days, 12 days, 13 days, 14 days, 3 weeks 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months or more or any time period in between the mentioned time periods before the application of a fertilizer as defined herein above.

In another specific embodiment of the invention at least one nitrification inhibitor as defined herein above, i.e. a nitrification inhibitor being a compound of formula I, or a derivative thereof is applied between GS 00 to GS 33 BBCH of the plant, or between GS 00 and GS 65 BBCH of the plant, provided that the application of at least one fertilizer as defined herein above is carried out with a time lag of at least 1 day, e.g. a time lag of 1 day, 2 days, 3 days, 4 days, 5 days, 6 days, 7 days, 8 days, 9 days, 10 days, 11 days, 12 days, 13 days, 14 days, 3 weeks 4 weeks, 5 weeks, 6 weeks, 7 weeks, 8 weeks, 9 weeks, 10 weeks, or more or any time period in between the mentioned time periods. It is preferred that the nitrification inhibitors, which is applied between GS 00 to GS 33 BBCH of the plant, is provided 1 day, 2 days, 3 days, 4 days, 5 days, 6 days, 7 days, 8 days, 9 days, 10 days, 11 days, 12 days, 13 days, 14 days, 3 weeks 4 weeks, 5 weeks, 6 weeks, 7 weeks, 8 weeks, 9 weeks, 10 weeks, 11 weeks, or 12 weeks before the application of a fertilizer as defined herein above.

In another specific embodiment of the invention, at least one fertilizer as defined herein above is applied between GS 00 to GS 33 BBCH of the plant or between GS 00 and GS 65 BBCH of the plant, provided that the application of at least one nitrification inhibitor as defined herein above, i.e. of a nitrification inhibitor being a compound of formula I, or a derivative thereof, is carried out with a time lag of at least 1 day, e.g. a time lag of 1 day, 2 days, 3 days, 4 days, 5 days, 6 days, 7 days, 8 days, 9 days, 10 days, 11 days, 12 days, 13 days, 14 days, 3 weeks 4 weeks, 5 weeks, 6 weeks, 7 weeks, 8 weeks, 9 weeks, 10 weeks or more or any time period in between the mentioned time periods.

According to a specific embodiment of the present invention a plant growing on soil or soil substituents and/or the locus where the plant is growing or is intended to grow is treated at least once with a nitrification inhibitor as defined herein above, i.e. with a nitrification inhibitor being a compound of formula I, or a derivative thereof. In a further specific embodiment of the present invention a plant growing on soil or soil substituents and/or the locus where the plant is growing or is intended to grow is treated at least once with a nitrification inhibitor as defined herein above, i.e. with a nitrification inhibitor being a compound of formula I, or a derivative thereof, and at least once with a fertilizer as defined herein above.

The term “at least once” means that the application may be performed one time, or several times, i.e. that a repetition of the treatment with a nitrification inhibitor and/or a fertilizer may be envisaged. Such a repetition may a 2 times, 3 times, 4 times, 5 times, 6 times, 7 times, 8 times, 9 times, 10 times or more frequent repetition of the treatment with a nitrification inhibitor and/or a fertilizer. The repetition of treatment with a nitrification inhibitor and a fertilizer may further be different. For example, while the fertilizer may be applied only once, the nitrification inhibitor may be applied 2 times, 3 times, 4 times etc. Alternatively, while the nitrification inhibitor may be applied only once, the fertilizer may be applied 2 times, 3 times, 4 times etc. Further envisaged are all combination of numerical different numbers of repetitions for the application of a nitrification inhibitor and a fertilizer as defined herein above.

Such a repeated treatment may further be combined with a time lag between the treatment of the nitrification inhibitor and the fertilizer as described above.

The time interval between a first application and second or subsequent application of a nitrification inhibitor and/or a fertilizer may be any suitable interval. This interval may range from a few seconds up to 3 months, e.g. from a few seconds up to 1 month, or from a few seconds up to 2 weeks. In further embodiments, the time interval may range from a few seconds up to 3 days or from 1 second up to 24 hours.

In further specific embodiments, a method for reducing nitrification as described above is carried out by treating a plant growing on soil or soil substituents and/or the locus where the plant is growing or is intended to grow with at least one agrochemical mixture as defined herein above, or with a composition for reducing nitrification as defined herein above.

In another embodiment of the invention, an agrochemical mixture comprising an ammonium or urea-containing fertilizer and at least one nitrification inhibitor as defined herein above is applied before and at sowing, before emergence, and until GS>BBCH 99 of the pant (e.g. when fertilizing in fall after harvesting apples) or until shooting/shoot development (GS 00 to GS 33 BBCH) of the plant. In case the agrochemical mixture is provided as kit of parts or as non-physical mixture, it may be applied with a time lag between the application of the nitrification inhibitor and the fertilizer or between the application of the nitrification inhibitor a secondary or further ingredient, e.g. a pesticidal compound as mentioned herein above.

In a further embodiment plant propagules are preferably treated simultaneously (together or separately) or subsequently.

The term “propagules” or “plant propagules” is to be understood to denote any structure with the capacity to give rise to a new plant, e.g. a seed, a spore, or a part of the vegetative body capable of independent growth if detached from the parent. In a preferred embodiment, the term “propagules” or “plant propagules” denotes for seed.

For a method as described above, or for a use according to the invention, in particular for seed treatment and in furrow application, the application rates of nitrification inhibitors, i.e. of the compound of formula I, are between 0.01 g and 5 kg of active ingredient per hectare, preferably between 1 g and 1 kg of active ingredient per hectare, especially preferred between 50 g and 300 g of active ingredient per hectare depending on different parameters such as the specific active ingredient applied and the plant species treated. In other embodiments of the invention, the compound of formula I is used in an amount of from 0.1 to 1.2 kg per hectare, preferably in an amount of from 0.5 to 1 kg per hectare. In the treatment of seed, amounts of from 0.001 g to 20 g per kg of seed, preferably from 0.01 g to 10 g per kg of seed, more preferably from 0.05 to 2 g per kg of seed of nitrification inhibitors may be generally required.

As a matter of course, if nitrification inhibitors and fertilizers (or other ingredients), or if mixtures thereof are employed, the compounds may be used in an effective and non-phytotoxic amount. This means that they are used in a quantity which allows to obtain the desired effect but which does not give rise to any phytotoxic symptoms on the treated plant or on the plant raised from the treated propagule or treated soil or soil substituents. For the use according to the invention, the application rates of fertilizers may be between 10 kg and 300 kg per hectare, preferably between 50 kg and 250 kg per hectare. In particular, the application rates of fertilizers may be selected such that the amount of applied N is between 10 kg and 1000 kg per hectare, preferably between 50 kg and 700 kg per hectare.

The nitrification inhibitor compounds according to the invention, e.g. compound I as defined herein above, or derivative thereof as defined herein above can be present in different structural or chemical modifications whose biological activity may differ. They are likewise subject matter of the present invention.

The nitrification inhibitor compounds according to the invention, their N-oxides and/or salts etc. may be converted into customary types of compositions, e.g. agrochemical or agricultural compositons such as solutions, emulsions, suspensions, dusts, powders, pastes and granules.

The composition type depends on the particular intended purpose; in each case, it should ensure a fine and uniform distribution of the compound according to the invention. Examples for composition types are suspensions (SC, 00, FS), emulsifiable concentrates (EC), emulsions (EW, EO, ES), microemulsions (ME), pastes, pastilles, wettable powders or dusts (WP, SP, SS, WS, OP, OS) or granules (GR, FG, GG, MG), which can be watersoluble or wettable, as well as gel formulations for the treatment of plant propagation materials such as seeds (GF). Usually the composition types (e.g. SC, 00, FS, EC, WG, SG, WP, SP, SS, WS, GF) are employed diluted. Composition types such as OP, OS, GR, FG, GG and MG are usually used undiluted.

The compositions are prepared in a known manner (see, for example, U.S. Pat. No. 3,060,084, EP 707 445 (for liquid concentrates), Browning: “Agglomeration”, Chemical Engineering, Dec. 4, 1967, 147-48, Perry's Chemical Engineer's Handbook, 4th Ed., McGraw-Hili, New York, 1963, S. 8-57 und ff. WO 91/13546, U.S. Pat. No. 4,172,714, U.S. Pat. No. 4,144,050, U.S. Pat. No. 3,920,442, U.S. Pat. No. 5,180,587, U.S. Pat. No. 5,232,701, U.S. Pat. No. 5,208,030, GB 2,095,558, U.S. Pat. No. 3,299,566, Klingman: Weed Control as a Science (J. Wiley & Sons, New York, 1961), Hance et al.: Weed Control Handbook (8th Ed., Blackwell Scientific, Oxford, 1989) and Mollet, H. and Grubemann, A.: Formulation technology (Wiley VCH Verlag, Weinheim, 2001). Compositions or mixtures may also comprise auxiliaries which are customary, for example, in agrochemical compositions. The auxiliaries used depend on the particular application form and active substance, respectively.

Examples for suitable auxiliaries are solvents, solid carriers, dispersants or emulsifiers (such as further solubilizers, protective colloids, surfactants and adhesion agents), organic and anorganic thickeners, bactericides, anti-freezing agents, anti-foaming agents, if appropriate colorants and tackifiers or binders (e.g. for seed treatment formulations). Suitable solvents are water, organic solvents such as mineral oil fractions of medium to high boiling point, such as kerosene or diesel oil, furthermore coal tar oils and oils of vegetable or animal origin, aliphatic, cyclic and aromatic hydrocarbons, e.g. toluene, xylene, paraffin, tetrahydronaphthalene, alkylated naphthalenes or their derivatives, alcohols such as methanol, ethanol, propanol, butanol and cyclohexanol, glycols, ketones such as cyclohexanone and gamma-butyrolactone, fatty acid dimethylamides, fatty acids and fatty acid esters and strongly polar solvents, e.g. amines such as N-methylpyrrolidone.

Suitable surfactants (adjuvants, wetters, tackifiers, dispersants or emulsifiers) are alkali metal, alkaline earth metal and ammonium salts of aromatic sulfonic acids, such as ligninsoulfonic acid (Borresperse® types, Borregard, Norway) phenolsulfonic acid, naphthalenesulfonic acid (Morwet® types, Akzo Nobel, U.S.A.), dibutylnaphthalene-sulfonic acid (Nekal® types, BASF, GermanY), and fatty acids, alkylsulfonates, alkylarylsulfonates, alkyl sulfates, laurylether sulfates, fatty alcohol sulfates, and sulfated hexa-, hepta- and octadecanolates, sulfated fatty alcohol glycol ethers, furthermore condensates of naphthalene or of naphthalenesulfonic acid with phenol and formaldehyde, polyoxy-ethylene octylphenyl ether, ethoxylated isooctylphenol, octylphenol, nonylphenol, alkylphenyl polyglycol ethers, tributylphenyl polyglycol ether, tristearylphenyl polyglycol ether, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters, lignin-sulfite waste liquors and proteins, denatured proteins, polysaccharides (e.g. methylcellulose), hydrophobically modified starches, polyvinyl alcohols (Mowiol® types, Clariant, Switzerland), polycarboxylates (Sokolan® types, BASF, Germany), polyalkoxylates, polyvinylamines (Lupasol® types, BASF, Germany), polyvinylpyrrolidone and the copolymers thereof. Examples of suitable thickeners (i.e. compounds that impart a modified flowability to compositions, i.e. high viscosity under static conditions and low viscosity during agitation) are polysaccharides and organic and anorganic clays such as Xanthan gum (Kelzan®, CP Kelco, U.S.A.), Rhodopol® 23 (Rhodia, France), Veegum® (R.T. Vanderbilt, U.S.A.) or Attaclay® (Engelhard Corp., NJ, USA).

In specific embodiments, bactericides may be added for preservation and stabilization of the composition. Examples for suitable bactericides are those based on dichlorophene and benzyl alcohol hemi formal (Proxel® from ICI or Acticide® RS from Thor Chemie and Kathon® MK from Rohm & Haas) and isothiazolinone derivatives such as alkylisothiazolinones and benzisothiazolinones (Acticide® MBS from Thor Chemie).

Examples for suitable anti-freezing agents are ethylene glycol, propylene glycol, urea and glycerin. Examples for anti-foaming agents are silicone emulsions (such as e.g. Silikon® SRE, Wacker, Germany or Rhodorsil®, Rhodia, France), long chain alcohols, fatty acids, salts of fatty acids, fluoroorganic compounds and mixtures thereof.

Suitable colorants are pigments of low water solubility and water-soluble dyes, e.g. rhodamin B, C. I. pigment red 112, C. I. solvent red 1, pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108.

Examples for tackifiers or binders are polyvinylpyrrolidons, polyvinylacetates, polyvinyl alcohols and cellulose ethers (Tylose®, Shin-Etsu, Japan).

Powders, materials for spreading and dusts can be prepared by mixing or concomitantly grinding compound of formula I and, if appropriate, further active substances, with at least one solid carrier. Granules, e.g. coated granules, impregnated granules and homogeneous granules, can be prepared by binding the active substances to solid carriers. Examples of such suitable solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, e.g. ammonium sulfate, ammonium phosphate, ammonium nitrate, ureas, and products of vegetable origin, such as cereal meal, tree bark meal, wood meal and nutshell meal, cellulose powders and other solid carriers.

Examples for composition types are:

i) Water-soluble concentrates (SL, LS) 10 parts by weight of a nitrification inhibitor such as a compound of formula I according to the invention are dissolved in 90 parts by weight of water or in a water-soluble solvent. As an alternative, wetting agents or other auxiliaries are added. The active substance dissolves upon dilution with water. In this way, a composition having a content of 10% by weight of active substance is obtained.

ii) Dispersible concentrates (DC) 20 parts by weight of a nitrification inhibitor such as a compound of formula I according to the invention are dissolved in 70 parts by weight of cyclohexanone with addition of 10 parts by weight of a dispersant, e.g. polyvinylpyrrolidone. Dilution with water gives a dispersion. The active substance content is 20% by weight.

iii) Emulsifiable concentrates (EC) 15 parts by weight of a nitrification inhibitor such as a compound of formula I according to the invention are dissolved in 75 parts by weight of xylene with addition of calcium dodecylbenzenesulfonate and castor oil ethoxylate (in each case 5 parts by weight). Dilution with water gives an emulsion. The composition has an active substance content of 15% by weight.

iv) Emulsions (EW, EO, ES) 25 parts by weight of a nitrification inhibitor such as a compound of formula I according to the invention are dissolved in 35 parts by weight of xylene with addition of calcium dodecylbenzenesulfonate and castor oil ethoxylate (in each case 5 parts by weight). This mixture is introduced into 30 parts by weight of water by means of an emulsifying machine (Ultraturrax) and made into a homogeneous emulsion. Dilution with water gives an emulsion. The composition has an active substance content of 25% by weight.

v) Suspensions (SC, 00, FS) In an agitated ball mill, 20 parts by weight of a nitrification inhibitor such as a compound of formula I according to the invention are comminuted with addition of 10 parts by weight of dispersants and wetting agents and 70 parts by weight of water or an organic solvent to give a fine active substance suspension. Dilution with water gives a stable suspension of the active substance. The active substance content in the composition is 20% by weight.

vi) Water-dispersible granules and water-soluble granules (WG, SG) 50 parts by weight of a nitrification inhibitor such as a compound of formula I according to the invention are ground finely with addition of 50 parts by weight of dispersants and wetting agents and prepared as water-dispersible or water-soluble granules by means of technical appliances (e.g. extrusion, spray tower, fluidized bed). Dilution with water gives a stable dispersion or solution of the active substance. The composition has an active substance content of 50% by weight.

vii) Water-dispersible powders and water-soluble powders (WP, SP, SS, WS) 75 parts by weight of a nitrification inhibitor such as a compound of formula I according to the invention are ground in a rotor-stator mill with addition of 25 parts by weight of dispersants, wetting agents and silica gel. Dilution with water gives a stable dispersion or solution of the active substance. The active substance content of the composition is 75% by weight.

viii) Gel (GF) In an agitated ball mill, 20 parts by weight of a nitrification inhibitor such as a compound of formula I according to the invention are comminuted with addition of 10 parts by weight of dispersants, 1 part by weight of a gelling agent wetters and 70 parts by weight of water or of an organic solvent to give a fine suspension of the active substance. Dilution with water gives a stable suspension of the active substance, whereby a composition with 20% (w/w) of active substance is obtained. 2. Composition types to be applied undiluted

ix) Oustable powders (OP, OS) 5 parts by weight of a nitrification inhibitor such as a compound of formula I according to the invention are ground finely and mixed intimately with 95 parts by weight of finely divided kaolin. This gives a dustable composition having an active substance content of 5% by weight.

x) Granules (GR, FG, GG, MG) 0.5 parts by weight of a nitrification inhibitor such as a compound of formula I according to the invention is ground finely and associated with 99.5 parts by weight of carriers. Current methods are extrusion, spray-drying or the fluidized bed. This gives granules to be applied undiluted having an active substance content of 0.5-10% by weight, preferably an active substance content of 0.5-2% by weight.

xi) ULV solutions (UL) 10 parts by weight of a nitrification inhibitor such as a compound of formula I according to the invention are dissolved in 90 parts by weight of an organic solvent, e.g. xylene. This gives a composition to be applied undiluted having an active substance content of 10% by weight.

The compositions, e.g., agrochemical or agricultural compositions generally comprise between 0.01 and 95%, preferably between 0.1 and 90%, most preferably between 0.5 and 90%, by weight of active substance. The active substances are employed in a purity offrom 90% to 100%, preferably from 95% to 100% (according to NMR spectrum).

Water-soluble concentrates (LS), flowable concentrates (FS), powders for dry treatment (OS), water-dispersible powders for slurry treatment (WS), water-soluble powders (SS), emulsions (ES) emulsifiable concentrates (EC) and gels (GF) are usually employed for the purposes of treatment of plant propagation materials, particularly seeds.

These compositions can be applied to plant propagation materials, particularly seeds, diluted or undiluted.

The compositions in question give, after two-to-tenfold dilution, active substance concentrations of from 0.01 to 60% by weight, preferably from 0.1 to 40% by weight, in the ready-to-use preparations. Application can be carried out before or during sowing.

Methods for applying or treating agrochemical or agricultural compounds or mixtures, or compositions as defined herein, respectively, on to plant propagation material, especially seeds, the plant and/or the locus where the plant is growing or is intended to grow are known in the art, and include dressing, coating, pelleting, dusting, soaking and in-furrow application methods of the propagation material. In a preferred embodiment, the compounds or the compositions thereof, respectively, are applied on to the plant propagation material by a method such that germination is not induced, e.g. by seed dressing, pelleting, coating and dusting.

In a preferred embodiment, a suspension-type (FS) composition may be used. Typically, a FS composition may comprise 1-800 g/l of active substance, 1 200 g/l surfactant, 0 to 200 g/l antifreezing agent, 0 to 400 g/l of binder, 0 to 200 g/l of a pigment and up to 1 liter of a solvent, preferably water.

The active substances can be used as such or in the form of their compositions, e.g. in the form of directly sprayable solutions, powders, suspensions, dispersions, emulsions, oil dispersions, pastes, dustable products, materials for spreading, or granules, by means of spraying, atomizing, dusting, spreading, brushing, immersing or pouring.

The application forms depend entirely on the intended purposes; it is intended to ensure in each case the finest possible distribution of the active substances according to the invention. Aqueous application forms can be prepared from emulsion concentrates, pastes or wettable powders (sprayable powders, oil dispersions) by adding water.

To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active substance concentrations in the ready-to-use preparations can be varied within relatively wide ranges. In general, they are from 0.0001 to 90%, such as from 30 to 80%, e.g. from 35 to 45% or from 65 to 75%, or from 0.0001 to 10%, preferably from 0.001 to 1% by weight of active substance. The active substances may also be used successfully in the ultra-low-volume process (ULV), it being possible to apply compositions comprising over 95% by weight of active substance, or even to apply the active substance without additives.

Various types of oils, wetters, adjuvants, herbicides, bactericides, other fungicides and/or pesticides may be added to the active substances or the compositions comprising them, if appropriate not until immediately prior to use (tank mix). These agents can be admixed with the compositions according to the invention in a weight ratio of 1:100 to 100:1, preferably 1:10 to 10:1.

Adjuvants which can be used are in particular organic modified polysiloxanes such as Break Thru S 240®; alcohol alkoxylates such as Atplus 245®, Atplus MBA 1303®, Plurafac LF 300® and Lutensol ON 30®; EO/PO block polymers, e.g. Pluronic RPE 2035® and Genapol B®; alcohol ethoxylates such as Lutensol XP 80®; and dioctyl sulfosuccinate sodium such as Leophen RA®.

In a further aspect the invention relates to a method for treating a fertilizer or a composition. This treatment includes the application of a nitrification inhibitor which is a compound of formula I as defined herein above to a fertilizer or a a composition. The treatment may accordingly result in the presence of said nitrification inhibitor in a preparation of fertilizers or other compositions. Such treatment may, for example, result in a homogenous distribution of nitrification inhibitors on or in fertilizer preparations. Treatment processes are known to the skilled person and may include, for instance, dressing, coating, pelleting, dusting or soaking. In a specific embodiment, the treatment may be a coating of nitrification inhibitors with fertilizer preparations, or a coating of fertilizers with nitrification inhibitors. The treatment may be based on the use of granulation methods as known to the skilled person, e.g. fluidized bed granulation. The treatment may, in certain embodiments, be performed with a composition comprising the nitrification inhibitor as defined herein above, e.g. comprising besides the inhibitor a carrier or a pesticide or any other suitable additional compound as mentioned above.

In a further specific embodiment, the present invention relates to a method for treating seed or plant propagation material. The term “seed treatment” as used herein refers to or involves steps towards the control of biotic stresses on or in seed and the improvement of shooting and development of plants from seeds. For seed treatment it is evident that a plant suffering from biotic stresses such as fungal or insecticidal attack or which has difficulties obtaining sufficient suitable nitrogen-sources shows reduced germination and emergence leading to poorer plant or crop establishment and vigor, and consequently, to a reduced yield as compared to a plant propagation material which has been subjected to curative or preventive treatment against the relevant pest and which can grow without the damage caused by the biotic stress factor. Methods for treating seed or plant progation material according to the invention thus lead, among other advantages, to an enhanced plant health, a better protection against biotic stresses and an increased plant yield.

Seed treatment methods for applying or treating inventive mixtures and compositions thereof, e.g. compositions or agrochemical compositions as defined herein above, and in particular combinations of nitirification inhibitors as defined herein above and secondary effectors such as pesticides, in particular fungicides, insecticides, nematicides and/or biopesticides and/or biostimulants, to plant propagation material, especially seeds, are known in the art, and include dressing, coating, filmcoating, pelleting and soaking application methods of the propagation material. Such methods are also applicable to the combinations or compositions according to the invention.

In further embodiments, the treatment of seeds is performed with compositions comprising, besides a nitrification inhibitor according to the present invention, e.g. compositions as defined herein above, a fungicide and an insecticide, or a fungicide and a nematicide, or a fungicide and a biopesticide and/or biostimulant, or an instecticide and a nematicide, or an insecticide and a biopesticide and/or biostimulant, or a nematicide and a biopesticide and/or biostimulant, or a combination of a fungicide, insecticide and nematicide, or a combination of a fungicide, insecticide and biopesticide and/or biostimulant, or a combination of an insecticide, nematicide, and biopesticide etc.

In a preferred embodiment, the agricultural composition or combination comprising a nitrification inhibitor according to the present invention, e.g. as defined herein above, is applied or treated on to the plant propagation material by a method such that the germination is not negatively impacted. Accordingly, examples of suitable methods for applying (or treating) a plant propagation material, such as a seed, is seed dressing, seed coating or seed pelleting and alike. It is preferred that the plant propagation material is a seed, seed piece (i.e. stalk) or seed bulb.

Although it is believed that the present method can be applied to a seed in any physiological state, it is preferred that the seed be in a sufficiently durable state that it incurs no damage during the treatment process. Typically, the seed would be a seed that had been harvested from the field; removed from the plant; and separated from any cob, stalk, outer husk, and surrounding pulp or other non-seed plant material. The seed would preferably also be biologically stable to the extent that the treatment would cause no biological damage to the seed. It is believed that the treatment can be applied to the seed at any time between harvest of the seed and sowing of the seed or during the sowing process (seed directed applications). The seed may also be primed either before or after the treatment.

Even distribution of the ingredients in compositions or mixtures as defined herein and adherence thereof to the seeds is desired during propagation material treatment. Treatment could vary from a thin film (dressing) of the formulation containing the combination, for example, a mixture of active ingredient(s), on a plant propagation material, such as a seed, where the original size and/or shape are recognizable to an intermediary state (such as a coating) and then to a thicker film (such as pelleting with many layers of different materials (such as carriers, for example, clays; different formulations, such as of other active ingredients; polymers; and colourants) where the original shape and/or size of the seed is no longer recognizable.

An aspect of the present invention includes application of the composition, e.g. agricultural composition or combination comprising a nitrification inhibitor according to the present invention, e.g. as defined herein above, onto the plant propagation material in a targeted fashion, including positioning the ingredients in the combination onto the entire plant propagation material or on only parts thereof, including on only a single side or a portion of a single side. One of ordinary skill in the art would understand these application methods from the description provided in EP954213B1 and WO06/112700.

The composition, e.g. agricultural composition or combination comprising a nitrification inhibitor according to the present invention, e.g. as defined herein above, can also be used in form of a “pill” or “pellet” or a suitable substrate and placing, or sowing, the treated pill, or substrate, next to a plant propagation material. Such techniques are known in the art, particularly in EP1124414, WO07/67042, and WO07/67044. Application of the composition, e.g. agricultural composition, or combination comprising a nitrification inhibitor according to the present invention, e.g. as defined herein above, onto plant propagation material also includes protecting the plant propagation material treated with the combination of the present invention by placing one or more pesticide- and nitrification inhibitor (NI)-containing particles next to a pesticide- and NI-treated seed, wherein the amount of pesticide is such that the pesticide-treated seed and the pesticide-containing particles together contain an Effective Dose of the pesticide and the pesticide dose contained in the pesticide-treated seed is less than or equal to the Maximal Non-Phytotoxic Dose of the pesticide. Such techniques are known in the art, particularly in WO2005/120226.

Application of the combinations onto the seed also includes controlled release coatings on the seeds, wherein the ingredients of the combinations are incorporated into materials that release the ingredients over time. Examples of controlled release seed treatment technologies are generally known in the art and include polymer films, waxes, or other seed coatings, wherein the ingredients may be incorporated into the controlled release material or applied between layers of materials, or both.

Seed can be treated by applying thereto the compound s present in the inventive mixtures in any desired sequence or simultaneously.

The seed treatment occurs to an unsown seed, and the term “unsown seed” is meant to include seed at any period between the harvest of the seed and the sowing of the seed in the ground for the purpose of germination and growth of the plant.

Treatment to an unsown seed is not meant to include those practices in which the active ingredient is applied to the soil or soil substituents but would include any application practice that would target the seed during the planting process.

Preferably, the treatment occurs before sowing of the seed so that the sown seed has been pre-treated with the combination. In particular, seed coating or seed pelleting are preferred in the treatment of the combinations according to the invention. As a result of the treatment, the ingredients in each combination are adhered on to the seed and therefore available for pest control.

The treated seeds can be stored, handled, sowed and tilled in the same manner as any other active ingredient treated seed.

Solutions for seed treatment (LS), suspoemulsions (SE), flowable concentrates (FS), powders for dry treatment (DS), water-dispersible powders for slurry treatment (WS), water-soluble powders (SS), emulsions (ES), emulsifiable concentrates (EC) and gels (GF) are usually employed for the purposes of treatment of plant propagation materials, particularly seeds. Preferred examples of seed treatment formulation types or soil application for pre-mix compositions are of WS, LS, ES, FS, WG or CS-type.

The compositions in question give, after two-to-tenfold dilution, active components concentrations of from 0.01 to 60% by weight, preferably from 0.1 to 40%, in the ready-to-use preparations. Application can be carried out before or during sowing. Methods for applying or treating compositions or combinations comprising a nitrification inhibitor according to the present invention, e.g. as defined herein above on to plant propagation material, especially seeds include dressing, coating, pelleting, dusting, soaking and in-furrow application methods of the propagation material. Preferably, compositions or combinations comprising a nitrification inhibitor according to the present invention, e.g. as defined herein above are applied on to the plant propagation material by a method such that germination is not induced, e.g. by seed dressing, pelleting, coating and dusting.

Typically, a pre-mix formulation for seed treatment application comprises 0.5 to 99.9 percent, especially 1 to 95 percent, of the desired ingredients, and 99.5 to 0.1 percent, especially 99 to 5 percent, of a solid or liquid adjuvant (including, for example, a solvent such as water), where the auxiliaries can be a surfactant in an amount of 0 to 50 percent, especially 0.5 to 40 percent, based on the pre-mix formulation. Whereas commercial products will preferably be formulated as concentrates (e.g., pre-mix composition (formulation), the end user will normally employ dilute formulations (e.g. tank mix composition).

When employed in plant protection, the total amounts of active components applied are, depending on the kind of effect desired, from 0.001 to 10 kg per ha, preferably from 0.005 to 2 kg per ha, more preferably from 0.05 to 0.9 kg per ha, in particular from 0.1 to 0.75 kg per ha. The application rates may range from about 1×10⁶ to 5×10¹⁵ (or more) CFU/ha. Preferably, the spore concentration is about 1×10⁷ to about 1×10¹¹ CFU/ha. In the case of (entomopathogenic) nematodes as microbial pesticides (e.g. Steinernema feltiae), the application rates preferably range inform about 1×10⁵ to 1×10¹² (or more), more preferably from 1×10⁸ to 1×10¹¹, even more preferably from 5×10⁸ to 1×10¹⁰ individuals (e.g. in the form of eggs, juvenile or any other live stages, preferably in an infetive juvenile stage) per ha.

When employed in plant protection by seed treatment, the amount of compositions or combinations comprising a nitrification inhibitor according to the present invention, e.g. as defined herein above (based on total weight of active components) is in the range from 0.01-10 kg, preferably from 0.1-1000 g, more preferably from 1-100 g per 100 kilogram of plant propagation material (preferably seeds). The application rates with respect to plant propagation material preferably may range from about 1×10⁶ to 1×10¹² (or more) CFU/seed. Preferably, the concentration is about 1×10⁶ to about 1×10¹¹ CFU/seed. Alternatively, the application rates with respect to plant propagation material may range from about 1×10⁷ to 1×10¹⁴ (or more) CFU per 100 kg of seed, preferably from 1×10⁹ to about 1×10¹¹ CFU per 100 kg of seed.

The following example is provided for illustrative purposes. It is thus understood that the example is not to be construed as limiting. The skilled person in the art will clearly be able to envisage further modifications of the principles laid out herein.

### EXAMPLE 1

Soil was sampled fresh from a field (e.g. Limburgerhof), dried and sieved through a 500 μm sieve. Approximately 200 mg of soil were placed into each well of a 48 well plate. Compounds, or DMSO alone, were added at a concentration of 10 ppm, dissolved in 1% DMSO. 6 μmol ammonium sulfate was added per well as well as 4.8 mg NaClO₃.

Subsequently, the samples were incubated at room temperature for up to 72 hrs. After the incubation period 64 mg KCl were added and mixed. 25 μl of the supernatant were placed into a fresh plate and 260 μl of a color reaction solution (from Merck Nr 1.11799.0100) were added.

Measurements were taken with a Tecan plate Reader at 540 nm wavelength.

The results of the measurements (with a dose of 10 ppm) were that all compounds #1 to #215 as shown in Table 1, supra demonstrated an inhibition of ≧10% compared to a control (DMSO only).

The inhibition in % is calculated by the equation [(a−b)/a]×100, wherein a is the nitrification rate in the presence of the control (which is assumed to be 100%), and b is the nitrification rate in the presence of the tested compound.

The phenylpropargylether compounds 68, 71, 72, 73, 74, 77, 79, 81, 113, and 118 of Table 1 above supra demonstrated an inhibition of ≧45%. The results are provided in the following table 1a. In each case, the best rating obtained for a compound is provided.

TABLE 1a

| Compound | Rating |
|---|---|
| 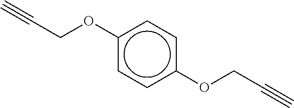 | 66 |
| 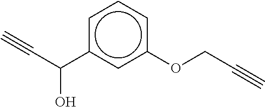 | 61 |
| 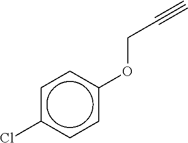 | 59 |
| 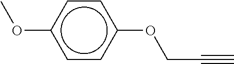 | 58 |
| 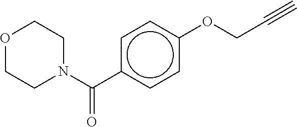 | 57 |
| 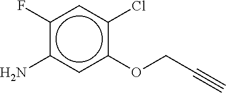 | 62 |
| 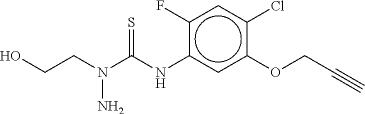 | 56 |
| 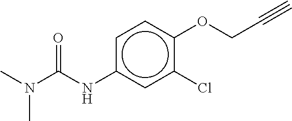 | 55 |
| 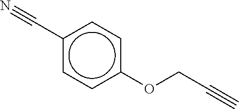 | 53 |
| 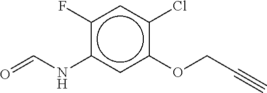 | 48 |

1-16. (canceled)

17: A compound of formula I 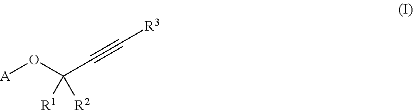
or a stereoisomer, salt, tautomer or N-oxide thereof,
wherein
A is aryl or hetaryl, wherein the aromatic ring may in each case be unsubstituted or may be partially or fully substituted by substituents, which are independently of each other selected from R^{A};
R¹ and R² are independently of each other selected from H and C₁-C₂-alkyl; and
R³ is H, C₁-C₄-haloalkyl, C₁-C₄-hydroxyalkyl, ethynylhydroxymethyl, phenylhydroxymethyl, or aryl, wherein the aromatic ring may be unsubstituted or may be partially or fully substituted by substituents, which are independently of each other selected from R^{B};
and wherein
R^{A} is
(i) halogen, CN, NR^{a}R^{b}, OR^{c}, SR^{c}, C(═Y¹)R^{c}, C(═Y¹)OR^{c}, C(═Y¹)SR^{c}, C(═Y¹)NR^{a}R^{b}, Y²C(═Y¹)R^{c}, Y²C(═Y¹)OR, Y²C(═Y¹)SR^{c}, Y²C(═Y¹)NR^{a}R^{b}, Y³Y²C(═Y¹)R^{c}, NR^{g}N═C(R^{d})(R^{e}), C(═N—OR^{c})R^{g}, C(═N—OR^{c})R^{g}, C(═N—SR^{c})R^{g}, C(═N—NR^{a}R^{b})R^{g}, S(═O)₂R^{f}, NR^{g}S(═O)₂R^{f}, S(═O)₂Y²C(═Y¹)R^{c}, S(═O)₂Y²C(═Y¹)OR^{c}, S(═O)₂Y²C(═Y¹)SR^{c}, S(═O)₂Y²C(═Y¹)NR^{a}R^{b}, NO₂, NON—CN, C₁-C₆-alkyl, C₂-C₆-alkenyl, C₂-C₆-alkynyl, C₁-C₄-haloalkyl, C₁-C₄-cyanoalkyl, C₁-C₄-hydroxyalkyl, C₁-C₄-alkoxy, C₂-C₄-alkynyl-C₁-C₂-hydroxyalkyl, C₂-C₄-alkynyloxy;
(ii) C₁-C₄-alkylene-C(═Y¹)R^{c}, C₂-C₄-alkenylene-C(═Y¹)R^{c}, C₁-C₄-alkylene-C(═Y¹)OR^{c}, C₂-C₄-alkenylene-C(═Y¹)OR^{c}, C₁-C₄-alkylene-C(═Y¹)SR^{c}, C₂-C₄-alkenylene-C(═Y¹)SR^{c}, C₁-C₄-alkylene-C(═Y¹)NR^{a}NR^{b}, C₂-C₄-alkenylene-C(═Y¹)NR^{a}NR^{b}, C₁-C₄-alkylene-Y²—C(═Y¹)R, C₂-C₄-alkenylene-Y²—C(═Y¹)R^{c}, C₁-C₄-alkylene-NR^{a}R^{b}, C₂-C₄-alkenylene-NR^{a}R^{b}, C₁-C₄-alkylene-OR^{c}, C₂-C₄-alkenylene-OR^{c}, C₁-C₄-alkylene-SR^{c}, C₂-C₄-alkenylene-SR^{c}, wherein the C₁-C₄-alkylene or C₂-C₄-alkenylene chain may in each case be unsubstituted or may be partially or fully substituted by OR^{g}, CN, halogen or phenyl;
(iii) aryl, aryl-C₁-C₂-alkyl, hetaryl or hetaryl-C₁-C₂-alkyl, wherein the aromatic ring of the aryl or hetaryl group may be unsubstituted or may be partially or fully substituted by substituents, which are independently of each other selected from R^{h};
(iv) a 3- to 14-membered saturated or unsaturated carbocycle or heterocycle, which may contain 1, 2, or 3 heteroatoms which, independently of each other, are selected from NR^{1b}, O, and S, wherein S may be oxidized and/or wherein the carbocycle or heterocycle may be unsubstituted or may be partially or fully substituted by substituents which, independently of each other, are selected from Rⁱ; and wherein R^{1b} is H, C₁-C₄-alkyl, C₂-C₄-alkenyl, C₃-C₆-cycloalkyl, C₃-C₆-cycloalkylmethyl, or OR^{g}; or
(v) L-B, wherein
L is —CH₂—, —CH═CH—, —C≡C—, —C(═O)— or —CH═, and
B is aryl or hetaryl, wherein the aromatic ring of the ary or hetaryl group may be unsubstituted or may be partially or fully substituted by substituents, which are independently of each other selected from R^{h};
or
a 3- to 14-membered saturated or unsaturated carbocycle or heterocycle, which may contain 1, 2, or 3 heteroatoms which, independently of each other, are selected from NR^{1b}, O, and S, wherein S may be oxidized and/or wherein the carbocycle or heterocycle may be unsubstituted or may be partially or fully substituted by substituents which, independently of each other, are selected from Rⁱ; and wherein R^{1b} is H, C₁-C₄-alkyl, C₂-C₄-alkenyl, C₃-C₆-cycloalkyl, C₃-C₆-cycloalkylmethyl, or OR^{g}; or
(vi) two substituents R^{A} together represent a carbocyclic or heterocyclic ring, which is fused to A and may contain 1, 2, or 3 heteroatoms which, independently of each other, are selected from NR^{1c}, O, and S, wherein S may be oxidized and/or wherein the carbocycle or heterocycle may be unsubstituted or may be partially or fully substituted by substituents which, independently of each other, are selected from Rⁱ; and wherein R^{1c} is H, C₁-C₄-alkyl, C₂-C₄-alkenyl, C₃-C₆-cycloalkyl, C₃-C₆-cycloalkylmethyl, C₃-C₆-heterocyclyl, C₃-C₆-heterocyclylmethyl or OR^{g};
and wherein
R^{B} is NH—C(═O)—(C₁-C₄-alkyl), NH—C(═O)—(C₂-C₄-alkenyl), NH—C(═O)—(C₁-C₂-alkoxy-C₁-C₂-alkyl), NH—C(═O)—(C₃-C₆-cycloalkyl), NH—S(═O)₂—(C₁-C₄-alkyl), or NO₂;
and wherein
Y¹, Y² and Y³ are independently of each other selected from O, S and NR^{1a}, wherein R^{1a} is in each case independently H, C₁-C₄-alkyl, C₂-C₄-alkenyl, C₃-C₆-cycloalkyl, C₃-C₆-cycloalkylmethyl, OR^{g}, SR^{g} or NR^{m}Rⁿ;
R^{a} and R^{b} are independently of each other selected from
(i) H, NR^{j}R^{k}, OR^{l}, SR^{l}, C₁-C₄-alkyl, C₂-C₄-alkenyl, C₂-C₄-alkynyl, C₁-C₄-hydroxyalkyl, C₁-C₄-alkoxy, C(═Y¹)R^{l}, C(═Y¹)OR^{l}, C(═Y¹)SR^{l}, C(═Y¹)NR^{j}R^{k}, C(═Y¹)C(═Y²)R, S(═O)₂R^{f};
(ii) aryl or hetaryl, wherein the aromatic ring of the aryl or hetaryl group may be unsubstituted or may be partially or fully substituted by substituents, which are independently of each other selected from R^{h}; or
R^{a} and R^{b} together with the nitrogen atom to which they are bound form
(iii) a hetaryl group which may be unsubstituted or may be partially or fully substituted by substituents, which are independently of each other selected from R^{h}; or
(iv) a 3- to 10-membered, saturated or unsaturated heterocycle, which may contain 1, 2, or 3 heteroatoms which, independently of each other, are selected from NR^{1b}, O, and S, wherein S may be oxidized and/or wherein the heterocycle may be unsubstituted or may be partially or fully substituted by substituents which, independently of each other, are selected from Rⁱ; and wherein R^{1b} is H, C₁-C₄-alkyl, C₂-C₄-alkenyl, C₃-C₆-cycloalkyl, C₃-C₆-cycloalkylmethyl, or OR^{g};
R^{c} is
(i) H, C₁-C₄-alkyl, C₂-C₄-alkenyl, C₂-C₄-alkynyl, C(═O)OR^{l}, C(═O)SR^{l}, C(═O)NR^{j}R^{k};
(ii) C₁-C₄-alkylene-C(═O)R^{l}, C₁-C₄-alkylene-C(═O)OR^{l}, wherein the C₁-C₄-alkylene chain may in each case be unsubstituted or may be partially or fully substituted by OR^{g}, CN, halogen, or phenyl;
(iii) aryl, aryl-C₁-C₂-alkyl, hetaryl, or hetaryl-C₁-C₂-alkyl, wherein the aromatic ring of the aryl or hetaryl group may be unsubstituted or may be partially or fully substituted by substituents, which are independently of each other selected from R^{h}; or
(iv) a 3- to 10-membered saturated or unsaturated carbocycle or heterocycle, which may contain 1, 2, or 3 heteroatoms which, independently of each other, are selected from NR^{1b}, O, and S, wherein S may be oxidized and/or wherein the carbocycle or heterocycle may be unsubstituted or may be partially or fully substituted by substituents which, independently of each other, are selected from Rⁱ; and wherein R^{1b} is H, C₁-C₄-alkyl, C₂-C₄-alkenyl, C₃-C₆-cycloalkyl, C₃-C₆-cycloalkylmethyl, or OR^{g};
R^{d} and R^{e} are independently selected from C₁-C₄-alkyl, C₁-C₄-haloalkyl, NR^{j}R^{k}, OR^{l}, SR^{l}, CN, C(═Y¹)R^{l}, C(═Y¹)OR^{l}, C(═Y¹)SR^{l}, or C(═Y¹)NR^{j}R^{k};
R^{f} is C₁-C₄-alkyl, C₁-C₄-haloalkyl, NR^{j}R^{k}, OR^{l}, SR^{l}, aryl or hetaryl, wherein the aromatic ring of the aryl or hetaryl group may be unsubstituted or may be partially or fully substituted by substituents, which are independently of each other selected from R^{h};
R^{g} is H or C₁-C₄-alkyl;
R^{h} is halogen, CN, NO₂, NR^{j}R^{k}, OR^{l}, SR^{l}, C₁-C₄-alkyl, C₂-C₄-alkenyl, C₂-C₄-alkynyl, C₁-C₄-haloalkyl, C₁-C₄-alkoxy, C₂-C₄-alkynyloxy, C(═Y¹)R^{l}, C(═Y¹)OR, C(═Y¹)SR^{l}, C(═Y¹)NR^{j}R^{k}, aryl, aryloxy, hetaryl and hetaryloxy;
Rⁱ is
(i) halogen, CN, C₁-C₄-alkyl, C₂-C₄-alkenyl, C₂-C₄-alkynyl, C₁-C₄-haloalkyl, C₂-C₄-haloalkenyl;
(ii) ═NR^{1d}, wherein R^{1d} is H, C₁-C₄-alkyl, C₂-C₄-alkenyl, C₃-C₆-cycloalkyl, C₃-C₆-cycloalkylmethyl, or OR^{g};
(iii) ═O, ═S, NR^{j}R^{k}, OR^{l}, SR^{l}, C(═Y¹)R^{l}, C(═Y¹)OR^{l}, C(═Y¹)SR, C(═Y¹)NR^{j}R^{k};
(iv) aryl, aryl-C₁-C₂-alkyl, hetaryl, or hetaryl-C₁-C₂-alkyl, wherein the aromatic ring of the aryl or hetaryl group may be unsubstituted or may be partially or fully substituted by substituents, which are independently of each other selected from halogen, CN, C₁-C₄-alkyl, C₁-C₄-haloalkyl, C₁-C₄-alkoxy, C₂-C₄-alkynyloxy, OR^{g}, and SR^{g}; or
(v) C₃-C₆-cycloalkyl, or 3- to 6-membered heterocyclyl, wherein the cycloalkyl ring or the heterocyclyl ring may be unsubstituted or may be partially or fully substituted by substituents, which are independently of each other selected from halogen, CN, C₁-C₄-alkyl, OR^{g}, and SR^{g};
R^{j} and R^{k} are independently selected from H, OR^{g}, SR^{g}, C(═Y¹)R^{g}, C(═Y¹)OR^{g}, C(═Y¹)SR^{g}, C(═Y¹)NR^{m}Rⁿ, C₁-C₄-alkyl, C₂-C₄-alkenyl, C₂-C₄-alkynyl, C₁-C₄-haloalkyl, aryl or hetaryl, wherein the aromatic ring of the aryl or hetaryl group may be unsubstituted or may be partially or fully substituted by substituents, which are independently selected from halogen, CN, C₁-C₄-alkyl, C₁-C₄-haloalkyl, C₁-C₄-alkoxy, C₂-C₄-alkynyloxy, OR^{g}, and SR^{g};
R^{l} is H, C₁-C₄-alkyl, C₂-C₄-alkenyl, C₂-C₄-alkynyl, C₁-C₄-haloalkyl, C(═Y¹)R^{g}, C(═Y¹)OR^{g}, C(═Y¹)SR, C(═Y¹)NR^{m}Rⁿ, aryl or hetaryl, wherein the aromatic ring of the aryl or hetaryl group may be unsubstituted or may be partially or fully substituted by substituents, which are independently selected from halogen, CN, C₁-C₄-alkyl, C₁-C₄-haloalkyl, C₁-C₄-alkoxy, C₂-C₄-alkynyloxy, OR^{g}, and SR^{g}; and
R^{m} and Rⁿ are independently selected from H and C₁-C₄-alkyl.

18: The compound of claim 17, wherein in said compound of formula I, A is phenyl or a 6-membered hetaryl, preferably phenyl, wherein the aromatic ring may in each case be unsubstituted or may be partially or fully substituted by substituents, which are independently of each other selected from R^{A}.

19: The compound of claim 17, wherein in said compound of formula I, R¹ and R² both represent hydrogen.

20: The compound of claim 17, wherein in said compound of formula I, R³ is hydrogen, C₁-C₄-haloalkyl or ethinylhydroxymethyl, and preferably R³ is hydrogen.

21: The compound of claim 17, wherein in said compound of formula I, R^{A}, if present, is
(i) halogen, CN, NR^{a}R^{b}, OR^{c}, C(═Y¹)R^{c}, C(═Y¹)OR^{c}, C(═Y¹)SR^{c}, C(═Y¹)NR^{a}R^{b}, Y²C(═Y¹)R, Y²C(═Y¹)NR^{a}R^{b}, NR^{g}N═C(R^{d})(R^{e}), S(═O)₂R^{f}, NO₂, C₁-C₆-alkyl, C₂-C₆- C₁-C₄-haloalkyl, C₁-C₄-alkoxy, C₂-C₄-alkynyl-C₁-C₂-hydroxyalkyl, C₂-C₄-alkynyloxy;
(ii) C₂-C₄-alkenylene-C(═Y¹)R^{c}, C₂-C₄-alkenylene-Y²—C(═Y¹)R^{c}, wherein the C₁-C₄-alkylene or C₂-C₄-alkenylene chain may in each case be unsubstituted or may be partially or fully substituted by CN or halogen;
(iii) aryl, wherein the aromatic ring of the aryl group may be unsubstituted or may be partially or fully substituted by substituents, which are independently of each other selected from R^{h}; or
(iv) a 3- to 14-membered saturated or unsaturated heterocycle, which may contain 1, 2, or 3 heteroatoms which, independently of each other, are selected from NR^{1b}, O, and S, wherein S may be oxidized and/or wherein the heterocycle may be unsubstituted or may be partially or fully substituted by substituents which, independently of each other, are selected from Rⁱ; and wherein R^{1b} is H, C₁-C₄-alkyl, C₂-C₄-alkenyl, C₃-C₆-cycloalkyl, C₃-C₆-cycloalkylmethyl, or OR^{g},
wherein preferably
Y¹, Y² and Y³ are independently of each other selected from O, S and NR^{1a}, wherein R^{1a} is in each case independently H, C₁-C₄-alkyl, OH, or NH₂.
R^{a} and R^{b} are independently of each other selected from
(i) H, NH₂, C₁-C₄-alkyl, C₁-C₄-hydroxyalkyl, C(═O)H, C(═S)H, C(═N—H)H, C(═N—(C₁-C₄)alkyl))H, C(═N—OH)H, C(═N—NH₂)H, or
R^{a} and R^{b} together with the nitrogen atom to which they are bound form
(iv) a 3- to 10-membered, saturated or unsaturated heterocycle, which may contain 1, 2, or 3 heteroatoms which, independently of each other, are selected from NR^{1b}, O, and S, wherein S may be oxidized and/or wherein the heterocycle may be unsubstituted or may be partially or fully substituted by substituents which, independently of each other, are selected from C₁-C₄-alkyl, C₁-C₄-haloalkyl, C₂-C₄-haloalkenyl, and ═O; and wherein R^{1b} is H, C₁-C₄-alkyl, or OH;
R^{c} is
(i) H, C₁-C₄-alkyl; or
(iv) a 3- to 10-membered saturated or unsaturated carbocycle or heterocycle, which may contain 1, 2, or 3 heteroatoms which, independently of each other, are selected from NR^{1b}, O, and S, wherein S may be oxidized and/or wherein the carbocycle or heterocycle may be unsubstituted or may be partially or fully substituted by substituents which, independently of each other, are selected from C₁-C₄-alkyl, C₁-C₄-haloalkyl, C₂-C₄-haloalkenyl, and ═O; and wherein R^{1b} is preferably H, C₁-C₄-alkyl, or OH;
R^{d} and R^{e} are independently selected from NH₂ and C(═O)OH;
R^{f} is C₁-C₄-alkyl;
R^{g} is H;
R^{h} is halogen or C₁-C₄-alkoxy;
and
Rⁱ is
(i) C₁-C₄-alkyl, C₁-C₄-haloalkyl, C₂-C₄-haloalkenyl; or
(iii) ═O.

22: The compound of claim 17, wherein in said compound of formula I,
R¹ and R² both represent hydrogen,
R³ is hydrogen, and
A is phenyl, wherein the aromatic ring is substituted by 1, 2, or 3 substituent(s) R^{A},
wherein the substituent(s) R^{A} are independently of each other selected from halogen, CN, NH₂, C(═O)NR^{a}R^{b}, NHC(═O)NR^{a}R^{b}, NHC(═S)NR^{a}R^{b}, NHC(═O)H, C₁-C₄-alkoxy, C₂-C₄-alkynyl-C₁-C₂-hydroxyalkyl, and C₂-C₄-alkynyloxy,
wherein R^{a} and R^{b} are in each case independently of each other selected from H, C₁-C₂-alkyl, NH₂, C₁-C₂-hydroxyalkyl, or wherein R^{a} and R^{b} may together with the nitrogen atom to which they are bonded form a morpholine ring.

23: A composition for reducing nitrification comprising at least one nitrification inhibitor as defined in claim 17 and at least one carrier.

24: An agrochemical mixture comprising (i) at least one fertilizer; and (ii) at least one nitrification inhibitor as defined in claim 17.

25: A method for reducing nitrification, comprising treating a plant growing on soil or soil substituents and/or the locus or soil or soil substituents where the plant is growing or is intended to grow with at least a compound of claim 17.

26: The method of claim 25, wherein the plant and/or the locus or soil or soil substituents where the plant is growing or is intended to grow is additionally provided with a fertilizer.

27: The method of claim 26, wherein the application of said compound and of said fertilizer is carried out simultaneously or with a time lag, preferably an interval of 1 day, 2 days, 3 days, 1 week, 2 weeks or 3 weeks.

28: The agrochemical mixture of claim 24, wherein said fertilizer is an ammonium-containing inorganic fertilizer such as an NPK fertilizer, anhydrous ammonium, ammonium nitrate, calcium ammonium nitrate, ammonium sulfate nitrate, ammonium sulfate or ammonium phosphate; an organic fertilizer such as liquid manure, semi-liquid manure, biogas manure, stable manure and straw manure, worm castings, compost, seaweed or guano, or an urea-containing fertilizer such as, urea, formaldehyde urea, urea ammonium nitrate (UAN) solution, urea sulphur, stabilized urea, urea based NPK-fertilizers, or urea ammonium sulfate.

29: The method of claim 25, wherein said plant is an agricultural plant such as wheat, barley, oat, rye, soybean, corn, potatoes, oilseed rape, canola, sunflower, cotton, sugar cane, sugar beet, rice, or a vegetable such as spinach, lettuce, asparagus, or cabbages; or sorghum; a silvicultural plant; an ornamental plant; or a horticultural plant, each in its natural or in a genetically modified form.
